(19) United States
(12) Patent Application Publication
(10) Pub. No.: US 2025/0325529 A1
(43) Pub. Date: Oct. 23, 2025

(54) INHIBITORS OF FATTY ACID BINDING PROTEINS (FABPs), METHODS OF USE AND METHODS OF MAKING

(71) Applicant: Celloram, inc., Cleveland, OH (US)

(72) Inventors: Liraz Levi, Pepper Pike, OH (US); Tej Pareek, Beachwood, OH (US); Elizabeth Meyers, Lorain, OH (US); Seunghwan Lim, Solon, OH (US); William J. Greenlee, Teaneck, NJ (US); Seong-Jin Kim, Seocho-Gu (KR)

(73) Assignee: Celloram, Inc., Cleveland, OH (US)

(21) Appl. No.: 19/185,861

(22) Filed: Apr. 22, 2025

Related U.S. Application Data
(60) Provisional application No. 63/637,547, filed on Apr. 23, 2024.
(60) Provisional application No. 63/669,489, filed on Jul. 10, 2024.

(51) Int. Cl. A61K 31/44; A61P 3/10; A61P 35/00; C07D 213/89; C07D 405/12

(52) U.S. Cl. CPC A61K 31/44; A61P 3/10; A61P 35/00; C07D 213/89; C07D 405/12

(57) ABSTRACT

Disclosed herein are FABP inhibitor compounds and their use in pharmaceutical compositions for treating diseases including cancers that highly express any of these FABPs, in particular triple-negative breast cancer (TNBC), hepatocellular carcinoma and other inflammation-induced diseases including cardiovascular disease, obesity or an obesity-related disorders, diabetes, dyslipidemia, impaired glucose tolerance or impaired fasting glucose, vitiligo, psoriasis, viral infection, pain and dementia. Also disclosed herein are methods for preparing the disclosed compounds.

### CROSS REFERENCE TO RELATED APPLICATIONS

This application claims priority of U.S. provisional application No. 63/637,547, filed Apr. 23, 2024, and U.S. provisional application No. 63/669,489, filed Jul. 10, 2024, the disclosure of each of which is hereby incorporated by reference herein.

### FIELD

The present disclosure relates to compounds that inhibit fatty acid binding proteins, such as FABP3, FABP4, FABP5, and/or FABP7, pharmaceutical compositions containing these inhibitor compounds, and uses of these compounds and compositions for treating or preventing cancers that highly express any of these FABPs, in particular triple-negative breast cancer (TNBC), Hepatocellular carcinoma (HCC), autoimmune diseases and disorders, viral infections, and other diseases linked to chronic inflammation including cardiovascular disease, obesity or an obesity-related disorders, diabetes, dyslipidemia, impaired glucose tolerance or impaired fasting glucose, vitiligo, psoriasis, pain and dementia.

### BACKGROUND

Fatty acid binding proteins (FABP) are members of a family of small (12-15 kDa), soluble proteins which contribute to the trafficking of fatty acids within the cytosolic compartments of cells. The proteins are a multigene family, well-conserved, have no catalytic function but transport hydrophobic fatty acids within the aqueous environment of the cytosol to the various destinations enabling fatty acid oxidation, membrane homeostasis or nuclear signaling. In addition, they are involved in signaling processes which are so far poorly understood [1-4]. Structurally, all members of the FABP family share a β-barrel structure that consists of a water-filled cavity and a site that binds specific lipid-ligand unique for each member. FABPs have unique tissue-expression pattern except FABP5 that is ubiquitously expressed in most tissues. Yet, in general, tissues with active lipid metabolism tend to express more than one isoform. FABP3 is mainly expressed in muscle tissues, particularly in the heart and in neurons. FABP4 is highly expressed in adipose tissue, macrophages and endothelial cells. FABP5 is also expressed in macrophages and endothelial cells, as well as in skin, adipocytes, neurons, glia cells, and several other tissues [2, 5, 6]. FABP7 is expressed in the brain, specifically in glia cells and astrocytes. Patient data that became available in recent years indicates FABP5 is highly upregulated in breast tumors, particularly in TNBC tumors. The protein was reported to induce growth and metastasis of TNBC cells, and high levels of the protein are associated with poor survival of TNBC patients. Genetic ablation of FABP5 in the breast cancer mouse model MMTV-NeuT markedly delayed formation of tumors and inhibited their growth rate [7]. And similarly, chemical inhibition of FABP5 suppressed growth of tumors in xenograft models [8, 9]. The data indicates that inhibition of FABP5 is a promising novel approach for treatment of TNBC and perhaps other cancers that highly express this protein.

TNBC is the most aggressive and deadly breast cancer subtype and accounts for 10-20% of all breast cancer cases. Women diagnosed with TNBC are four times more likely to have cancer cells spread or metastasize to other organs within five years than patients with other types of breast cancer. TNBC are a heterogeneous group of breast tumors that are still poorly characterized at the molecular level and lack definitive prognostic markers and selective targets for therapy. This makes the treatment and management of TNBC a significant clinical problem and there is an urgent need for novel targeted therapies for this disease. Current standard of care for TNBC includes neoadjuvant systemic treatment such as anthracyclines, taxanes, and cyclophosphamide. Platinum-based chemotherapy has been proposed but is not yet recommended by available guidelines. Currently, there are no approved targeted therapies for TNBC in the neoadjuvant setting.

FABP5 was shown to be upregulated in HCC tumors and to correlate with poor prognosis of patients. FABP5 promotes the growth of HCC tumors by regulating lipid metabolism to provide the high energy demands of the rapid proliferation of the tumor cells. In addition, the protein modulates the tumor microenvironment by affecting tumor-associated macrophages and their ability to stimulate T-cells and consequently impacting their anti-tumors activity and immunotherapy effectiveness [10, 11].

Genetic deletion of FABP4 and FABP5 in mice improves insulin sensitivity, lowers glucose, and protects against atherosclerosis.4 In a clamp study in ob/ob mice, a specific FABP4 inhibitor (BMS309403) showed a reduction of hepatic glucose production, increased glucose uptake in muscle and adipose tissue, and reduction in hepatic steatosis, but no change in body weight and energy consumption. Additionally, this compound showed a decrease in atherosclerotic plaque formation in ApoE KO mice [2, 3]. In humans, plasma levels of FABP4 are increased in patients with metabolic syndrome and atherosclerosis [12]. In addition, there is growing evidence for involvement of FABP4 in angiogenesis [13] and growth of certain tumors [14]. The global prevalence of obesity is increasing epidemically. Obesity causes an array of health problems, reduces life expectancy, and costs over US $100 billion annually. More than a quarter of the population suffers from an aggregation of co-morbidities, including obesity, atherosclerosis, insulin resistance, dyslipidemias, coagulopathies, hypertension, and a pro-inflammatory state known as the metabolic syndrome. Patients with metabolic syndrome have high risk of athero-sclerosis as well as type 2 diabetes and other health problems. Like obesity, atherosclerosis has very limited therapeutic options.

Atherosclerosis is the leading cause of death in the United States. At the core of this syndrome is the dysregulation of lipid metabolism and aberrant inflammatory responses. Although mechanistic roles for fatty acids have been put forward in the formation of obesity and diabetes by modifying glucose and lipid metabolism as well as inflammatory cascades, little is known about the mechanisms that link fatty acids or other lipid signals to inflammatory responses and the formation of atherosclerotic lesions.

The ability to modulate the immune system offers the prospect of treating wide range of conditions including those caused by inflammation, cancer, and viral infections. Beyond its established role in vaccine development, immunomodulation has therapeutic potential for various conditions including autoimmunity and cancer, as well as inflammatory including metabolic disorders, fibrotic and infectious diseases.

The immune cells play a crucial role in the tumor microenvironment (TME). The tumor-infiltrating immune cells are involved in the regulation of tumor development, progression, and response to treatment. The cellular and molecular profile of the immune TME impacts the disease response to therapy and its outcome by regulating the balance between suppressive versus cytotoxic responses in the vicinity of the tumor [15]. Specific immune cells, such as T cells and natural killer cells, can help suppress tumors' growth and contribute to the elimination of cancer cells. Conversely, the accumulation of immunosuppressive immune cells, such as regulatory T cells and myeloid-derived suppressor cells, can create an environment permissive to tumor growth and progression. The balance between immune-stimulating and immune-suppressive cells in the tumor microenvironment is crucial to the success of immunotherapy and other cancer treatments, Therefore, the ability to manipulate the complex interplay between immune cells and the tumor microenvironment is crucial for the development of more effective cancer therapies.

Targeting immune cells in the tumor microenvironment is a promising strategy for improving the effectiveness of cancer treatments [15-17]. This approach aims to shift the balance of immune cells in the microenvironment from pro-tumor to anti-tumor. One way to do this is by enhancing the activity of immune cells that can recognize and eliminate cancer cells, such as T cells, natural killer cells and the pro-inflammatory M1 macrophages (classically activated macrophages). This can be achieved through the use of immune checkpoint inhibitors, which release the brakes that generally prevent immune cells from attacking the tumor. Another strategy is to reduce the number or activity of immune cells that suppress the immune response, such as regulatory T cells, myeloid-derived suppressor cells or the anti-inflammatory M2 macrophages (alternatively activated macrophages). This can be done through the use of drugs that specifically target these cell types. Combining these and other approaches, such as vaccines or CAR-T cell therapy, may provide a more comprehensive and effective way to target the immune cells in the tumor microenvironment and improve cancer treatment outcomes.

One of the mechanisms by which FABPs regulate immune cells was suggested to be through their role in mediating immune cell metabolism, which is critical for the proper functioning of the immune system By affecting the utilization of fatty acids, FABPs regulate energy production and the signaling pathways involved in the activation and function of immune cells. For example, FABP5 was found to regulate lipid metabolism and function in T-cells in the TME by mediating the uptake and oxidation of long-chain FAs in the cells. Activated T-cells mainly rely on aerobic glycolysis to facilitate their proliferation and anti-tumor function. However, tumor-infiltrating T lymphocytes (TIL) that express high FABP5 levels usually exhibit an exhausted phenotype and impaired anti-tumor activity due to the limited availability of glucose and high levels of long-chain FAs. Inhibition of FABP5 in TILs is therefore expected to activate the anti-tumor activity of the cell by shifting their energy balance [18, 19].

Another Example for regulation of immune cells by FABP5 and FABP4 is regulation of tissue resident T-cells (Trm). Tim cells are a subset of memory T cells which are self-sustaining in non-lymphoid tissues such as the gut, lung, reproductive tract and skin for longer period without circulation and provide the first line of defense against antigens and pathogens through rapid recall responses [20-22]. Trm cells have the ability to regulate local immune homeostasis in tissues and participate in immune responses mediated by pathogens, cancer, and possibly autoantigens during autoimmunity [23]. It has become evident recently that this unique T-cell population contributes to the pathogenesis of autoimmune disorders such as psoriasis, vitiligo, autoimmune hepatitis and rheumatoid arthritis [21]. Due to their specialized function and location within tissues, gene expression signature of Trm cell, as well as their metabolic needs are distinctive from other types of T-cells [22, 24-26]. One of the most distinctive characteristics of Trm cells is they rely on exogenous free fatty acids (FFA), that are internalized from the surrounding environment and metabolized within the cell to produce ATP needed for their maintenance and survival [22]. Recently, it was reported that Trm cells selectively express the fatty acid binding proteins FABP4 and FABP5 that are essential for uptake of FFA into the cells [22]. T-cell-specific deficiency of FABP4/5 impaired uptake of FFA by Trm cells and significantly reduced their longevity and survival in vivo, while having no effect on survival of central memory T (TCM) cells in lymph nodes [22]. Hence, FABP4/5 inhibitors can be used to specifically target Trm in autoimmune diseases.

FABP4 and FABP5 are expressed in macrophages and were shown to regulate their function by facilitating uptake and metabolism of fatty acids and lipid in the cells [27]. FABP4 is highly expressed in Ly6C-MHCII⁻CD36⁺ circulating mnonocyte/macrophages to facilitate oxidative lipid uptake, foam cell formation, angiogenesis, tissue remodeling and pro-tumor functions [27]. FABP5 is highly expressed in Lv6C-MHCII+CD36− macrophages where it was shown to be involved in formation of lipid droplets (LD) in macrophages [28], and in CD11c+ macrophages where it was shown to promote secretion of pro-inflammatory cytokine IL-1b [29, 30], and cause ER stress, exhaustion and ferroptosis [30, 31] and therefor affect immune cell fate and disease progression. This suggests that inhibition of FABP4/5 in macrophages will be beneficial in treating multiple diseases. It is expected to enhance anti-tumor responses in cancer, block formation of foam cells and chronic inflammation in obesity and atherosclerosis, and will promote anti-inflammatory response in cases of inflammatory and autoimmune disease as well as infections. In cases of viral infections, FABP4 was shown to be involved in virus replication and propagation in the case of SARS-Cov2 (COVID-19) and OCT43 (common cold coronavirus) viruses [32]. FABP4 was shown to be recruited to the ER membrane in infected cells and its inhibition was shown to ameliorate viral replication and viral load in cell culture models and to improve disease symptoms in vivo [32]. Overall, the role of FABPs in immune cell regulation highlights the importance of lipid metabolism in the regulation of the immune system and imply that targeting FABPs may be a promising strategy for improving immune cell function and treating immune-related diseases.

Descriptions of FABP inhibitor compounds in the art include the following. U.S. Pat. No. 6,919,323 B2 (Sulsky et al.) describes certain pyridazinone compounds that inhibit the FABP aP2 (FABP4), and the use of these compounds for the treatment of type 2 diabetes and related diseases. U.S. Pat. No. 8,748,470 B2 (Lengyel et al.) describes methods for reducing or inhibiting cancer that include administering to a subject an inhibitor of FABP4 and/or FABP5, where the inhibitor is selected from a list of known compounds, including carbazole butanoic acid, aryl sulfonamide, sulfonylthiophene, 4-hydroxypyrimidine, 2,3-dimethylindole, benzoylbenzene, biphenyl-alkanoic acid, 2-oxazole-alkanoic acid, tetrahydropyrimidone, pyridone, pyrazinone, aryl carboxylic acid, tetrazole, triazolopyrimidinone, indole, or BMS480404. U.S. Pat. No. 8,815,875 B2 (Shipps, Jr. et al.) describes certain heterocyclic compounds that inhibit FABP, and the use of these compounds for the treatment of diseases or disorders including cardiovascular disease, a metabolic disorder, obesity, diabetes, dyslipidemia, and impaired glucose tolerance. U.S. Pat. No. 9,278,918 B2 (Buettelmann, et al.) describes certain urea derivative compounds that inhibit FABP4 and/or FABP5, and the use of these compounds for the treatment of diseases or disorders including type 2 diabetes, atherosclerosis, chronic kidney diseases, and cancer. PCT publication WO2023043803A1 (Levi, et al.) describes certain aniline derivative compounds that inhibit FABP4 and/or FABP5, and the use of these compounds for the treatment of diseases relating to fatty acid metabolism, including cancer.

There remains a need for improved FABP inhibitor compounds, including compounds that inhibit one or more of FABP3, FABP4, FABP5, and FABP7, and uses of these compounds in the treatment of diseases and disorders, including cancer.

### SUMMARY

The present disclosure generally relates to compounds based on a substituted 2-amino-pyridinyl structure that are inhibitors of one or more of FABP3, FABP4, FABP5, and FABP7 (i.e., “FABP3/4/5/7 inhibitors”), and the use of these inhibitors, methods for preparing these inhibitors, and uses of these inhibitors in pharmaceutical compositions for treating diseases relating to fatty acid metabolism. This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides a FABP3/4/5/7 inhibitor compound of structural formula I or pharmaceutically acceptable salt thereof:

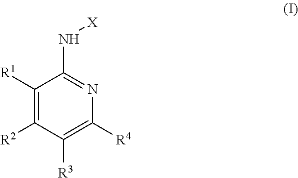

wherein,
   R¹, R², R³, and R⁴ is each independently selected from hydrogen or a substitution for hydrogen;
   X is a moiety of formula:

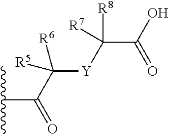

wherein,
   Y is a heteroatom selected from —S—, —O—, —SO—, and —SO₂—, or is —CR⁹R¹⁰—, wherein R⁹ and R¹⁰ are each independently selected from hydrogen, C₁-C₄ linear or branched alkyl, phenyl, and benzyl, or R⁹ and R¹⁰ together form a cyclopropyl, cyclobutyl, or cyclopentyl ring;
   R⁵, R⁶, R⁷, and R⁸ is each independently selected from hydrogen, C₁-C₄ linear or branched alkyl, phenyl, and benzyl, and/or
   R⁵ and R⁶ together or R⁷ and R⁸ together form a cyclopropyl ring, a cyclobutyl ring, a cyclopentyl ring, or an oxetane ring,
   R⁶ and R⁷ together form a 4-, 5-, or 6-membered carbocyclic or heterocyclic ring with Y as a member of the ring, or
   R⁵ and R⁶ together and R⁷ and R⁸ together form a bicyclo[1.1.1] ring.

In at least one embodiment of the FABP3/4/5/7 inhibitor compound of structural formula I, R¹, R², R³, and R⁴ is each independently selected from: hydrogen; halogen; C₁-C₄ linear or branched, saturated or unsaturated alkyl; C₁-C₄ linear or branched, saturated or unsaturated alkoxy; —(CR^{12a}R^{12b})_{q}OR¹³; —(CR^{12a}R^{12b})_{q}C(O)R¹³; —(CR^{12a}R^{12b})_{q}C(O)OR¹³; —O(CR^{12a}R^{12b})_{q}C(O)OR¹³; —(CR^{12a}R^{12b})_{q}N(R¹³)₂; —CHₘXₙ; —(CR^{12a}R^{12b})_{q}CN; and —(CR^{12a}R^{12b})_{q}NO₂; wherein, R¹³ is independently hydrogen, C₁-C₄ linear or branched alkyl, phenyl, or benzyl; and wherein R^{12a} and R^{12b} are each independently hydrogen, methyl (C₁), or ethyl (C₂) and the index q is an integer from 0 to 4, X is halogen, m is 0 to 2, and m+n=3.

In at least one embodiment of the FABP3/4/5/7 inhibitor compound of structural formula I, R¹, R², R³, and R⁴ is each independently selected from: hydrogen; halogen; C₁-C₄ linear, branched, or cyclic, saturated or unsaturated alkyl, phenyl, benzyl, substituted or unsubstituted C₁-C₄ linear, branched, or cyclic, saturated or unsaturated alkoxy, phenoxy, or benzyloxy.

In at least one embodiment of the FABP3/4/5/7 inhibitor compound of structural formula I, R¹, R², R³, and R⁴ is each independently selected from hydrogen, chlorine, fluorine, tri-fluoromethyl, methoxy, difluoro-methoxy, trifluoro-methoxy, ethoxy, propoxy, isopropyloxy, cyclopropyloxy, propargyloxy, butoxy, iso-butoxy, sec-butoxy, tert-butoxy, 2-methoxyethoxy, 2-ethoxy-2-oxoethoxy, (3-methyloxetan-3-yl)oxy, or benzyloxy.

In at least one embodiment of the FABP3/4/5/7 inhibitor compound of structural formula I,
R¹ is selected from chlorine, fluorine, methyl, and tri-fluoromethyl;
R² is hydrogen;
R³ is selected from hydrogen, chlorine, fluorine, methyl, and tri-fluoromethyl; and
R⁴ is each independently selected from methoxy, difluoro-methoxy, trifluoro-methoxy, ethoxy, propoxy, isopropoxy, cyclopropoxy, propargyloxy, butoxy, iso-butoxy, sec-butoxy, tert-butoxy, 2-methoxyethoxy, 2-ethoxy-2-oxoethoxy, (3-methyloxetan-3-yl)oxy, or benzyloxy.

In at least one embodiment of the FABP3/4/5/7 inhibitor compound of structural formula I, the compound of structural formula I has a structural formula selected from Ia, Ib, Ic, Id, Ie, If, Ig, Ih, Ii, Ij, Ik, Il, Im, In, Io, Ip, Iq, Ir, Is, It, Iu, Iv, Iw, Ix, Iy, Iz, Iaa, Ibb, Icc, and Idd (shown in Table 1).

In at least one embodiment of the FABP3/4/5/7 inhibitor compound of structural formula I, Y is selected from —S— or —O— and R⁵, R⁶, R⁷, and R⁸ are each independently hydrogen or C₁-C₄ linear or branched alkyl.

In at least one embodiment of the FABP3/4/5/7 inhibitor compound of structural formula I, R⁵ and R⁶ are hydrogen; and R⁷ and R⁸ are methyl, or R⁷ and R⁸ together form a cyclopropyl ring, cyclobutyl ring, cyclopentyl ring, or oxetane ring.

In at least one embodiment of the FABP3/4/5/7 inhibitor compound of structural formula I, the X moiety is selected from:

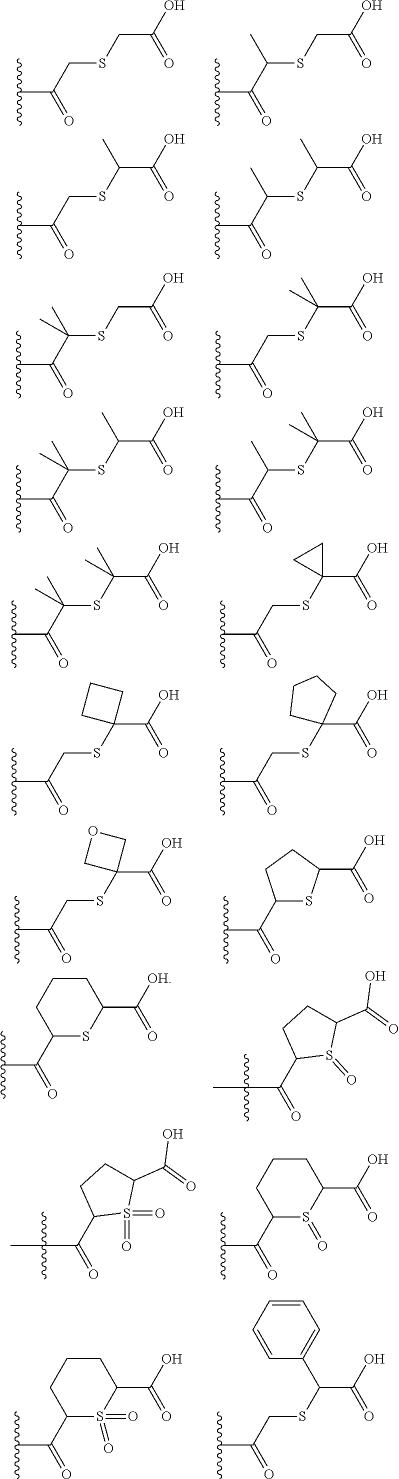

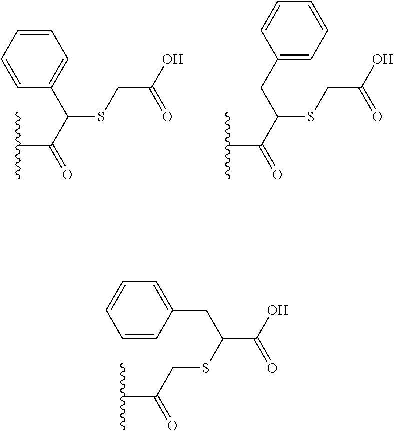 [SMILES: *C(=O)C(SCC(=O)O)c1ccccc1 ; *C(=O)C(Cc1ccccc1)SCC(=O)O ; *C(=O)CSC(Cc1ccccc1)C(=O)O]

In at least one embodiment of the FABP3/4/5/7 inhibitor compound of structural formula I, the X moiety is selected from:

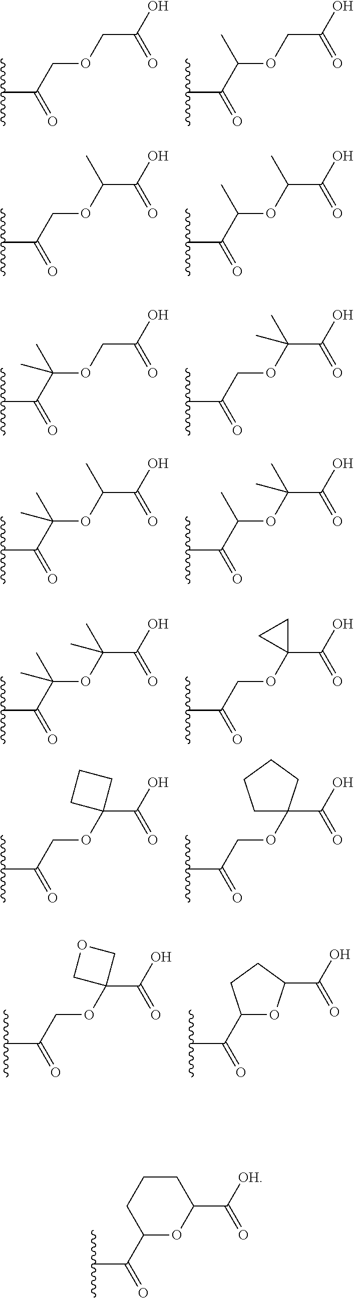

In at least one embodiment of the FABP3/4/5/7 inhibitor compound of structural formula I, the X moiety is selected from:

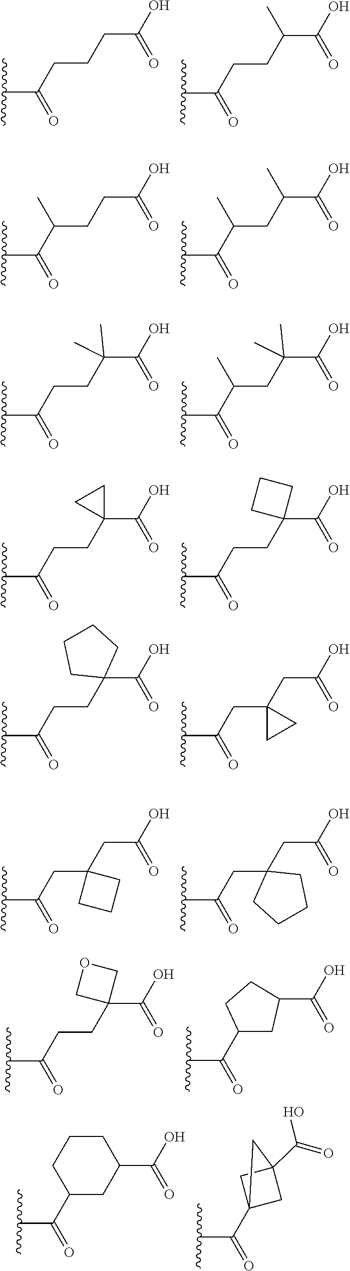

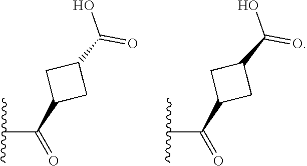

In at least one embodiment, the present disclosure provides a FABP3/4/5/7 inhibitor compound of structural formula II and any pharmaceutically acceptable salt thereof:

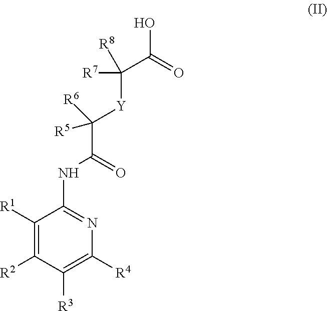

wherein,
   Y is a heteroatom selected from —S—, —O—, —SO—, and —SO₂—, or is —CR⁹R¹⁰—, wherein R⁹ and R¹⁰ are each independently selected from hydrogen, C₁-C₄ linear or branched alkyl, phenyl, and benzyl, or R⁹ and R¹⁰ together form a cyclopropyl, cyclobutyl, or cyclopentyl ring;
   R⁵, R⁶, R⁷, and R⁸ is each independently selected from hydrogen, C₁-C₄ linear or branched alkyl, phenyl, and benzyl, and/or
   R⁵ and R⁶ together or R⁷ and R⁸ together form a cyclopropyl ring, a cyclobutyl ring, a cyclopentyl ring, or an oxetane ring,
   R⁶ and R⁷ together form a 4-, 5-, or 6-membered carbocyclic or heterocyclic ring with Y as a member of the ring, or
   R⁵ and R⁶ together and R⁷ and R⁸ together form a bicyclo[1.1.1] ring.

In at least one embodiment of the FABP3/4/5/7 inhibitor compound of structural formula II, R¹, R², R³, and R⁴ is each independently selected from: hydrogen; halogen; C₁-C₄ linear or branched, saturated or unsaturated alkyl; C₁-C₄ linear or branched, saturated or unsaturated alkoxy; —(CR^{12a}R^{12b})_{q}OR¹³; —(CR^{12a}R^{12b})_{q}C(O)R¹³; —(CR^{12a}R^{12b})_{q}C(O)OR¹³; —O(CR^{12a}R^{12b})_{q}C(O)OR¹³; —(CR^{12a}R^{12b})_{q}N(R¹³)₂; —CHₘXₙ; —(CR^{12a}R^{12b})_{q}CN; and —(CR^{12a}R^{12b})_{q}NO₂; wherein, R¹³ is independently hydrogen, C₁-C₄ linear or branched alkyl, phenyl, or benzyl; and wherein R^{12a} and R^{12b} are each independently hydrogen, methyl (C₁), or ethyl (C₂) and the index q is an integer from 0 to 4, X is halogen, m is 0 to 2, and m+n=3.

In at least one embodiment of the FABP3/4/5/7 inhibitor compound of structural formula II, R¹, R², R³, and R⁴ is each independently selected from: hydrogen; halogen; C₁-C₄ linear, branched, or cyclic, saturated or unsaturated alkyl, phenyl, benzyl, substituted or unsubstituted C₁-C₄ linear, branched, or cyclic, saturated or unsaturated alkoxy, phenoxy, or benzyloxy.

In at least one embodiment of the FABP3/4/5/7 inhibitor compound of structural formula II, R¹, R², R³, and R⁴ is each independently selected from hydrogen, chlorine, fluorine, tri-fluoromethyl, methoxy, difluoro-methoxy, trifluoro-methoxy, ethoxy, propoxy, isopropyloxy, cyclopropyloxy, propargyloxy, butoxy, iso-butoxy, sec-butoxy, tert-butoxy, 2-methoxyethoxy, 2-ethoxy-2-oxoethoxy, (3-methyloxetan-3-yl)oxy, or benzyloxy.

In at least one embodiment of the FABP3/4/5/7 inhibitor compound of structural formula II,
R¹ is selected from chlorine, fluorine, methyl, and tri-fluoromethyl;
R² is hydrogen;
R³ is selected from hydrogen, chlorine, fluorine, methyl, and tri-fluoromethyl; and
R⁴ is each independently selected from methoxy, difluoro-methoxy, trifluoro-methoxy, ethoxy, propoxy, isopropoxy, cyclopropoxy, propargyloxy, butoxy, iso-butoxy, sec-butoxy, tert-butoxy, 2-methoxyethoxy, 2-ethoxy-2-oxoethoxy, (3-methyloxetan-3-yl)oxy, or benzyloxy.

In at least one embodiment of the FABP3/4/5/7 inhibitor compound of structural formula II, the compound of structural formula II has a structural formula selected from IIa, IIb, IIc, IId, IIe, IIf, IIg, IIh, IIi, IIj, IIk, IID, IIm, IIn, IIo, IIp, IIq, IIr, IIs, IIt, IIu, IIv, IIw, IIx, IIy, IIz, IIaa, IIbb, IIcc, and IIdd (shown in Table 2).

In at least one embodiment of the compound of structural formula I and/or the compound of structural formula II, the compound is selected from any of compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60, (shown in Table 3).

In another embodiment, the present disclosure provides a pharmaceutical composition comprising a compound of structural formula I or formula II and one or more adjunct ingredients.

In another embodiment, the present disclosure provides uses of the FABP3/4/5/7 inhibitor compounds of structural formula I or formula II in method of making medicament or pharmaceutical composition for the treatment of a disease or condition affected by any one or more of the FABPs, FABP3, FABP4, FABP5, and FABP7 (i.e., “conditions affected by FABP3/4/5/7”).

In another embodiment, the present disclosure provides a method for treating a subject having a disease or condition affected by FABP3/4/5/7, comprising administering to a subject in need thereof a therapeutically effective amount of compound of structural formula I or formula II, or a pharmaceutical composition comprising a compound of structural formula I or formula II and one or more adjunct ingredients.

In at least one embodiment, the diseases or conditions affected by FABP3/4/5/7 present in a subject which the compounds of structural formula I or formula II can be used to treat can be selected from: atherosclerosis, coronary atherosclerosis, arterial fibrosis, pulmonary hypertension, heart failure, obesity, Type-2 diabetes, conditions affected by lipid metabolism and free fatty acid serum levels, metabolic disorders, fatty liver disease, systemic inflammation, acute inflammation, allergic inflammation, airway inflammation, viral infections (e.g., COVID-19, common cold), dermatology diseases (e.g., vitiligo, psoriasis, atopic dermatitis, atopic march, radiation-induced skin fibrosis, skin inflammation), neurological conditions and diseases (e.g., pain, multiple sclerosis (MS), Parkinson's disease, experimental autoimmune encephalomyelitis (EAE), and ischemic stroke) and cancer (e.g., breast cancer, prostate cancer, ovarian cancer, skin cancer, gastric cancer, glioma, cholangiocarcinoma, bladder cancer, multiple myeloma, colorectal cancer, hepatocellular cancer, cervical cancer, oral squamous cell carcinoma, and/or non-small cell lung cancer (NSCLC)).

In another embodiment, the present disclosure also provides methods for preparing the compounds of structural formula I or formula II, the method comprising: (a) combining in a solvent a substituted anhydride compound of formula III:

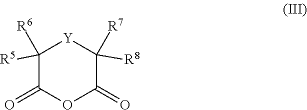

wherein Y, R⁵, R⁶, R⁷ and R⁸ are as defined above;
with a substituted pyridinyl compound of formula IV:

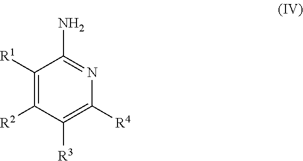

wherein, R¹, R², R³, and R⁴ are as defined above; and
(b) removing the solvent to obtain a compound having the structural formula II.

In at least one embodiment of the methods for preparing the compounds of structural formula I or formula II, the compound of structural formula IV is selected from compound 4a, 4b, 4c, 4d, 4e, 4f, 4g, 4 h, 4i, 4j, 4k, 4l, 4m, 4n, 4o, 4p, 4q, 4r, 4s, 4t, 4u, 4v, 4w, 4x, 4y, 4z, 4aa, 4bb, 4cc, and 4dd (shown in Table 4).

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1A, FIG. 1B, and FIG. 1C depict plots of results showing that inhibitor compounds PD151 and PD152 do not activate transcription by PPARα, PPARγ, or PPARδ. Transcriptional activation assays in COS7 cells co-transfected with vectors encoding either PPARα (FIG. 1A), PPARγ (FIG. 1B), or PPARδ (FIG. 1C), together with a PPAR response element (PPRE), and a vector harboring β-galactosidase, serving as a transfection control. Cells were treated with either one of PPARs' specific agonists Wy-134643 (5 μM), rosiglitazone (5 μM), and GW0742 (5 μM), or one of the compounds PD151 and PD152 (10 μM). Data is mean±SD from 3 independent experiments.

FIG. 2A, FIG. 2B, FIG. 2C, FIG. 2D, FIG. 2E, FIG. 2F, and FIG. 2G depict plots of results showing expression levels of FABP5 in TNBC cell lines and the normal human mammary epithelial cells (HMEC) and that the FABP4/5 inhibitors compound PD151 and PD152 that exhibit specificity for FABP4/5, inhibits growth of TNBC cells in an FABP5-dependent manner but only inhibit proliferation of HMEC at very high dose. Cells in all experiments were treated with one of the denoted compounds at indicated concentrations for 4 days. Cells confluency was measured using Incucyte software. FIG. 2A: Results indicating expression levels of FABP5 in the TNBC lines MB-231 and BT-549, and in HMEC cells. FIG. 2B: Results indicating that compounds PD151 and PD152 at different concentrations inhibit proliferation of MB-231 cell lines. FIG. 2C: IC₅₀ values for MB-231 cells calculated using GraphPad fitting algorithms based on data in FIG. 2B. FIG. 2D: Results indicating that compounds PD151 and PD152 at different concentrations inhibit proliferation of BT-549 cell lines. FIG. 2E: IC₅₀ values for BT-549 cells calculated using GraphPad fitting algorithms based on data in FIG. 2D. FIG. 2F: Results indicating that compound PD152 inhibit proliferation of HMEC cells only in concentration >125 M. FIG. 2G: IC₅₀ values for HMEC cells calculated using GraphPad fitting algorithms based on data in FIG. 2F.

FIG. 3A, FIG. 3B, FIG. 3C, and FIG. 3D depict plots of results indicating that the FABP4/5 inhibitors compounds PD151 and PD152 inhibit proliferation of ovarian cancer cells OVCAR5 and OVCAR8. Cells in all experiments were treated with the denoted compounds at indicated concentrations for 4 days. Cells confluency was measured using Incucyte software. FIG. 3A: Results indicating that compounds PD151 and PD152 inhibit proliferation of OVCAR8 cell line. FIG. 3B: Results indicating that compounds PD151 and PD152 inhibit proliferation of OVCAR5 cell line. FIG. 3C: Results indicating that compounds PD151 and PD152 inhibit proliferation of OVCAR8 cell line in a dose-dependent manner. FIG. 3D: IC50 values for the compounds were calculated using GraphPad fitting algorithms and based on the data in FIG. 3C. Data is mean±SD from 3 independent experiments.

FIG. 4A and FIG. 4B depict results showing that PD152 inhibits lipid uptake in cell culture models of steatosis. FIG. 4A: Plots indicating the intensity of Nile Red staining in hepatic HepG2 cells that were treated with designated concentrations of PD152 for 4 h followed by oleic acid treatment (OA) (1 mM, 24 h). Lipid accumulation was only measured in live cells that were positive for Dapi staining. Quantification of total lipid uptake was measured using Spectra Max i3X plate reader (Molecular Devices) and quantified with the SoftMax Pro 6 software.

FIG. 5A, FIG. 5B, and FIG. 5C depict plots of results of a single-dose pharmacokinetics study following intravenous and oral administration of PD151, PD152, or B4 to male C57BL/6 mice. FIG. 5A: Plots indicating plasma levels of PD151 collected at 0.083, 0.25, 0.5, 1, 2, 4, 6, 8, 24 hours post intravenous administration and 0.25, 0.5, 1, 2, 4, 6, 8, 24 hours post oral administration of the compound the calculated T_{half}, clearance and bioavailability. FIG. 5B: Plots indicating plasma levels of PD152 collected at 0.083, 0.25, 0.5, 1, 2, 4, 6, 8, 24 hours post intravenous administration and 0.25, 0.5, 1, 2, 4, 6, 8, 24 hours post oral administration of the compound the calculated T_{half}, clearance and bioavailability. FIG. 5C: Plots indicating plasma levels of B4 collected at 0.083, 0.25, 0.5, 1, 2, 4, 6, 8, 24 hours post intravenous administration and 0.25, 0.5, 1, 2, 4, 6, 8, 24 hours post oral administration of the compound the calculated T_{half}, clearance and bioavailability.

FIG. 6A and FIG. 6B depict plots of results of a single-dose pharmacokinetics study following intravenous and oral administration of PD152 to male Han Wistar rats and tissue distribution of the compound in male C57BL/6 mice. FIG. 6A: Plots indicating plasma levels of PD152 collected at 0.083, 0.25, 0.5, 1, 2, 4, 6, 8, 24 hours post intravenous administration and 0.25, 0.5, 1, 2, 4, 6, 8, 24 hours post oral administration of the compound the calculated T_{half}, clearance and bioavailability. FIG. 6B: Plots indicating the ratio of plasma/tissue levels of PD152 measured at 0.5, 2, 4, and 8 hours post oral administration at 10 mg/kg.

FIG. 7A and FIG. 7B depict plots of results showing FABP4/5 inhibitor PD152 suppresses tumor growth in vivo in xenograft model. FIG. 7A: Tumor growth in MB-231 xenograft model. MB-231 cells (5×10⁶) were transplanted into the right flank of 7-week-old female Nu/Nu nude mice. Once tumors reach volume of about 50 mm³, treatment started by gavage 5 times a week (20, or 40 mg/kg) or vehicle. Tumor growth was monitored twice a week. Mean±SD (n=5) (by unpaired t-test). FIG. 7B: Data plotted represents the average weight of tumors in each group at the end point (day 30). Statistical significance between the control and treated mice in all experiments was evaluated using a Student's t-test.

FIG. 8A and FIG. 8B depict plots of results showing FABP4/5 inhibitor PD152 ameliorates spontaneous development of autoimmune type I diabetes in NOD mice. FIG. 8A: Blood glucose levels measured once a week starting 8 weeks of age until termination at 27 weeks of age. FIG. 8B: Incidence of diabetes expressed as percentage of all mice in each group at different ages. Diabetes incidence was diagrammed with the Kaplan-Meier method, and incidences between different groups were compared (n=15).

### DETAILED DESCRIPTION

In this specification and in the claims that follow, reference will be made to a number of terms, which shall be defined to have the following meanings:

### General Definitions

In this specification and in the claims that follow, reference will be made to a number of terms, which shall be defined to have the following meanings:

All percentages, ratios and proportions herein are by weight, unless otherwise specified. All temperatures are in degrees Celsius (° C.) unless otherwise specified.

The terms “a” and “an” are defined as one or more unless this disclosure explicitly requires otherwise.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus that “comprises,” “has,” “includes” or “contains” one or more elements possesses those one or more elements but is not limited to possessing only those elements. Likewise, a method that “comprises,” “has,” “includes” or “contains” one or more steps possesses those one or more steps but is not limited to possessing only those one or more steps.

Any embodiment of any of the disclosed methods or compositions can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Any embodiment of any of the disclosed compounds or methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

As used herein, the term “subject” refers to a human or an animal that would benefit from being administered with the FABP3/4/5/7 inhibitor compounds discussed in the present application, such as those suffering from, without limitation a disease affected by expression of one or more of the FABPs, FABP3, FABP4, FABP5, and FABP7, lack of control of free fatty acid serum levels, cancer, metabolic syndrome, or atherosclerosis.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like are encompassed within the term “treating,” and refer to reducing the probability of developing a disorder or condition in a subject, who does not have, but is at risk of or susceptible to developing a disorder or condition.

As used herein, “pharmaceutically acceptable” means physiologically tolerable, for either human or veterinary applications. In addition, “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. Essentially, the pharmaceutically acceptable material is nontoxic to the recipient. The carrier would naturally be selected to minimize any degradation of the active ingredient and to minimize any adverse side effects in the subject, as would be well known to one of skill in the art. For a discussion of pharmaceutically acceptable carriers and other components of pharmaceutical compositions, see, e.g., Remington's Pharmaceutical Sciences, 18th ed., Mack Publishing Company, 1990.

“Test agents” or otherwise “test compounds” as used herein refers to an agent or compound that is to be screened in one or more of the assays described herein. Test agents include compounds of a variety of general types including, but not limited to, small organic molecules, known pharmaceuticals, polypeptides; carbohydrates such as oligosaccharides and polysaccharides; polynucleotides; lipids or phospholipids; fatty acids; steroids; or amino acid analogs. Test agents can be obtained from libraries, such as natural product libraries and combinatorial libraries. In addition, methods of automating assays are known that permit screening of several thousands of compounds in a short period.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, the preferred methods and materials are now described. All publications mentioned herein are incorporated herein by reference to disclose and describe the methods and/or materials in connection with which the publications are cited.

Details associated with the embodiments described above and others are described below.
The herein disclosed aryl, heterocyclic, and heteroaryl units can have one or more hydrogen atoms substituted therefor. Non-limiting examples of substitutions for hydrogen include the following:

Substituted and unsubstituted linear, branched, or cyclic alkyl units include the following non-limiting examples: methyl (C₁), ethyl (C₂), n-propyl (C₃), iso-propyl (C₃), cyclopropyl (C₃), n-butyl (C₄), sec-butyl (C₄), iso-butyl (C₄), tert-butyl (C₄), cyclobutyl (C₄), cyclopentyl (C₅), cyclohexyl (C₆), and the like; whereas substituted linear, branched, or cyclic alkyl, non-limiting examples of which includes, hydroxymethyl (C₁), chloromethyl (C₁), trifluoromethyl (C₁), aminomethyl (C₁), 1-chloroethyl (C₂), 2-hydroxyethyl (C₂), 1,2-difluoroethyl (C₂), 2,2,2-trifluoroethyl (C₃), 3-carboxypropyl (C₃), 2,3-dihydroxycyclobutyl (C₄), and the like.

Substituted and unsubstituted linear, branched, or cyclic alkenyl include, ethenyl (C₂), 3-propenyl (C₃), 1-propenyl (also 2-methylethenyl) (C₃), isopropenyl (also 2-methylethen-2-yl) (C₃), buten-4-yl (C₄), and the like; substituted linear or branched alkenyl, non-limiting examples of which include, 2-chloroethenyl (also 2-chlorovinyl) (C₂), 4-hydroxybuten-1-yl (C₄), 7-hydroxy-7-methyloct-4-en-2-yl (C₉), 7-hydroxy-7-methyloct-3,5-dien-2-yl (C₉), and the like.

Substituted and unsubstituted linear or branched alkynyl include, ethynyl (C₂), prop-2-ynyl (also propargyl) (C₃), propyn-1-yl (C₃), and 2-methyl-hex-4-yn-1-yl (C₇); substituted linear or branched alkynyl, non-limiting examples of which include, 5-hydroxy-5-methylhex-3-ynyl (C₇), 6-hydroxy-6-methylhept-3-yn-2-yl (C₈), 5-hydroxy-5-ethylhept-3-ynyl (C₉), and the like.

Substituted and unsubstituted “alkoxy” are used herein denotes a unit having the general formula —OR¹⁰⁰ wherein R¹⁰⁰ is an alkyl, alkylenyl, or alkynyl unit as defined herein above, for example, methoxy, methoxymethyl, methoxymethyl.

Substituted and unsubstituted “haloalkyl” are used herein denotes an alkyl unit having a hydrogen atom substituted by one or more halogen atoms, for example, trifluoromethyl, 1,2-dicloroethyl, and 3,3,3-trifluoropropyl.

The term “aryl” as used herein denotes cyclic organic units that comprise at least one benzene ring having a conjugated and aromatic six-membered ring, non-limiting examples of which include phenyl (C₆), naphthylen-1-yl (C₁₀), naphthylen-2-yl (C₁₀). Aryl rings can have one or more hydrogen atoms substituted by another organic or inorganic radical. Non-limiting examples of substituted aryl rings include: 4-fluorophenyl (C₆), 2-hydroxyphenyl (C₆), 3-methylphenyl (C₆), 2-amino-4-fluorophenyl (C₆), 2-(N,N-diethylamino)phenyl (C₆), 2-cyanophenyl (C₆), 2,6-di-tert-butylphenyl (C₆), 3-methoxyphenyl (C₆), 8-hydroxynaphthylen-2-yl (C₁₀), 4,5-dimethoxynaphthylen-1-yl (C₁₀), and 6-cyanonaphthylen-1-yl (C₁₀).

The term “heteroaryl” denotes an organic unit comprising a five or six membered conjugated and aromatic ring wherein at least one of the ring atoms is a heteroatom selected from nitrogen, oxygen, or sulfur. The heteroaryl rings can comprise a single ring, for example, a ring having 5 or 6 atoms wherein at least one ring atom is a heteroatom not limited to nitrogen, oxygen, or sulfur, such as a pyridine ring, a furan ring, or thiofuran ring. A “heteroaryl” can also be a fused multicyclic and heteroaromatic ring system having wherein at least one of the rings is an aromatic ring and at least one atom of the aromatic ring is a heteroatom including nitrogen, oxygen, or sulfur. The following are non-limiting examples of heteroaryl rings according to the present disclosure:

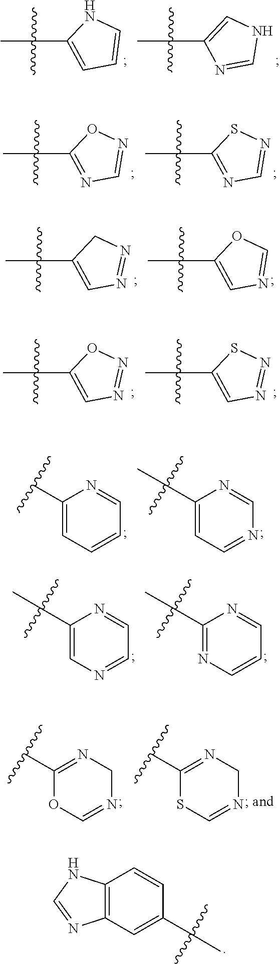

The term “heterocyclic” denotes a ring system having from 3 to 10 atoms wherein at least one of the ring atoms is a heteroatom not limited to nitrogen, oxygen, or sulfur. The rings can be single rings, fused rings, or bicyclic rings. Non-limiting examples of heterocyclic rings include:

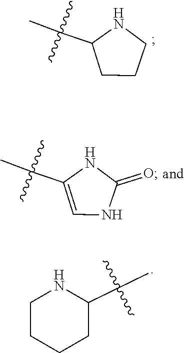 [SMILES: *C1CCCN1 ; *c1c[nH]c(=O)[nH]1 ; *C1CCCCN1]

All of the aforementioned heteroaryl or heterocyclic rings can be optionally substituted with one or more substitutes for hydrogen as described herein further.

Throughout the description of the present disclosure the terms having the spelling “thiophene-2-yl and thiophene-3-yl” are used to describe the heteroaryl units having the respective formulae:

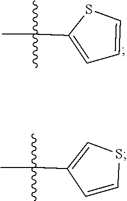 [SMILES: *c1cccs1 ; *c1ccsc1]

whereas in naming the compounds of the present disclosure, the chemical nomenclature for these moieties is typically spelled “thiophen-2-yl and thiophen-3-yl” respectively. Herein the terms “thiophene-2-yl and thiophene-3-yl” are used when describing these rings as units or moieties which make up the compounds of the present disclosure solely to make it unambiguous to the artisan of ordinary skill which rings are referred to herein.

The following are non-limiting examples of units which can substitute for hydrogen atoms on a hydrocarbyl (C₁-C₂₀ linear, branched or cyclic alkyl), aryl, heterocyclic or heteroaryl ring:
i) linear, branched, or cyclic alkyl, alkenyl, and alkynyl; for example, methyl (C₁), ethyl (C₂), n-propyl (C₃), iso-propyl (C₃), cyclopropyl (C₃), propylen-2-yl (C₃), propargyl (C₃), n-butyl (C₄), iso-butyl (C₄), sec-butyl (C₄), tert-butyl (C₄), cyclobutyl (C₄), n-pentyl (C₅), cyclopentyl (C₅), n-hexyl (C₆), and cyclohexyl (C₆);
ii) substituted or unsubstituted aryl; for example, phenyl, 2-fluorophenyl, 3-chlorophenyl, 4-methylphenyl, 2-aminophenyl, 3-hydroxyphenyl, 4-trifluoromethylphenyl, and biphenyl-4-yl;
iii) substituted or unsubstituted heterocyclic; examples of which are provided herein below;
iv) substituted or unsubstituted heteroaryl; examples of which are provided herein below;
v) alkoxy; for example, —OH, —CH₂OH, —OCH₃, —CH₂OCH₃, —OCH₂CH₃, —CH₂OCH₂CH₃, —OCH₂CH₂CH₃, and —CH₂OCH₂CH₂CH₃;
vi) keto; for example, —COCH₃, —CH₂COCH₃, —OCH₂CH₃, —CH₂COCH₂CH₃, —COCH₂CH₂CH₃, and —CH₂COCH₂CH₂CH₃;
vii) alkyl carboxyl; for example, —CO₂CH₃, —CH₂CO₂CH₃, —CO₂CH₂CH₃, —CH₂CO₂CH₂CH₃, —CO₂CH₂CH₂CH₃, and —CH₂CO₂CH₂CH₂CH₃;
viii) alkyl amido; for example, —CONH₂, —CH₂CONH₂, —CONHCH₃, —CH₂CONHCH₃, —CON(CH₃)₂, and —CH₂CON(CH₃)₂;
ix) alkyl carbamate; for example, —OC(O)NH₂, —CH₂OC(O)NH₂, —OC(O)NHCH₃, —CH₂OC(O)NHCH₃, —OC(O)N(CH₃)₂, and —CH₂OC(O)N(CH₃)₂;
x) alkylamino; for example, —NH₂, —CH₂NH₂, —NHCH₃, —N(CH₃)₂, —NH(CH₂CH₃), —CH₂NHCH₃, —CH₂N(CH₃)₂, and —CH₂NH(CH₂CH₃);
xi) halogen: —F, —Cl, —Br, and —I;
xii) —CHₘXₙ; wherein X is halogen, m is from 0 to 2, m+n=3; for example, —CH₂F, —CHF₂, —CF₃, —CCl₃, or —CBr₃;
xiii) alkyl-cyano; for example; —CN, —CH₂CN, and —CH₂CH₂CN;
xiv) alkyl-nitro; for example; —NO₂, —CH₂NO₂, and —CH₂CH₂NO₂;
xv) alkylenesulfonyl alkyl; for example, —SO₂H, —CH₂SO₂H, —SO₂CH₃, —CH₂SO₂CH₃, —SO₂C₆H₅, and —CH₂SO₂C₆H₅; xvi) alkylene sulfonic acid; for example, —SO₃H, —CH₂SO₃H;
xvii) hydroxyl groups or thiol groups, or
xviii) amino groups, monosubstituted amino, or disubstituted amino.

For the purposes of the present disclosure the terms “compound,” “analog,” and “composition of matter” stand equally well for the HIF-1α prolyl hydroxylase enzyme inhibitors described herein, including all enantiomeric forms, diastereomeric forms, salts, and the like, and the terms “compound,” “analog,” and “composition of matter” are used interchangeably throughout the present specification.

The compounds disclosed herein include all salt forms, for example, salts of both basic groups, inter alia, amines, as well as salts of acidic groups, inter alia, carboxylic acids. The following are non-limiting examples of anions that can form pharmaceutically acceptable salts with basic groups: chloride, bromide, iodide, sulfate, bisulfate, carbonate, bicarbonate, phosphate, formate, acetate, propionate, butyrate, pyruvate, lactate, oxalate, malonate, maleate, succinate, tartrate, fumarate, citrate, and the like. The following are non-limiting examples of cations that can form pharmaceutically acceptable salts of the anionic form of acidic substituent groups on the compounds described herein: sodium, lithium, potassium, calcium, magnesium, zinc, bismuth, and the like.

### FABP3/4/5/7 Inhibitor Compounds

Disclosed herein are compounds that inhibit one or more of the FABP3, FABP4, FABP5, and FABP7 (i.e., an “FABP3/4/5/7 inhibitor”). These FABP3/4/5/7 inhibitor compounds of the present disclosure are based on a substituted 2-amino pyridinyl ring having the general structural formula I:

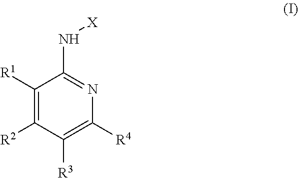

wherein,
   R¹, R², R³, and R⁴ is each independently selected from hydrogen or a substitution for hydrogen;
   X is a moiety of formula:

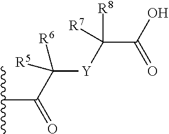

wherein,
   Y is a heteroatom selected from —S—, —O—, —SO—, and —SO₂—, or is —CR⁹R¹⁰—, wherein R⁹ and R¹⁰ are each independently selected from hydrogen, C₁-C₄ linear or branched alkyl, phenyl, and benzyl, or R⁹ and R¹⁰ together form a cyclopropyl, cyclobutyl, or cyclopentyl ring;
   R⁵, R⁶, R⁷, and R⁸ is each independently selected from hydrogen, C₁-C₄ linear or branched alkyl, phenyl, and benzyl, and/or
   R⁵ and R⁶ together or R⁷ and R⁸ together form a cyclopropyl ring, a cyclobutyl ring, a cyclopentyl ring, or an oxetane ring,
   R⁶ and R⁷ together form a 4-, 5-, or 6-membered carbocyclic or heterocyclic ring with Y as a member of the ring, or
   R⁵ and R⁶ together and R⁷ and R⁸ together form a bicyclo[1.1.1] ring.

In at least one embodiment, the compound of structural formula I includes any pharmaceutically acceptable salt thereof.

In at least one embodiment of the FABP3/4/5/7 inhibitor compound of structural formula I, R¹, R², R³, and R⁴ is each independently selected from: hydrogen; halogen; C₁-C₄ linear or branched, saturated or unsaturated alkyl; C₁-C₄ linear or branched, saturated or unsaturated alkoxy; —(CR^{12a}R^{12b})_{q}OR¹³; —(CR^{12a}R^{12b})_{q}C(O)R¹³; —(CR^{12a}R^{12b})_{q}C(O)OR¹³; —O(CR^{12a}R^{12b})_{q}C(O)OR¹³; —(CR^{12a}R^{12b})_{q}N(R¹³)₂; —CHₘXₙ; —(CR^{12a}R^{12b})_{q}CN; and —(CR^{12a}R^{12b})_{q}NO₂; wherein, R¹³ is independently hydrogen, C₁-C₄ linear or branched alkyl, phenyl, or benzyl; and wherein R^{12a} and R^{12b} are each independently hydrogen, methyl (C₁), or ethyl (C₂) and the index q is an integer from 0 to 4, X is halogen, m is 0 to 2, and m+n=3.

In at least one embodiment of the FABP3/4/5/7 inhibitor compound of structural formula I, R¹, R², R³, and R⁴ is each independently selected from: hydrogen; halogen; C₁-C₄ linear, branched, or cyclic, saturated or unsaturated alkyl, phenyl, benzyl, substituted or unsubstituted C₁-C₄ linear, branched, or cyclic, saturated or unsaturated alkoxy, phenoxy, or benzyloxy.

In at least one embodiment of the FABP3/4/5/7 inhibitor compound of structural formula I, R¹, R², R³, and R⁴ is each independently selected from hydrogen, chlorine, fluorine, tri-fluoromethyl, methoxy, dichloro-methoxy, difluoro-methoxy, trichloro-methoxy, trifluoro-methoxy, ethoxy, propoxy, isopropyloxy, cyclopropyloxy, propargyloxy, butoxy, iso-butoxy, sec-butoxy, tert-butoxy, 2-methoxyethoxy, 2-ethoxy-2-oxoethoxy, (3-methyloxetan-3-yl)oxy, or benzyloxy.

In at least one embodiment of the FABP3/4/5/7 inhibitor compound of structural formula I,
R¹ is selected from chlorine, fluorine, methyl, and tri-fluoromethyl;
R² is hydrogen;
R³ is selected from hydrogen, chlorine, fluorine, methyl, and tri-fluoromethyl; and
R⁴ is each independently selected from methoxy, dichloro-methoxy, difluoro-methoxy, trichloro-methoxy, trifluoro-methoxy, ethoxy, propoxy, isopropoxy, cyclopropoxy, propargyloxy, butoxy, iso-butoxy, sec-butoxy, tert-butoxy, 2-methoxyethoxy, 2-ethoxy-2-oxoethoxy, (3-methyloxetan-3-yl)oxy, or benzyloxy.

In at least one embodiment of the FABP3/4/5/7 inhibitor compound of structural formula I, the compound of structural formula I has a structural formula selected from Ia, Ib, Ic, Id, Ie, If, Ig, Ih, Ii, Ij, Ik, Il, Im, In, Io, Ip, Iq, Ir, Is, It, Iu, Iv, Iw, Ix, Iy, Iz, Iaa, Ibb, Icc, and Idd as shown below in Table 1.

TABLE 1

| | |
|---|---|
| 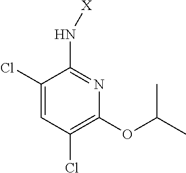 | Ia |
| 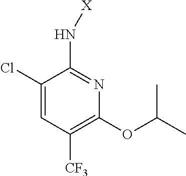 | Ib |
| 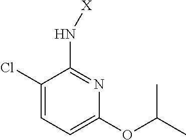 | Ic |
| 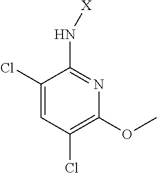 | Id |
| 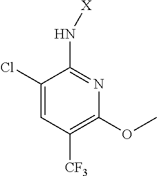 | Ie |
| 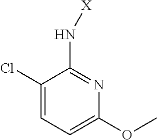 | If |
| 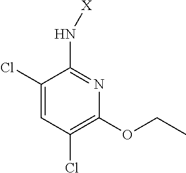 | Ig |
| 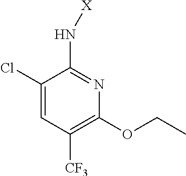 | Ih |
| 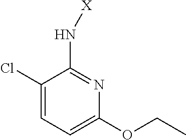 | Ii |
| 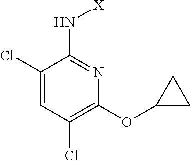 | Ij |
| 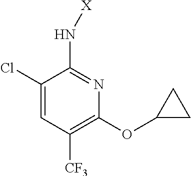 | Ik |
| 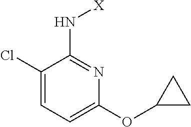 | Il |
| 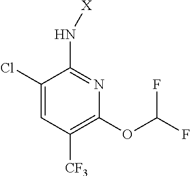 | Im |
| 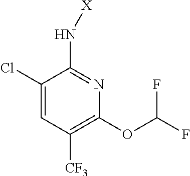 | In |
| 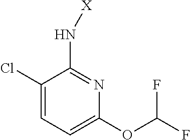 | Io |
| 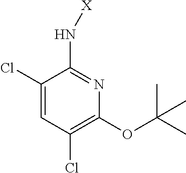 | Ip |
| 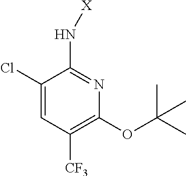 | Iq |
| 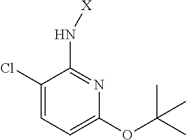 | Ir |
| 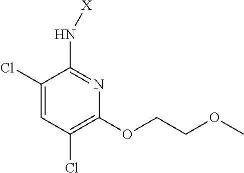 | Is |
| 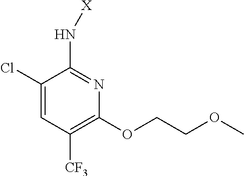 | It |
| 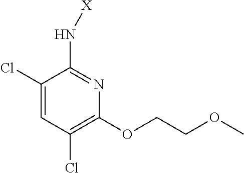 | Iu |
| 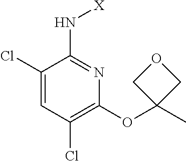 | Iv |
| 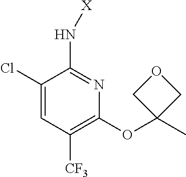 | Iw |
| 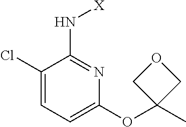 | Ix |
| 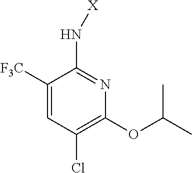 | Iy |
| 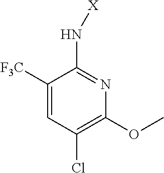 | Iz |
| 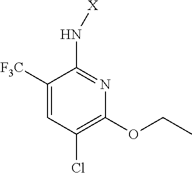 | Iaa |
| 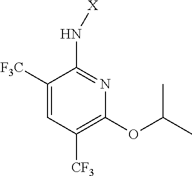 | Ibb |
| 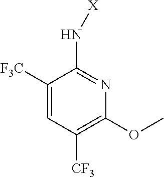 | Icc |
| 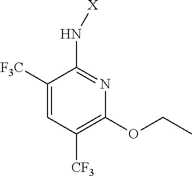 | Idd |

The FABP3/4/5/7 inhibitor compounds having structural formula I of the present disclosure comprise a moiety X attached to the amine group of the substituted pyridinyl group. The X moiety is a chemical group of formula:

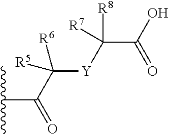

wherein,
   Y is a heteroatom selected from —S—, —O—, —SO—, and —SO₂—, or is —CR⁹R¹⁰—, wherein R⁹ and R¹⁰ are each independently selected from hydrogen, C₁-C₄ linear or branched alkyl, phenyl, and benzyl, or R⁹ and R¹⁰ together form a cyclopropyl, cyclobutyl, or cyclopentyl ring;
   R⁵, R⁶, R⁷, and R⁸ is each independently selected from hydrogen, C₁-C₄ linear or branched alkyl, phenyl, and benzyl, and/or
   R⁵ and R⁶ together or R⁷ and R⁸ together form a cyclopropyl ring, a cyclobutyl ring, a cyclopentyl ring, or an oxetane ring,
   R⁶ and R⁷ together form a 4-, 5-, or 6-membered carbocyclic or heterocyclic ring with Y as a member of the ring, or
   R⁵ and R⁶ together and R⁷ and R⁸ together form a bicyclo[1.1.1] ring.

In at least one embodiment of the FABP3/4/5/7 inhibitor compound of structural formula I, Y is selected from —S— or —O— and R⁵, R⁶, R⁷, and R⁸ are each independently hydrogen or C₁-C₄ linear or branched alkyl. In at least one embodiment of the FABP3/4/5/7 inhibitor compound of structural formula I, R⁵ and R⁶ are hydrogen; and R⁷ and R⁸ are methyl, or R⁷ and R⁸ together form a cyclopropyl ring, cyclobutyl ring, cyclopentyl ring, or oxetane ring.

Exemplary X moieties where Y is a sulfur atom (—S—), an —SO—, or an —SO₂— can include any of the moieties shown below:

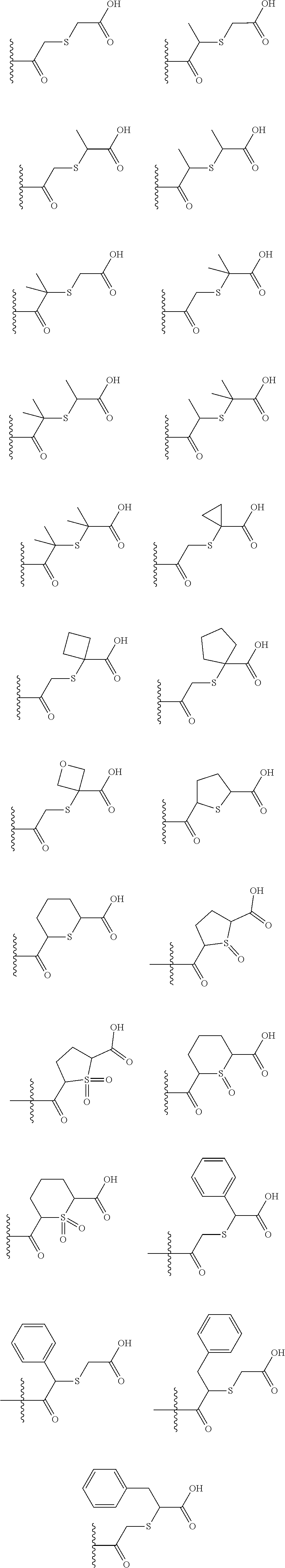

Exemplary X moieties where Y is an oxygen atom (—O—) can include any of the moieties shown below:

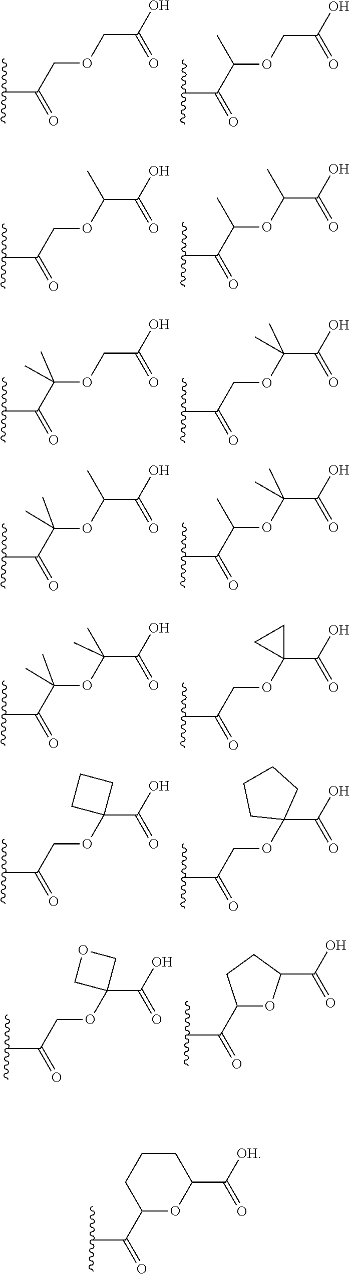

In at least one embodiment of the compounds of structural formula I, the chemical group Y of the X moiety is —CR⁹R¹⁰—, wherein R⁹ and R¹⁰ are each independently selected from hydrogen, C₁-C₄ linear or branched alkyl, phenyl, and benzyl, or R⁹ and R¹⁰ together form a cyclopropyl, cyclobutyl, or cyclopentyl ring, and the chemical groups at positions R⁵, R⁶, R⁷ and R⁸ are each independently hydrogen, or C₁-C₄ linear or branched alkyl, or R⁵ and R⁶ are hydrogen; and R⁷ and R⁸ are methyl, or R and R′ together form a cyclopropyl ring, cyclobutyl ring, cyclopentyl ring, or oxetane ring.

Exemplary X moieties where Y is —CR⁹R¹⁰— can include any of the moieties shown below:

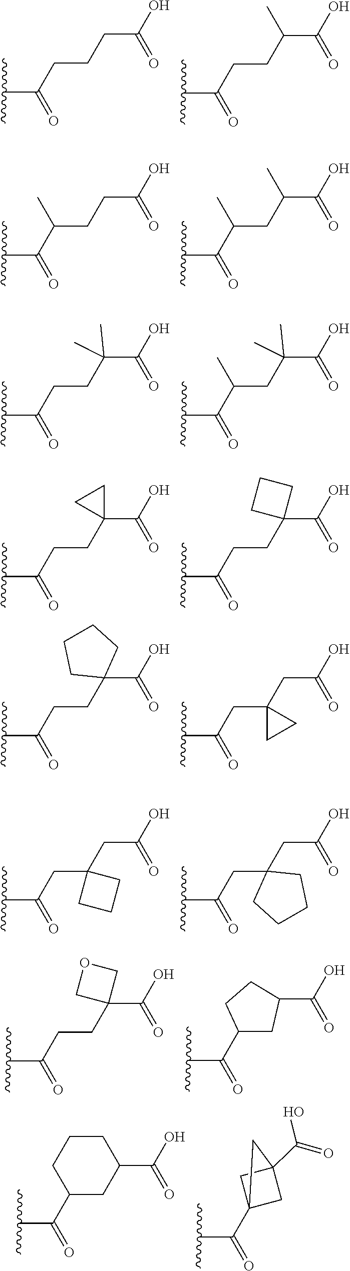

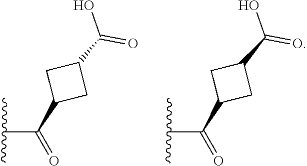

The various inhibitor compounds of structural formula I provided in the present disclosure include a range of compounds with various substituted 2-amino-pyridinyl ring moieties combined with various X moieties. The various FABP3/4/5/7 inhibitor compounds can be represented as compounds of structural formula II and any pharmaceutically acceptable salt thereof:

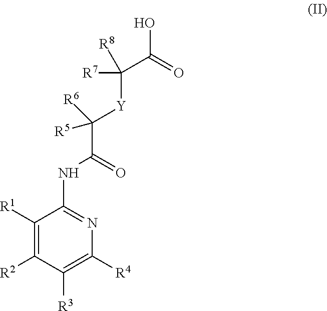

wherein,
   Y is a heteroatom selected from —S—, —O—, —SO—, and —SO₂—, or is —CR⁹R¹⁰—, wherein R⁹ and R¹⁰ are each independently selected from hydrogen, C₁-C₄ linear or branched alkyl, phenyl, and benzyl, or R⁹ and R¹⁰ together form a cyclopropyl, cyclobutyl, or cyclopentyl ring;
   R⁵, R⁶, R⁷, and R⁸ is each independently selected from hydrogen, C₁-C₄ linear or branched alkyl, phenyl, and benzyl, and/or
   R⁵ and R⁶ together or R⁷ and R⁸ together form a cyclopropyl ring, a cyclobutyl ring, a cyclopentyl ring, or an oxetane ring,
   R⁶ and R⁷ together form a 4-, 5-, or 6-membered carbocyclic or heterocyclic ring with Y as a member of the ring, or
   R⁵ and R⁶ together and R⁷ and R⁸ together form a bicyclo[1.1.1] ring.

In at least one embodiment of the FABP3/4/5/7 inhibitor compound of structural formula II, R¹, R², R³, and R⁴ is each independently selected from: hydrogen; halogen; C₁-C₄ linear or branched, saturated or unsaturated alkyl; C₁-C₄ linear or branched, saturated or unsaturated alkoxy; —(CR^{12a}R^{12b})_{q}OR¹³; —(CR^{12a}R^{12b})_{q}C(O)R¹³; —(CR^{12a}R^{12b})_{q}C(O)OR¹³; —O(CR^{12a}R^{12b})_{q}C(O)OR¹³; —(CR^{12a}R^{12b})_{q}N(R¹³)₂; —CHₘXₙ; —(CR^{12a}R^{12b})_{q}CN; and —(CR^{12a}R^{12b})_{q}NO₂; wherein, R¹³ is independently hydrogen, C₁-C₄ linear or branched alkyl, phenyl, or benzyl; and wherein R^{12a} and R^{12b} are each independently hydrogen, methyl (C₁), or ethyl (C₂) and the index q is an integer from 0 to 4, X is halogen, m is 0 to 2, and m+n=3.

In at least one embodiment of the FABP3/4/5/7 inhibitor compound of structural formula II, R¹, R², R³, and R⁴ is each independently selected from: hydrogen; halogen; C₁-C₄ linear, branched, or cyclic, saturated or unsaturated alkyl, phenyl, benzyl, substituted or unsubstituted C₁-C₄ linear, branched, or cyclic, saturated or unsaturated alkoxy, phenoxy, or benzyloxy.

In at least one embodiment of the FABP3/4/5/7 inhibitor compound of structural formula II, R¹, R², R³, and R⁴ is each independently selected from hydrogen, chlorine, fluorine, tri-fluoromethyl, methoxy, dichloro-methoxy, difluoro-methoxy, trichloro-methoxy, trifluoro-methoxy, ethoxy, propoxy, isopropyloxy, cyclopropyloxy, propargyloxy, butoxy, iso-butoxy, sec-butoxy, tert-butoxy, 2-methoxyethoxy, 2-ethoxy-2-oxoethoxy, (3-methyloxetan-3-yl)oxy, or benzyloxy.

In at least one embodiment of the FABP3/4/5/7 inhibitor compound of structural formula II,
R¹ is selected from chlorine, fluorine, methyl, and tri-fluoromethyl;
R² is hydrogen;
R³ is selected from hydrogen, chlorine, fluorine, methyl, and tri-fluoromethyl; and
R⁴ is each independently selected from methoxy, dichloro-methoxy, difluoro-methoxy, trichloro-methoxy, trifluoro-methoxy, ethoxy, propoxy, isopropoxy, cyclopropoxy, propargyloxy, butoxy, iso-butoxy, sec-butoxy, tert-butoxy, 2-methoxyethoxy, 2-ethoxy-2-oxoethoxy, (3-methyloxetan-3-yl)oxy, or benzyloxy.

In at least one embodiment of the FABP3/4/5/7 inhibitor compound of structural formula II, the compound of structural formula II has a structural formula selected from IIa, IIb, IIc, IId, IIe, IIf, IIg, IIh, IIi, IIj, IIk, IID, IIm, IIn, IIo, IIp, IIq, IIr, IIs, IIt, IIu, IIv, IIw, IIx, IIy, IIz, IIaa, IIbb, IIcc, and IIdd, shown below in Table 2.

TABLE 2

| | |
|---|---|
| 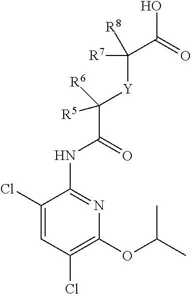 | IIa |
| 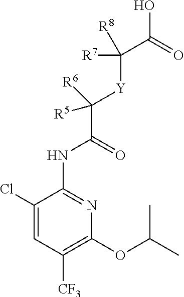 | IIb |
| 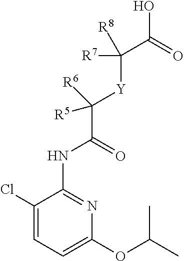 | IIc |
| 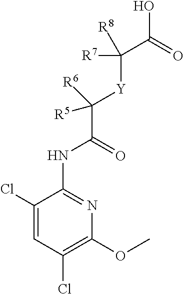 | IId |
| 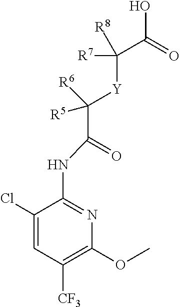 | IIe |
| 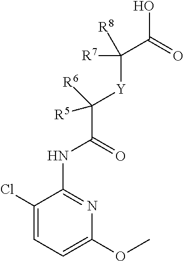 | IIf |
| 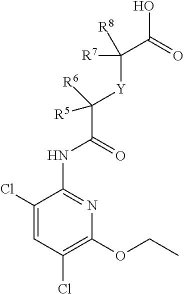 | IIg |
| 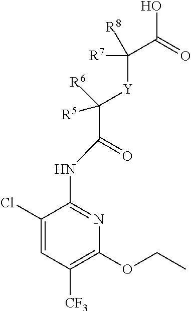 | IIh |
| 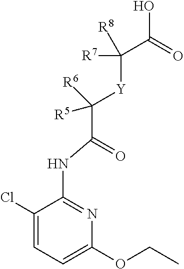 | IIi |
| 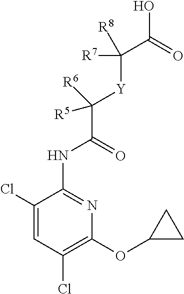 | IIj |
| 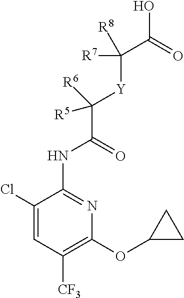 | IIk |
| 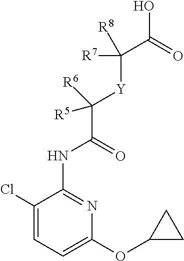 | IIl |
| 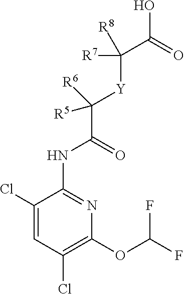 | IIm |
| 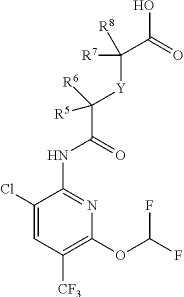 | IIn |
| 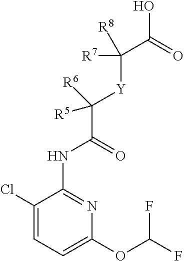 | IIo |
| 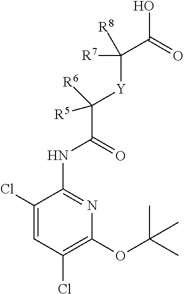 | IIp |
| 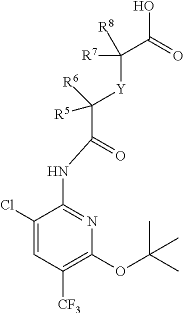 | IIq |
| 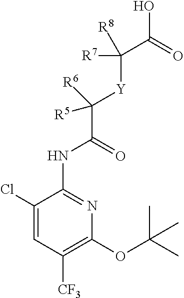 | IIr |
| 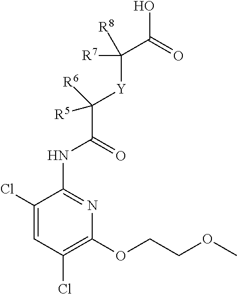 | IIs |
| 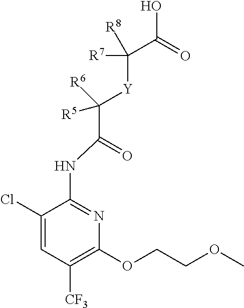 | IIt |
| 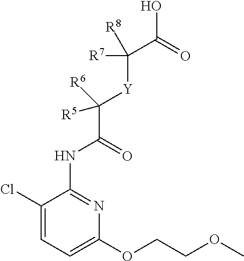 | IIu |
| 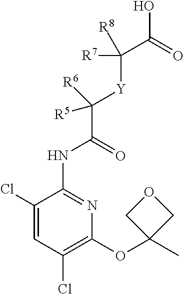 | IIv |
| 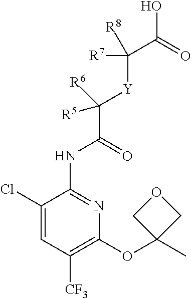 | IIw |
| 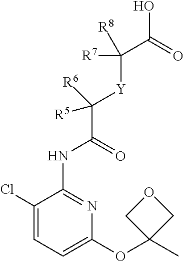 | IIx |
| 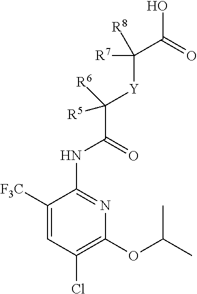 | IIy |
| 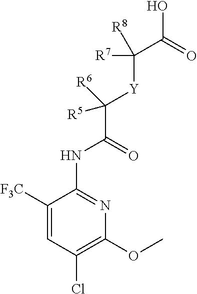 | IIz |
| 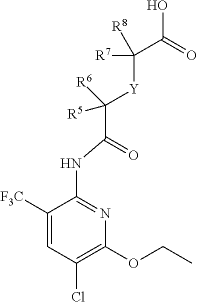 | IIaa |
| 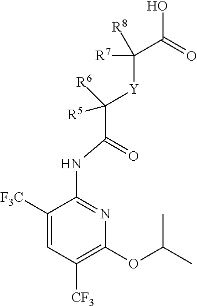 | IIbb |
| 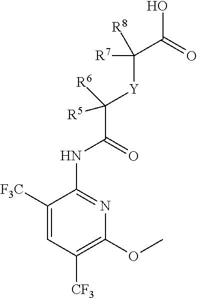 | IIcc |
| 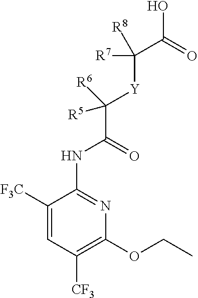 | IIdd. |

In at least one embodiment of the compound of structural formula I and/or the compound of structural formula II, the compound is selected from any of compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60, shown in Table 3 below.

TABLE 3

| | |
|---|---|
| 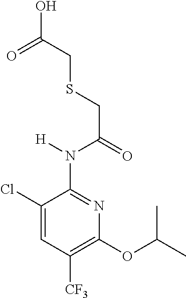 [SMILES: CC(C)Oc1nc(NC(=O)CSCC(=O)O)c(Cl)cc1C(F)(F)F] | 1 |
| 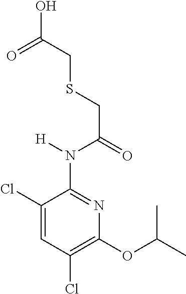 [SMILES: CC(C)Oc1nc(NC(=O)CSCC(=O)O)c(Cl)cc1Cl] | 2 |
| 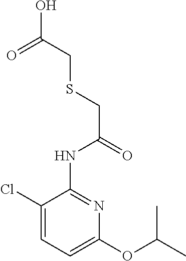 [SMILES: CC(C)Oc1ccc(Cl)c(NC(=O)CSCC(=O)O)n1] | 3 |
| 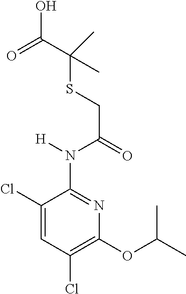 [SMILES: CC(C)Oc1nc(NC(=O)CSC(C)(C)C(=O)O)c(Cl)cc1Cl] | 4 |
| 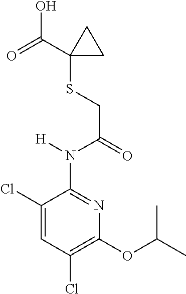 [SMILES: CC(C)Oc1nc(NC(=O)CSC2(C(=O)O)CC2)c(Cl)cc1Cl] | 5 |
| 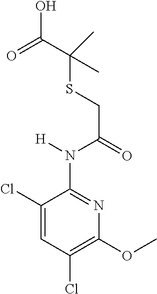 [SMILES: COc1nc(NC(=O)CSC(C)(C)C(=O)O)c(Cl)cc1Cl] | 6 |
| 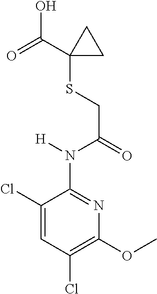 [SMILES: COc1nc(NC(=O)CSC2(C(=O)O)CC2)c(Cl)cc1Cl] | 7 |
| 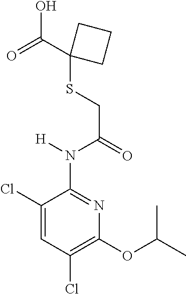 [SMILES: CC(C)Oc1nc(NC(=O)CSC2(C(=O)O)CCC2)c(Cl)cc1Cl] | 8 |
| 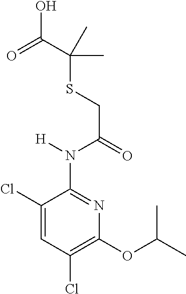 [SMILES: CC(C)Oc1nc(NC(=O)CSC(C)(C)C(=O)O)c(Cl)cc1Cl] | 9 |
| 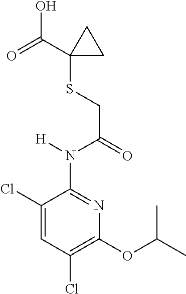 [SMILES: CC(C)Oc1nc(NC(=O)CSC2(C(=O)O)CC2)c(Cl)cc1Cl] | 10 |
| 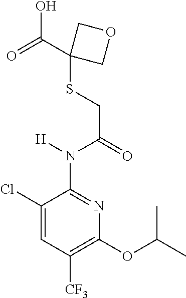 [SMILES: CC(C)Oc1nc(NC(=O)CSC2(C(=O)O)COC2)c(Cl)cc1C(F)(F)F] | 11 |
| 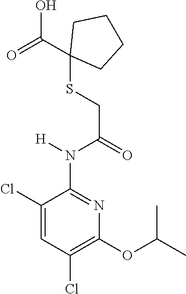 [SMILES: CC(C)Oc1nc(NC(=O)CSC2(C(=O)O)CCCC2)c(Cl)cc1Cl] | 12 |
| 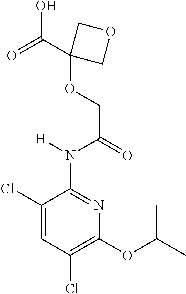 [SMILES: CC(C)Oc1nc(NC(=O)COC2(C(=O)O)COC2)c(Cl)cc1Cl] | 14 |
| 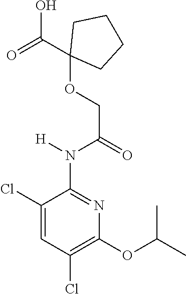 [SMILES: CC(C)Oc1nc(NC(=O)COC2(C(=O)O)CCCC2)c(Cl)cc1Cl] | 15 |
| 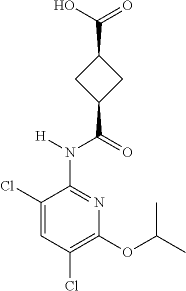 [SMILES: CC(C)Oc1nc(NC(=O)[C@H]2C[C@@H](C(=O)O)C2)c(Cl)cc1Cl] | 16 |
| 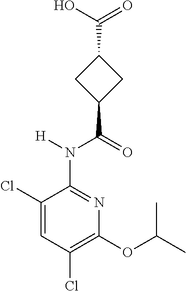 [SMILES: CC(C)Oc1nc(NC(=O)[C@H]2C[C@H](C(=O)O)C2)c(Cl)cc1Cl] | 17 |
| 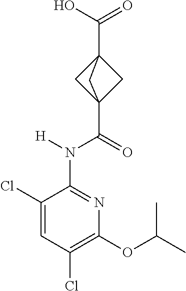 [SMILES: CC(C)Oc1nc(NC(=O)C23CC(C(=O)O)(C2)C3)c(Cl)cc1Cl] | 18 |
| 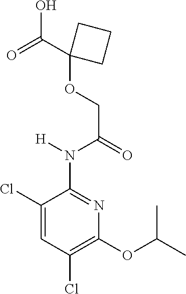 [SMILES: CC(C)Oc1nc(NC(=O)COC2(C(=O)O)CCC2)c(Cl)cc1Cl] | 19 |
| 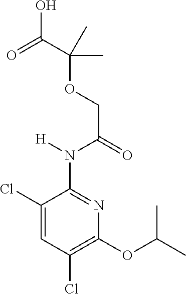 [SMILES: CC(C)Oc1nc(NC(=O)COC(C)(C)C(=O)O)c(Cl)cc1Cl] | 20 |
| 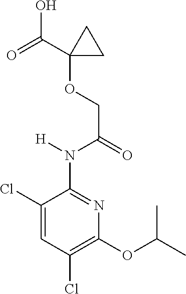 [SMILES: CC(C)Oc1nc(NC(=O)COC2(C(=O)O)CC2)c(Cl)cc1Cl] | 21 |
| 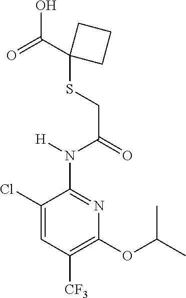 [SMILES: CC(C)Oc1nc(NC(=O)CSC2(C(=O)O)CCC2)c(Cl)cc1C(F)(F)F] | 22 |
| 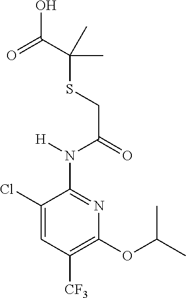 [SMILES: CC(C)Oc1nc(NC(=O)CSC(C)(C)C(=O)O)c(Cl)cc1C(F)(F)F] | 23 |
| 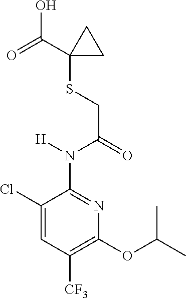 [SMILES: CC(C)Oc1nc(NC(=O)CSC2(C(=O)O)CC2)c(Cl)cc1C(F)(F)F] | 24 |
| 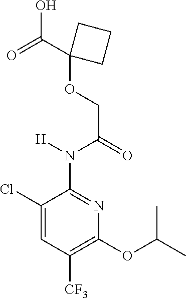 [SMILES: CC(C)Oc1nc(NC(=O)COC2(C(=O)O)CCC2)c(Cl)cc1C(F)(F)F] | 25 |
| 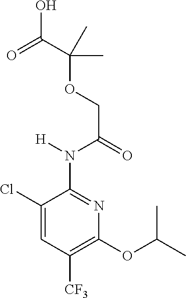 [SMILES: CC(C)Oc1nc(NC(=O)COC(C)(C)C(=O)O)c(Cl)cc1C(F)(F)F] | 26 |
| 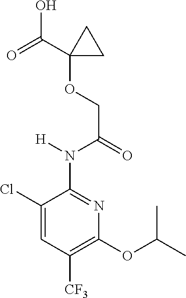 [SMILES: CC(C)Oc1nc(NC(=O)COC2(C(=O)O)CC2)c(Cl)cc1C(F)(F)F] | 27 |
| 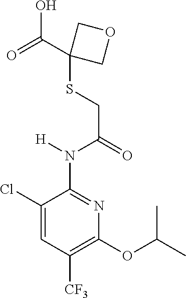 [SMILES: CC(C)Oc1nc(NC(=O)CSC2(C(=O)O)COC2)c(Cl)cc1C(F)(F)F] | 28 |
| 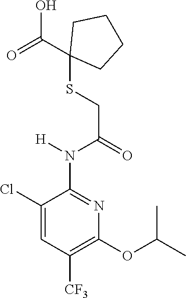 [SMILES: CC(C)Oc1nc(NC(=O)CSC2(C(=O)O)CCCC2)c(Cl)cc1C(F)(F)F] | 29 |
| 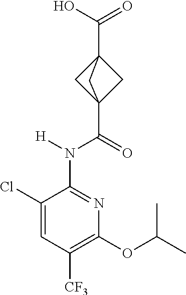 [SMILES: CC(C)Oc1nc(NC(=O)C23CC(C(=O)O)(C2)C3)c(Cl)cc1C(F)(F)F] | 30 |
| 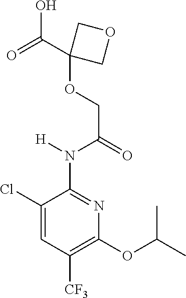 [SMILES: CC(C)Oc1nc(NC(=O)COC2(C(=O)O)COC2)c(Cl)cc1C(F)(F)F] | 31 |
| 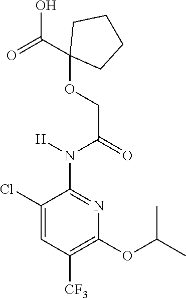 [SMILES: CC(C)Oc1nc(NC(=O)COC2(C(=O)O)CCCC2)c(Cl)cc1C(F)(F)F] | 32 |
| 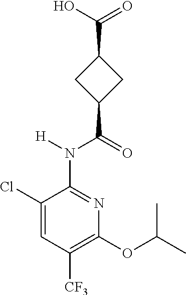 [SMILES: CC(C)Oc1nc(NC(=O)[C@H]2C[C@@H](C(=O)O)C2)c(Cl)cc1C(F)(F)F] | 33 |
| 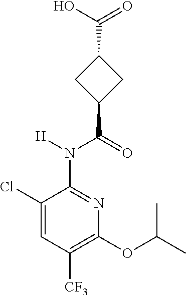 [SMILES: CC(C)Oc1nc(NC(=O)[C@H]2C[C@H](C(=O)O)C2)c(Cl)cc1C(F)(F)F] | 34 |
| 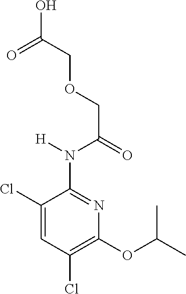 [SMILES: CC(C)Oc1nc(NC(=O)COCC(=O)O)c(Cl)cc1Cl] | 35 |
| 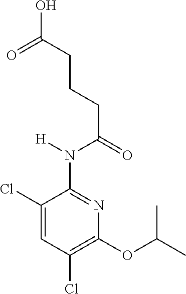 [SMILES: CC(C)Oc1nc(NC(=O)CCCC(=O)O)c(Cl)cc1Cl] | 36 |
| 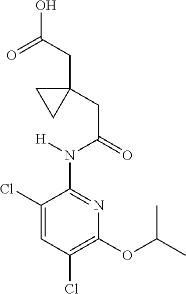 [SMILES: CC(C)Oc1nc(NC(=O)CC2(CC(=O)O)CC2)c(Cl)cc1Cl] | 38 |
| 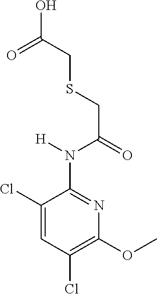 [SMILES: COc1nc(NC(=O)CSCC(=O)O)c(Cl)cc1Cl] | 39 |
| 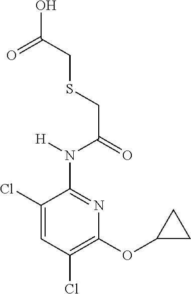 [SMILES: O=C(O)CSCC(=O)Nc1nc(OC2CC2)c(Cl)cc1Cl] | 40 |
| 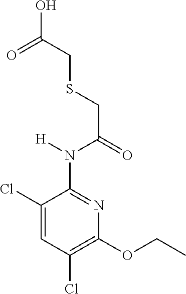 [SMILES: CCOc1nc(NC(=O)CSCC(=O)O)c(Cl)cc1Cl] | 41 |
| 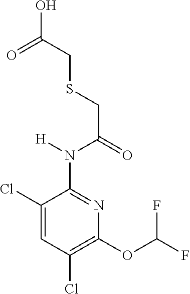 [SMILES: O=C(O)CSCC(=O)Nc1nc(OC(F)F)c(Cl)cc1Cl] | 42 |
| 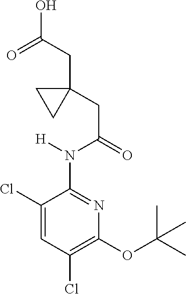 [SMILES: CC(C)(C)Oc1nc(NC(=O)CC2(CC(=O)O)CC2)c(Cl)cc1Cl] | 43 |
| 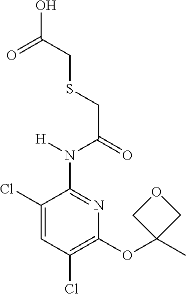 [SMILES: CC1(Oc2nc(NC(=O)CSCC(=O)O)c(Cl)cc2Cl)COC1] | 44 |
| 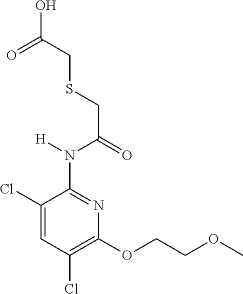 [SMILES: COCCOc1nc(NC(=O)CSCC(=O)O)c(Cl)cc1Cl] | 46 |
| 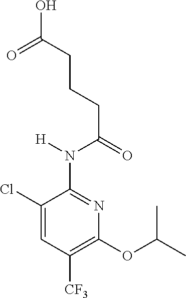 [SMILES: CC(C)Oc1nc(NC(=O)CCCC(=O)O)c(Cl)cc1C(F)(F)F] | 47 |
| 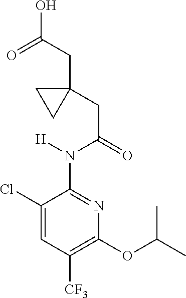 [SMILES: CC(C)Oc1nc(NC(=O)CC2(CC(=O)O)CC2)c(Cl)cc1C(F)(F)F] | 49 |
| 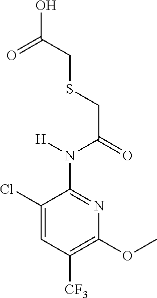 [SMILES: COc1nc(NC(=O)CSCC(=O)O)c(Cl)cc1C(F)(F)F] | 50 |
| 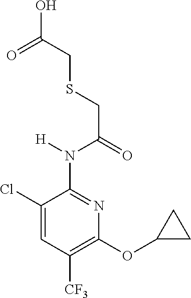 [SMILES: O=C(O)CSCC(=O)Nc1nc(OC2CC2)c(C(F)(F)F)cc1Cl] | 51 |
| 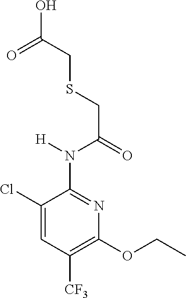 [SMILES: CCOc1nc(NC(=O)CSCC(=O)O)c(Cl)cc1C(F)(F)F] | 52 |
| 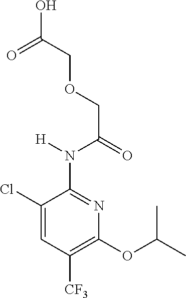 [SMILES: CC(C)Oc1nc(NC(=O)COCC(=O)O)c(Cl)cc1C(F)(F)F] | 53 |
| 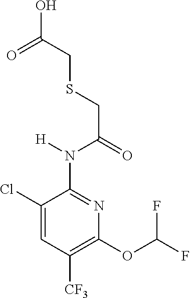 [SMILES: O=C(O)CSCC(=O)Nc1nc(OC(F)F)c(C(F)(F)F)cc1Cl] | 54 |
| 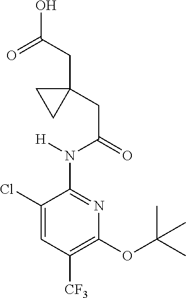 [SMILES: CC(C)(C)Oc1nc(NC(=O)CC2(CC(=O)O)CC2)c(Cl)cc1C(F)(F)F] | 55 |
| 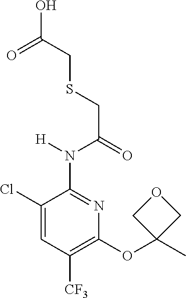 [SMILES: CC1(Oc2nc(NC(=O)CSCC(=O)O)c(Cl)cc2C(F)(F)F)COC1] | 56 |
| 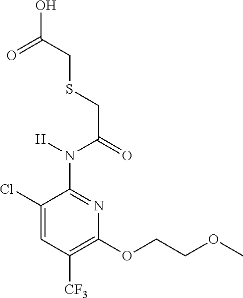 [SMILES: COCCOc1nc(NC(=O)CSCC(=O)O)c(Cl)cc1C(F)(F)F] | 57 |
| 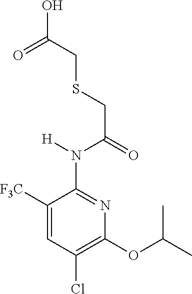 [SMILES: CC(C)Oc1nc(NC(=O)CSCC(=O)O)c(C(F)(F)F)cc1Cl] | 58 |
| 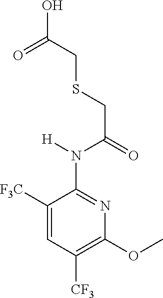 [SMILES: COc1nc(NC(=O)CSCC(=O)O)c(C(F)(F)F)cc1C(F)(F)F] | 59. |
| 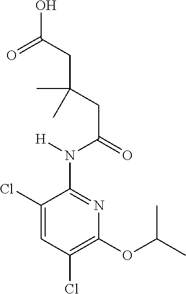 [SMILES: CC(C)Oc1nc(NC(=O)CC(C)(C)CC(=O)O)c(Cl)cc1Cl] | 60. |

As described elsewhere herein, one of ordinary skill will understand that the FABP3/4/5/7 compounds provided herein can exist in various well-known closely-related and/or equivalent forms not explicitly described by the chemical structures and formulae. It is intended that the FABP3/4/5/7 inhibitor compounds of structural formula I and II of the present disclosure (including the compounds of Table 3, and as described the Examples) includes these closely-related forms of the compounds defined by the chemical structures and formulae including, but not limited to, pharmaceutically acceptable salts of the compounds, mixture of stereoisomers of the compounds, single stereoisomers of the compounds, tautomeric forms of the compounds, and/or prodrug forms of the compounds.

### Preparation of FABP3/4/5/7 Inhibitor Compounds

The present disclosure also provides processes for preparing the FABP3/4/5/7 inhibitor compounds disclosed herein, including the compounds of structural formula I and II (as defined elsewhere herein), which is outlined generally in Scheme A, and described in greater detail below.

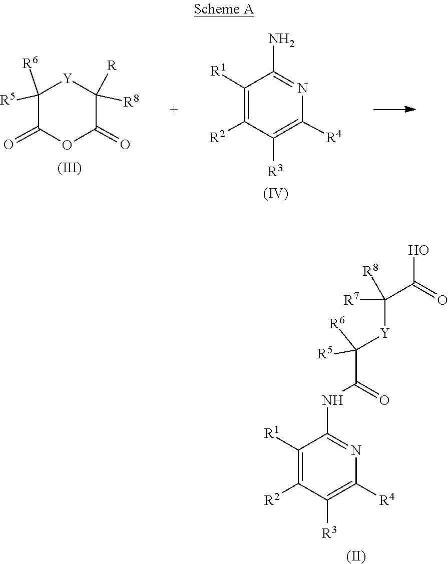

A mixture of the substituted anhydride compound of structural formula III, and the substituted 2-amino-pyridinyl compound of structural formula IV, (1:0.75 molar ratio) is purged with argon then dissolved in a dry solvent. The reaction mixture is then stirred at a temperature from room temperature to the reflux temperature of the chosen solvent. The progress of the reaction can be followed by one or more analytical methods, for example, thin layer chromatography (TLC), gas chromatography, and the like. After the starting material 2-amino-pyridinyl IV, or substituted anhydride, III, is deemed to be consumed, the solvent is removed in vacuo to afford the desired FABP3/4/5/7 inhibitor of structural formula II.

In at least one embodiment, the process for preparing the disclosed FABP3/4/5/7 inhibitor compounds of structural formula II, comprising:
(a) combining in a solvent a substituted anhydride compound of formula III:

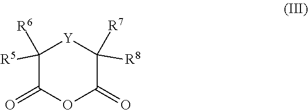

wherein Y is a heteroatom selected from —S—, —O—, —SO—, and —SO₂—, or is —CR⁹R¹⁰—, wherein R⁹ and R¹⁰ are each independently selected from hydrogen, C₁-C₄ linear or branched alkyl, phenyl, and benzyl, or R⁹ and R¹⁰ together form a cyclopropyl, cyclobutyl, or cyclopentyl ring; R⁵, R⁶, R⁷ and R⁸ is each independently selected from hydrogen, C₁-C₄ linear or branched alkyl, phenyl, and benzyl, and/or R⁵ and R⁶ together or R⁷ and R⁸ together form a cyclopropyl ring, a cyclobutyl ring, a cyclopentyl ring, or an oxetane ring, or R⁶ and R⁷ together form a 4-, 5-, or 6-membered carbocyclic or heterocyclic ring with Y as a member of the ring, or R⁵ and R⁶ together and R⁷ and R⁸ together form a bicyclo[1.1.1] ring;
with a substituted 2-amino-pyridinyl compound of formula IV:

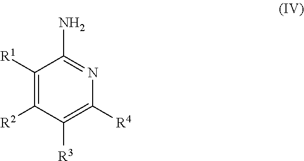

wherein, R¹ is selected from hydrogen, cyano, and 5-membered heteroaryl ring; and R² and R³ are each independently selected from hydrogen, C₁-C₄ linear or branched alkyl, cyclopropyl, cyclobutyl, phenyl, and benzyl, or R² and R³ together form a 5- to 6-membered carbocyclic or heterocyclic ring; and
(b) removing the solvent to obtain a compound having the structural formula II:

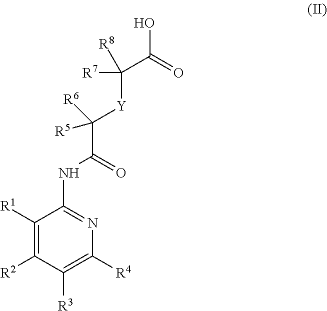

wherein R¹, R², R³, R⁴ R⁵, R⁶, R⁷, and R⁸ are as defined above.

In at least one embodiment of the substituted anhydride of formula III, the chemical group Y is a sulfur atom. In another embodiment of the anhydride of formula III, the chemical group Y is an oxygen atom. In another embodiment of the anhydride of formula III, the chemical group Y is an —SO— or an —SO₂— group. In a further embodiment of the anhydride of formula III, the chemical group Y is —CR⁹R¹⁰— wherein R⁹ and R¹⁰ are each independently chosen from hydrogen, C₁-C₄ linear or branched alkyl. In a still further embodiment when Y is R⁹ and R¹⁰ are each independently chosen from C₁-C₄ linear alkyl, R⁹ and R¹⁰ can be taken together to form a spirocyclic ring having from 4 to 7 atoms. In a still further embodiment when Y is sulfur, oxygen, —SO—, or —SO₂—, and R⁶ and R⁷ can be taken together to form a heterocyclic ring having from 4 to 6 carbon atoms. A range of specific anhydride compounds of formula III that can be used in the preparation of compound of formula II are further described in the Examples.

In at least one embodiment of the substituted 2-amino-pyridinyl compounds of formula IV, the chemical group at position R² is a hydrogen, and other non-hydrogen substituent groups are at positions R¹, R⁴, and optionally R³. Accordingly, exemplary compounds of structural formula IV include but are not limited to the compounds 4a, 4b, 4c, 4d, 4e, 4f, 4g, 4 h, 4i, 4j, 4k, 4l, 4m, 4n, 4o, 4p, 4q, 4r, 4s, 4t, 4u, 4v, 4w, 4x, 4y, 4z, 4aa, 4bb, 4cc, and 4dd, shown in Table 4 below.

TABLE 4

| | |
|---|---|
| 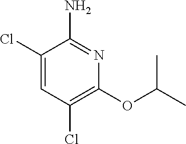 | 4a |
| 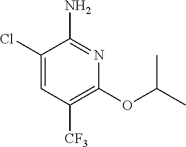 [SMILES: CC(C)Oc1nc(N)c(Cl)cc1C(F)(F)F] | 4b |
| 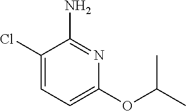 | 4c |
| 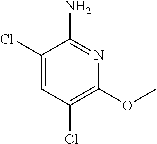 | 4d |
| 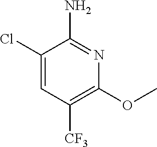 | 4e |
| 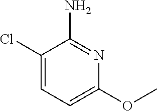 | 4f |
| 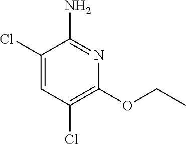 | 4g |
| 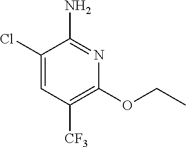 | 4h |
| 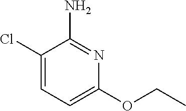 | 4i |
| 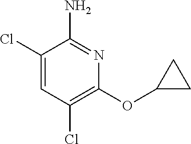 [SMILES: Nc1nc(OC2CC2)c(Cl)cc1Cl] | 4j |
| 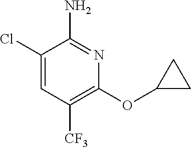 [SMILES: Nc1nc(OC2CC2)c(C(F)(F)F)cc1Cl] | 4k |
| 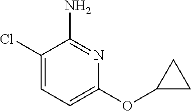 [SMILES: Nc1nc(OC2CC2)ccc1Cl] | 4l |
| 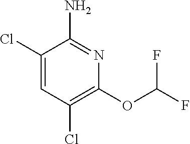 [SMILES: Nc1nc(OC(F)F)c(Cl)cc1Cl] | 4m |
| 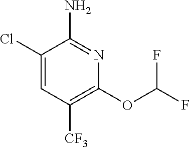 [SMILES: Nc1nc(OC(F)F)c(C(F)(F)F)cc1Cl] | 4n |
| 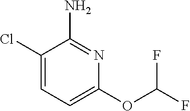 [SMILES: Nc1nc(OC(F)F)ccc1Cl] | 4o |
| 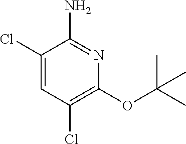 | 4p |
| 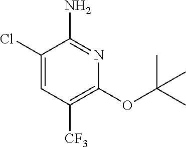 [SMILES: CC(C)(C)Oc1nc(N)c(Cl)cc1C(F)(F)F] | 4q |
| 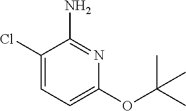 | 4r |
| 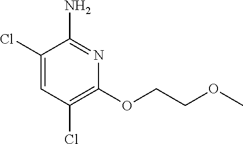 | 4s |
| 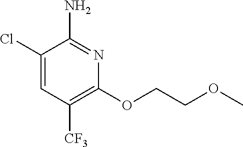 [SMILES: COCCOc1nc(N)c(Cl)cc1C(F)(F)F] | 4t |
| 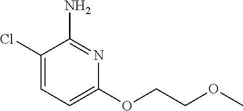 | 4u |
| 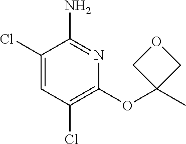 | 4v |
| 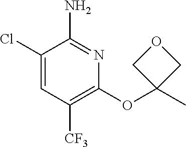 [SMILES: CC1(Oc2nc(N)c(Cl)cc2C(F)(F)F)COC1] | 4w |
| 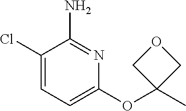 | 4x |
| 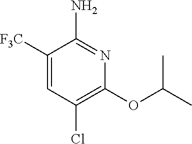 [SMILES: CC(C)Oc1nc(N)c(C(F)(F)F)cc1Cl] | 4y |
| 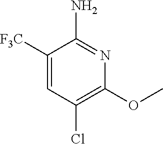 | 4z |
| 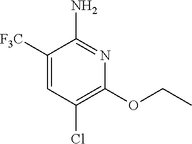 | 4aa |
| 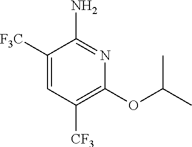 [SMILES: CC(C)Oc1nc(N)c(C(F)(F)F)cc1C(F)(F)F] | 4bb |
| 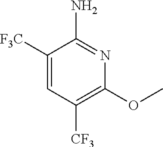 [SMILES: COc1nc(N)c(C(F)(F)F)cc1C(F)(F)F] | 4cc |
| 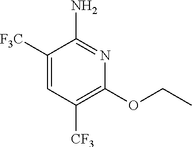 [SMILES: CCOc1nc(N)c(C(F)(F)F)cc1C(F)(F)F] | 4dd. |

Specific 2-amino-pyridinyl compounds of formula VI that can be used in the preparation of compound of formula II are further described in the Examples.

A non-limiting example of the general procedure for preparing the disclosed FABP3/4/5/7 inhibitors. A mixture of the substituted anhydride III and the substituted 2-amino-pyridinyl (1:0.75 molar ratio) is purged with argon then dissolved in dry dichloromethane. The reaction mixture is stirred for 24 h at room temperature. The solvent is removed using a rotary evaporator. The residue is then dissolved in 5 mL of ice-cold dichloromethane and transferred to a glass dram vial. The vial is then cooled on dry ice until visible crystals form. The resulting crystals are isolated via vacuum filtration and rinsed with ice cold dichloromethane. The crystals are allowed to air dry via vacuum filtration for 30 minutes. A small sample of isolated crystal is dissolved in acetone and purity is verified with silica thin-layer chromatography using a solvent system of 40% ethyl acetate in hexanes with 0.1% acetic acid. Plates are stained with PMA as a general stain and the formation of the carboxylic acid is verified with bromocresol green stain. Structure of the purified crystals is verified via ¹H NMR. The crystals are transferred to a clean, pre-weighed glass dram vial and yield is calculated. A range of specific synthesis procedures and reagents useful for preparing compounds of structural formula I and II, including certain specific compounds of Table 3 are provided in the Examples below.

Proposed Scheme B for Alternate Synthesis of R₅,R₆-substituted Analogs

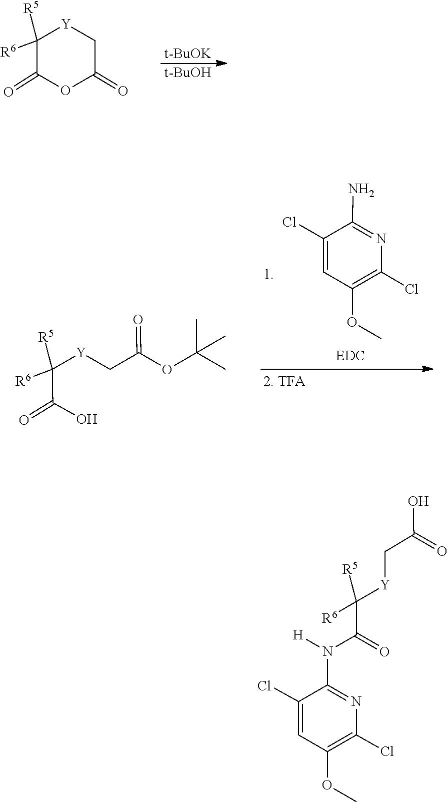

### Uses and Methods of Treatment

As noted elsewhere herein, FABP3/4/5/7 inhibitor compounds of structural formula I and II the present disclosure have been shown to provide potentially therapeutic effects, based on in vitro studies, pre-clinical, or clinical studies in a number of conditions, and diseases. Accordingly, the present disclosure contemplates that the inhibitor compounds of the present disclosure can be used in compositions and methods for treatment of diseases and/or conditions that are known to be affected by FABP3, FABP4, FABP5, and/or FABP7. Generally, methods for treating a subject having a disease or condition affected by FABP3, FABP4, FABP5, and/or FABP7 using a FABP3/4/5/7 inhibitor compound of the present disclosure comprises administering to the subject in need thereof, a therapeutically effective amount of a compound of structural formula I and II, or a pharmaceutical composition comprising such a compound and one or more pharmaceutically acceptable adjunct ingredients.

The conditions and diseases known to be affected by one or more of FABP4, FABP5, FABP3 and FABP7 which are contemplated for treatment using the FABP3/4/5/7 inhibitor compounds of structural formula I and II include, but are not limited to, the following: atherosclerosis, coronary atherosclerosis, arterial fibrosis, pulmonary hypertension, heart failure, obesity, Type-2 diabetes, Type-1-diabetes, gestational diabetes, polycystic ovary syndrome, endometriosis, conditions affected by lipid metabolism and free fatty acid serum levels, metabolic disorders, fatty liver disease, kidney fibrosis, systemic inflammation, acute inflammation, allergic inflammation, airway inflammation, viral infection (e.g., COVID-19, common cold), skin diseases (e.g., vitiligo, psoriasis, atopic dermatitis, allergic contact dermatitis, mycosis fungoides, alopecia areata, cicatricial alopecia, graft vs. host disease (GvHD), contact dermatitis, chronic eczema, dermatitis herpetiformis, cutaneous lupus, scleroderma, dermatomyositis, vasculitis, pemphigus, epidermolysis bullosa, linear IgA, and blistering disease), neurological conditions and diseases (e.g., pain, multiple sclerosis (MS), Parkinson's disease, experimental autoimmune encephalomyelitis (EAE), and ischemic stroke) and cancer (e.g., breast cancer, prostate cancer, ovarian cancer, skin cancer, gastric cancer, glioma, cholangiocarcinoma, bladder cancer, multiple myeloma, colorectal cancer, hepatocellular carcinoma, cervical cancer, oral squamous cell carcinoma, and/or non-small cell lung cancer (NSCLC)). Further specific description of various uses and treatment indications for the FABP3/4/5/7 inhibitor compounds of structural formula I and II are provided below.

### A. Cancer Treatment

As noted elsewhere herein, inhibition of FABP5 provides a method of inhibiting the metastasis of cancer cells in humans. Triple-Negative Breast Cancer (TNBC) accounts for about 10-20% of all breast cancers. The term “triple-negative breast cancer” refers to the fact that the cancer cells do not produce sufficient estrogen or progesterone receptors or make sufficient amounts of the protein Human Epidermal Growth Factor Receptor 2 (HEGR-2). Because tumors of TNBC lack definitive prognostic markers and selective targets for therapy, the treatment and management of this disease is a significant clinical problem and warrants an urgent need for a direct approach to inhibiting the biological processes which regulate development and metastasis of tumors. Without wishing to be limited by theory, data disclosed herein indicate that the disclosed FABP inhibitor compounds can provide inhibition of FABP5 and can thereby modulate the level of TNBC.

Accordingly, in at least one embodiment, the FABP3/4/5/7 inhibitor compounds of structural formula I and II can be used in methods treating cancer in a subject, wherein the methods comprise administering to a subject in need thereof a composition, comprising: (a) an effective amount of one or more of the disclosed FABP3/4/5/7 inhibitors or a pharmaceutically acceptable salt thereof; and (b) a pharmaceutically acceptable carrier or other adjunct ingredients. In at least one embodiment of the method, the subject can be diagnosed with, suffering from, and/or undergoing treatment for one or more cancers selected from breast cancer, prostate cancer, ovarian cancer, skin cancer, gastric cancer, glioma, cholangiocarcinoma, bladder cancer, multiple myeloma, colorectal cancer, hepatocellular carcinoma, cervical cancer, oral squamous cell carcinoma, and/or non-small cell lung cancer (NSCLC). In one embodiment of the disclosed cancer treatment method relates to breast cancer. In another embodiment of the disclose methods relate to preventing the metastasis of TNBC cells in a subject diagnosed with cancer.

Another still further aspect of the disclosed methods relates to methods treating cancer in a subject, comprising administering to a subject in need a composition, comprising: (a) an effective amount of one or more of the disclosed FABP3/4/5/7 inhibitors or a pharmaceutically acceptable salt thereof; and (b) a pharmaceutically acceptable carrier or other adjunct ingredients. In at least one embodiment of the method of treating cancer, the cancer is chosen from breast cancer, prostate cancer, ovarian cancer, hepatocellular carcinoma, multiple myeloma, neuroblastoma, lung adenocarcinoma or gastric carcinoma. In one example, the cancer is breast cancer. In a further example the cancer is prostate cancer. In another example the cancer is ovarian cancer. In a yet another example the cancer is hepatocellular carcinoma. In a still further example, the cancer is multiple myeloma. In another yet example the cancer is neuroblastoma. In a yet still further example, the cancer is lung adenocarcinoma. In a still yet another further example the cancer is gastric carcinoma.

In at least another embodiment, it is contemplated that the FABP3/4/5/7 inhibitors of the present disclosure can be used in a method of sensitizing cancer cells for treatment with other chemotherapeutic agents. Such agents can include standard chemotherapeutic compounds, such as doxorubicin, gemcitabine, cisplatin, paclitaxel, all-trans retinoic acid (atRA), a PARP inhibitor compound, and an immune checkpoint inhibitor compound, including but not limited to, an antibody that targets PD-1, or PD-L1. Accordingly, in at least one embodiment, the present disclosure provides a method of sensitizing cancer cells for treatment with other chemotherapeutic agents, wherein the method comprises contacting the cancer cells with one or more of the disclosed FABP3/4/5/7 inhibitors and contacting the cells with one or more chemotherapeutic agents. It is contemplated that this method can be carried out where the FABP3/4/5/7 inhibitor is contacted with the cancer cells prior to, or concurrently with, or after contacting the cells with the chemotherapeutic agent.

In at least one embodiment, the present disclosure also provides the use of a FABP3/4/5/7 inhibitor compounds of structural formula I or II, or a pharmaceutical composition comprising such a compound, for the manufacture of a medicament for treating a cancer in a subject. In at least one embodiment, the cancer treated by the use or medicament is chosen from breast cancer, prostate cancer, ovarian cancer, hepatocellular cancer, multiple myeloma, neuroblastoma, lung adenocarcinoma or gastric carcinoma.

### B. Fatty Acid Control

One aspect of the disclosed uses and methods relates to methods for inhibiting one or more of FABP3, FABP4, FABP5, and FABP7 in a subject, comprising administering to a subject in need a composition, comprising: (a) an effective amount of one or more of the FABP3/4/5/7 inhibitor compounds of structural formula I or II, or a pharmaceutically acceptable salt thereof; and (b) a pharmaceutically acceptable carrier or other adjunct ingredients.

A further aspect of the disclosed uses and methods relates to methods for controlling the free fatty acid serum levels in a subject, comprising administering to a subject in need a composition, comprising: (a) an effective amount of one or more of the disclosed FABP3/4/5/7 inhibitors of structural formula I or II, or a pharmaceutically acceptable salt thereof; and (b) a pharmaceutically acceptable carrier or other adjunct ingredients.

In at least one embodiment, the present disclosure also provides the use of a FABP3/4/5/7 inhibitor compound of the present disclosure, or a pharmaceutical composition comprising a FABP3/4/5/7 inhibitor compound of structural formula I or II, for the manufacture of a medicament for treating a disease or condition affected by FABP3/4/5/7 in a subject. In at least one embodiment, the disease or condition relates to control of the free fatty acid serum levels in a subject.

### C. Metabolic Disorders Treatment

Additionally, FABP4 and FABP5 are members of a family of small, soluble proteins which contribute to the trafficking of fatty acids within the cytosolic compartments of cells. These proteins have no catalytic function but transport hydrophobic fatty acids within the aqueous environment of the cytosol to the various destinations enabling fatty acid oxidation, membrane homeostasis or nuclear signaling. In addition, they are involved in signaling processes which are so far poorly understood. FABP4 is highly expressed in adipose tissue, macrophages, and endothelial cells. FABP5 is also expressed in macrophages, adipocytes, and endothelial cells, as well as in skin and several other tissues.

Without wishing to be limited by theory, in humans, plasma levels of FABP4 are increased in patients with metabolic syndrome and atherosclerosis. In addition, there is evidence for involvement of FABP4 in angiogenesis. More than a quarter of the population suffers from an aggregation of co-morbidities, including obesity, atherosclerosis, insulin resistance, dyslipidemias, coagulopathies, hypertension, and a pro-inflammatory state known as the metabolic syndrome. Patients with metabolic syndrome have high risk of atherosclerosis as well as Type 2 diabetes and other health problems. Like obesity, atherosclerosis has very limited therapeutic options.

A yet further aspect of the disclosed methods relates to methods for regulating insulin sensitivity in a subject, comprising administering to a subject in need a composition, comprising: (a) an effective amount of one or more of the FABP3/4/5/7 inhibitor compounds of structural formula I or II, or a pharmaceutically acceptable salt thereof; and (b) a pharmaceutically acceptable carrier or other adjunct ingredients.

A yet still further aspect of the disclosed methods relates to methods for treating Type-2 diabetes in a subject, comprising administering to a subject in need thereof a composition, comprising: (a) an effective amount of one or more of the FABP3/4/5/7 inhibitor compounds of structural formula I or II, or a pharmaceutically acceptable salt thereof; and (b) a pharmaceutically acceptable carrier or other adjunct ingredients.

A still yet further aspect of the disclosed methods relates to methods for the glucose plasma level in a subject, comprising administering to a subject in need thereof a composition, comprising: (a) an effective amount of one or more of the FABP3/4/5/7 inhibitor compounds of structural formula I or II, or a pharmaceutically acceptable salt thereof; and (b) a pharmaceutically acceptable carrier or other adjunct ingredients.

A yet still further aspect of the disclosed methods relates to methods for treating atherosclerosis in a subject, comprising administering to a subject in need thereof a composition, comprising: (a) an effective amount of one or more of the FABP3/4/5/7 inhibitor compounds of structural formula I or II, or a pharmaceutically acceptable salt thereof; and (b) a pharmaceutically acceptable carrier or other adjunct ingredients.

A yet further aspect of the disclosed methods relates to methods for treating liver steatosis in a subject, comprising administering to a subject in need thereof a composition, comprising: (a) an effective amount of one or more of the FABP3/4/5/7 inhibitor compounds of structural formula I or II, or a pharmaceutically acceptable salt thereof; and (b) a pharmaceutically acceptable carrier or other adjunct ingredients.

In at least one embodiment, the present disclosure also provides the use of a FABP3/4/5/7 inhibitor compound of structural formula I or II, or a pharmaceutical composition comprising a FABP3/4/5/7 inhibitor compound of the present disclosure, for the manufacture of a medicament for treating a metabolic disorder in a subject.

### D. Modulation of Immune Cell Activity and Populations

As described elsewhere herein, FABPs are involved in the regulation of immune cell activity. Without intending to be limited by mechanism, FABPs are believed to mediate immune cell metabolism, which is critical for the proper functioning of the immune system. More specifically, the activity of FABPs affects the utilization of fatty acids, thereby regulating energy production and the signaling pathways involved in the activation and function of immune cells. For example, FABP5 was found to regulate lipid metabolism and function in T-cells in the tumor microenvironment (TME) by mediating the uptake and oxidation of long-chain FAs in the cells. Further, tumor-infiltrating T lymphocytes (TIL) that express high FABP5 levels usually exhibit an exhausted phenotype and impaired anti-tumor activity due to the limited availability of glucose and high levels of long-chain FAs. Accordingly, inhibition of FABP5 in TILs is expected to activate the anti-tumor activity of the cell. Another Example is regulation of tissue resident T-cells (Trn) by FABP4/5. Trm cells are distinctive from other types of T-cells by having specialized function, location within tissues, gene expression signature, and metabolic needs. Trm cells rely on exogenous free fatty acids (FFA), that are internalized by FABP4/5 from the surrounding environment and metabolized within the cell to produce ATP needed for their maintenance and survival. T-cell-specific deficiency of FABP4/5 impaired uptake of FFA by Trm cells and significantly reduced their longevity and survival in vivo, while having no effect on survival of central memory T (TCM) cells in lymph nodes. Hence, FABP4/5 inhibitors can be used to specifically target Ta cells in autoimmune diseases and other disorders. FABP4 is highly expressed in Ly6C⁻MHCII⁻CD36⁺ circulating monocyte/macrophages to facilitate oxidative lipid uptake, foam cell formation, angiogenesis, tissue remodeling and pro-tumor functions. This high expression suggests that inhibition of FABP4 can enhance ani-tumor immune responses in cancer cells and can also block formation of foam cells and chronic inflammation in obesity. Overall, the role of FABPs in immune cell regulation highlights the importance of lipid metabolism in the regulation of the immune system and imply that targeting FABPs may be a promising strategy for improving immune cell function and treating a wide range of diseases and conditions caused by chronic inflammation and cancer.

A yet still further aspect of the disclosed methods relates to methods for treating autoimmune diseases that are mediated by tissue resident T-cells (Trm) (e.g., experimental autoimmune encephalomyelitis (EAE), asthma, type-1-diabetes, autoimmune lung disease, autoimmune hepatitis, rheumatoid arthritis (RA), spondyloarthropathy, vesicular stomatitis virus infection, multiple sclerosis (MS), lupus nephritis, Crohn's disease, ulcerative colitis, food allergy, and graft versus host disease (GvHD)) in a subject, comprising administering to a subject in need thereof a composition, comprising: (a) an effective amount of one or more of the FABP3/4/5/7 inhibitor compounds of structural formula I or II, or a pharmaceutically acceptable salt thereof; and (b) a pharmaceutically acceptable carrier or other adjunct ingredients.

A method for modulating immune cell populations and/or immune cell activity in a subject in need thereof, comprising administering to a subject in need thereof a therapeutically effective amount of a compound of structural formula I or II, or a pharmaceutical composition of such a compound. In at least one embodiment, the subject in need thereof has a disease or disorder caused by, affected by, and/or characterized by immune cell populations and/or immune cell activity, for example, wherein the immune cells are M2 macrophages, tumor associated macrophages (TAMs), regulatory T-cells (Tregs), or tissue resident T-cells (Trm). In at least one embodiment, the subject in need of a treatment for modulating immune cell populations and/or immune cell activity has been diagnosed with, is suffering from, or is being treated for cancer. In another embodiment, it is contemplated that the subject in need of a treatment for modulating immune cell populations and/or immune cell activity has an autoimmune disease or disorder.

### Pharmaceutical Compositions

The present disclosure also provides uses and methods in which a FABP4/5 inhibitor compounds, such as a compound of structural formula I, is administered to a subject in the form of a pharmaceutical composition. In such embodiments, the pharmaceutical composition includes a therapeutically effective amount of the FABP3/4/5/7 inhibitor compound (e.g., compound of Tables 1-8), or a pharmaceutically acceptable salt or ester of such a compound and one or more pharmaceutically acceptable carriers. Such pharmaceutical compositions can be prepared using methods well known in the pharmaceutical art (see, e.g., Remington's Pharmaceutical Sciences, Mace Publishing Co., Philadelphia, PA 17th Ed. (1985) and Modern Pharmaceutics, Marcel Dekker, Inc. 3rd Ed. (G.S. Banker & C.T. Rhodes, Eds.). Methods of preparing pharmaceutical compositions of FABP3/4/5/7 inhibitor compounds are described in the present disclosure, including the Examples disclosed herein.

In at least one embodiment, the present disclosure provides a pharmaceutical composition comprising: an effective amount of one or more of the disclosed FABP3/4/5/7 inhibitors, such as a compound of structural formula I; and one or more adjunct ingredients, such as a pharmaceutically acceptable carrier. The disclosed compositions can comprise from about 10% to about 95% by weight of one or more of the disclosed FABP3/4/5/7 inhibitors. In another embodiment, the compositions comprise from about 10% to about 80% by weight of one or more of the disclosed FABP3/4/5/7 inhibitors. In a further embodiment the compositions comprise from about 20% to about 50% by weight of one or more of the disclosed FABP3/4/5/7 inhibitors. In a still further embodiment, the compositions comprise from about 50% to about 90% by weight of one or more of the disclosed FABP3/4/5/7 inhibitors. In a yet another embodiment the compositions comprise from about 70% to about 90% by weight of one or more of the disclosed FABP3/4/5/7 inhibitors. In a yet further embodiment, the compositions comprise from about 80% to about 95% by weight of one or more of the disclosed FABP3/4/5/7 inhibitors. In a still yet further embodiment, the compositions comprise from about 90% to about 95% by weight of one or more of the disclosed FABP3/4/5/7 inhibitors.

Generally, the pharmaceutical compositions can be prepared by diluting the active ingredient(s) with an excipient and/or enclosing it within a carrier in the form of a capsule, sachet, paper or other container. When the excipient serves as a diluent, it can be a solid, semi-solid, or liquid material (as above), which acts as a vehicle, carrier or medium for the active ingredient. Thus, the pharmaceutical composition(s) suitable for administering in the methods of the disclosure can be in the dosage form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

The carriers used in the preparation of the pharmaceutical compositions can include excipients such as inert solid diluents and fillers, diluents, including sterile aqueous solution and various organic solvents, permeation enhancers, solubilizers and adjuvants. Suitable excipients for use in the pharmaceutical compositions comprising a celastrol derivative of the present disclosure are well known in the art and include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The pharmaceutical compositions can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

In the uses and methods of treatment, it is contemplated that the pharmaceutical composition comprising the FABP3/4/5/7 inhibitor compounds, such as a compound of structural formula I, can be administered either as single or multiple doses, and by any of the accepted modes of administration of active ingredients having similar utility. For example, a pharmaceutical composition comprising an celastrol derivative can be administered using a variety of different modes including oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

The pharmaceutical compositions including the FABP3/4/5/7 inhibitor compounds of the present disclosure can be used in a range of therapeutic methods of treatment and a range of dosages are contemplated for administration of a pharmaceutically effective amount. The dosage and frequency (single or multiple doses) of administration of the pharmaceutical composition to a subject can vary depending upon a range of factors, such as, the route of administration; the subject's size, age, sex, health, body mass, and/or diet; the state of the disease being treated; whether the subject is suffering from any other diseases, and any concurrent treatment being received. One of ordinary skill will understand that adjustment of established dosages (e.g., frequency and duration) to obtain the therapeutically effective amount may be required depending on the subject. Typically, the amount of a pharmaceutical composition containing a FABP3/4/5/7 inhibitor compound to be administered to a subject in a therapeutic method of treatment will be determined by a physician, in view of relevant circumstances of the subject being so treated, the chosen route of administration, and of course, the age, the weight, the severity of symptoms, the response of the individual subject to the treatment, and the like.

Generally, a therapeutically effective amount is the amount sufficient for the administered composition to accomplish a desired therapeutic purpose relative to the absence of the compound. For example, the therapeutically effective amount can be the amount determined to be sufficient to contribute to the treatment, prevention, or reduction of a symptom or symptoms of a disease. Methods for determining the dosage providing a therapeutically effective amount of a compound are well-known to those of ordinary skill in the art, and typically are based on analysis of amounts determined in cellular assays and/or animal models. For example, a dosage for administration to humans can be formulated to achieve a concentration that has been observed as therapeutically effective in an animal model. The dosage in the pharmaceutical composition for humans can further be adjusted by monitoring the effectiveness and adjusting upwards or downwards. One of ordinary skill can used methods well known in the art to adjust the dosage in a pharmaceutical composition of the present disclosure to achieve maximal therapeutic efficacy for humans.

Generally, methods for therapeutic treatment are developed by starting with a pharmaceutical composition containing less than the optimal dose of the FABP3/4/5/7 inhibitor compound. Thereafter, the dosage of the compound is increased incrementally until optimal efficacy is attained. A key factor considered in developing the optimal dose is the ratio between the toxicity and the therapeutic efficacy of the active ingredient. This ratio, referred to as the compound's therapeutic index, is typically described as the ratio of the active ingredient's LD₅₀ (the amount of compound lethal in 50% of the population) to its ED₅₀ (the amount of compound effective in 50% of the population). Typically, a higher therapeutic index for a compound is preferred. Therapeutic index data can be obtained from cell culture assays and/or animal model studies and then used to determine a safe range of dosages of the active ingredient in a pharmaceutical composition for administration to humans. Ideally the dosage determined provides the active ingredient at its ED₅₀ level in the subject with little or no toxicity.

Solid form preparations of pharmaceutical compositions can include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, generally the carrier is a finely divided solid that is in an admixture with the finely divided active component, e.g., a disclosed FABP3/4/5/7 inhibitor. In tablets, the active ingredient is mixed with the carrier having the necessary binding properties in suitable proportions and compacted in the shape and size desired. The solid form preparation of a pharmaceutical composition of the present disclosure can comprise from about 0.5% to about 10% by weight of a binding agent. Non-limiting examples of binding agents suitable for use in the disclosed compositions are chosen from polyethylene glycol 1500, polyethylene glycol 2000, polyethylene glycol 3000, polyethylene glycol 4000, polyethylene glycol 6000, polyethylene glycol 8000, polyethylene glycol 10000, methylcellulose, ethylcellulose, hydroxymethyl cellulose, hydroxyethylcellulose, hydroxypropyl methylcellulose, polyoxyethylene, copolymers of polyoxyethylene-polyoxypropylene and mixtures thereof. In one embodiment the binding agent is methylcellulose, ethylcellulose, hydroxymethyl cellulose, or hydroxyethylcellulose. In one non-limiting example the binding agent is ethylcellulose.

In some embodiments, the solid compositions can comprise from about 0.5% to about 10% by weight of a carrier. Non-limiting examples of solid carriers include: starch such as tapioca starch, corn starch, potato starch, gelatin, dextrin, inulin, cyclodextrin, oxidized starch, starch ester, starch ether, crosslinked starch, alpha starch, octenyl-succinate ester, and processed starch obtained by treating a starch by an acid, heat, or enzyme, or an emulsifier such as gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, and combinations thereof, or a sugar alcohol that can optionally have humectant properties such as ethylene glycol, glycerol, erythritol, threitol, arabitol, xylitol, ribitol, mannitol, sorbitol, galactitol, frucitol, iditol, sucrose, fructose, isomalt, maltitol, lactitol, sorbitol, dextrose or inositol, and combinations thereof.

The disclosed compositions can comprise from about 25 mg to about 1200 mg of one or more of the disclosed FABP3/4/5/7 inhibitor. In one aspect the disclosed single dose compositions of a disclosed FABP3/4/5/7 inhibitor can comprise any amount from about 25 mg to about 500 mg.

In a further aspect the disclosed single dose compositions of a disclosed FABP3/4/5/7 inhibitor can comprise any amount from about 100 mg to about 500 mg. In a yet further aspect, the disclosed single dose compositions of a disclosed FABP3/4/5/7 inhibitor can comprise any amount from about 500 mg to about 1000 mg.

The single dose compositions can comprise any amount of FABP3/4/5/7 inhibitor from about 25 mg to about 250 mg. For example, the disclosed compositions can comprise 25 mg, 26 mg, 27 mg, 28 mg, 29 mg, 30 mg 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 90 mg, 91 mg, 92 mg, 93 mg, 94 mg, 95 mg, 96 mg, 97 mg, 98 mg, 99 mg, 100 mg, 101 mg, 102, mg, 103, mg, 104 mg, 105 mg, 106 mg, 107 mg, 108 mg, 109 mg, 120 mg, 121 mg, 122 mg, 123 mg, 124 mg, 125 mg, 126 mg, 127 mg, 128 mg, 129 mg, 120 mg, 121 mg, 122 mg, 123 mg, 124 mg, 125 mg, 126 mg, 127 mg, 128 mg, 129 mg, 130 mg 31 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 190 mg, 191 mg, 192 mg, 193 mg, 194 mg, 195 mg, 196 mg, 197 mg, 198 mg, 199 mg, 200 mg, 201 mg, 202, mg, 203, mg, 204 mg, 205 mg, 206 mg, 207 mg, 208 mg, 209 mg, 210 mg, 212 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg of one or more of the disclosed FABP3/4/5/7 inhibitors.

Liquid forms of the pharmaceutical compositions can include, for example, solutions suitable for oral or parenteral administration, suspensions, and emulsions suitable for oral administration. Sterile water solutions of the active component or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like. In at least one embodiment, the disclosed liquid compositions can comprise from about 5% to about 25% by weight of a liquid carrier.

For liquid embodiments of the present compositions, the targeted cells, for example, cancer cells or tumor cells can be contacted with an aqueous solution comprising from about 0.5 μg/mL to about 250 μg/mL. In one embodiment the compositions can comprise from about 1 μg/mL to about 100 μg/mL. In another embodiment the compositions can comprise from about 10 μg/mL to about 100 μg/mL. In a further embodiment the compositions can comprise from about 5 μg/mL to about 20 μg/mL. In a yet further embodiment, the compositions can comprise from about 1 μg/mL to about 50 μg/mL. In a yet another embodiment the compositions can comprise from about 1 μg/mL to about 10 μg/mL. In a still further embodiment, the compositions can comprise from about 15 μg/mL to about 50 μg/mL. In still another embodiment the compositions can comprise from about 20 μg/mL to about 200 μg/mL.

The disclosed compositions can provide a single dose of a disclosed FABP3/4/5/7 inhibitor based upon the body mass of the subject being treated. Therefore, a single dose of a disclosed FABP3/4/5/7 inhibitor can range from about 0.35 mg/kg to about 20 mg/kg of the subject's body mass. In one embodiment, the amount of a disclosed FABP3/4/5/7 inhibitor in a single dose is from about 1 mg/kg to about 8 mg/kg of the subject's body mass. In another embodiment, the amount of a disclosed FABP3/4/5/7 inhibitor in a single dose is from about 2 mg/kg to about 5 mg/kg of the subject's body mass. In a further embodiment, the amount of a disclosed FABP3/4/5/7 inhibitor in a single dose is from about 1.5 mg/kg to about 4 mg/kg of the subject's body mass. In a yet further embodiment, the amount of a disclosed FABP3/4/5/7 inhibitor in a single dose is from about 4 mg/kg to about 10 mg/kg of the subject's body mass. In a still further embodiment, the amount of a disclosed FABP3/4/5/7 inhibitor in a single dose is from about 5 mg/kg to about 8 mg/kg of the subject's body mass.

For example, the dose can comprise any amount from about 0.5 mg/kg to about 10 mg/kg on the body mass of the subject being treated. For example, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, or 50 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 90 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, or 10.0 mg/kg of body mass.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Preparation of 2-((2-((3-chloro-6-isopropoxy-5-(trifluoromethyl)pyridin-2-yl)amino)-2-oxoethyl)thio)acetic acid (PD151: Compound 1)

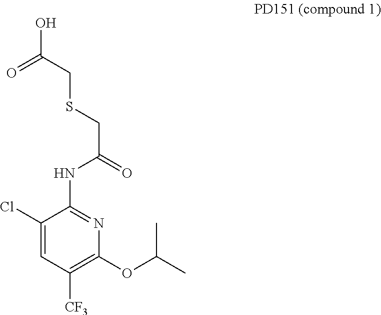 [SMILES: CC(C)Oc1nc(NC(=O)CSCC(=O)O)c(Cl)cc1C(F)(F)F]

This example illustrates the preparation of the FABP 4/5 inhibitor compound PD151 via the synthetic method of Schemes 1-5 as shown below.

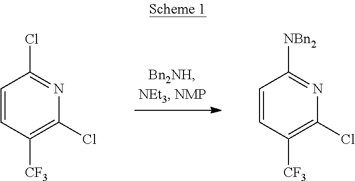

### Materials and Methods

To a stirred solution of 2,6-dichloro-3-(trifluoromethyl)pyridine (5 g, 23.15 mmol in N-Methyl-2-pyrrolidinone (50 mL) was added trimethylamine (2.343 g, 23.15 mmol), and dibenzylamine (4.57 g, 23.15 mmol) at RT. The reaction mixture was heated to 120° C. and stirred for 16 h. Progress of reaction was monitored by TLC (5% EtOAc in pet. ether). On completion of the reaction, the reaction mixture was quenched in ice cold water (300 mL) and extracted with Ethyl acetate (2×250 mL). The combined organic layers were washed with brine, dried over sodium sulphate and concentrated over rotary evaporator under reduced pressure (bath temperature 45° C.) to get crude compound. The crude product mixture was purified by column chromatography (Isolera) by using ethyl acetate in hexane as an eluting solvent system (product eluted at 8% EtOAc in Hexane) to yield pure N,N-dibenzyl-6-chloro-5-(trifluoromethyl)pyridin-2-amine (7.5 g, 23.15 mmol, 83% yield) as a colorless liquid.

NMR and LCMS analysis confirmed preparation of the desired intermediate compound. ¹H NMR (400 MHz, DMSO-d₆) δ: 3.33 (s, 4H), 6.65 (d, J=8.0 Hz, 1H), 7.36-7.27 (m, 10H), 7.83 (d, J=8.0 Hz, 1H). LCMS Rt=3.48 min, [M+H] found 377.0.

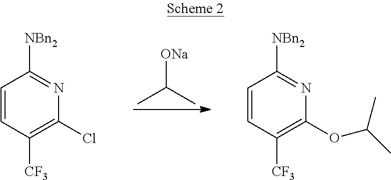

### Materials and Methods

To a stirred solution of N,N-dibenzyl-6-chloro-5-(trifluoromethyl)pyridin-2-amine (500 mg, 1.327 mmol) in 2-propanol (10 ml) was added sodium propan-2-olate (1.327 mL, 1.327 mmol) at RT. The reaction mixture was stirred at 80° C. for 16h. Progress of reaction was monitored by TLC (5% EtOAc in pet. ether). On completion of the reaction, the reaction mixture was quenched in ice cold water (100 mL) and extracted with ethyl acetate (2×100 mL). Combined organic layers were washed with brine, dried over sodium sulphate and concentrated over rotary evaporator under reduced pressure (bath temperature 45° C.) to yield crude N,N-dibenzyl-6-isopropoxy-5-(trifluoromethyl)pyridin-2-amine (0.6 g, 1.327 mmol, 63.2% yield), which was used in the next reaction without further purification.

LCMS confirmed formation of the desired intermediate compound. LCMS Rt=3.15 min, [M+H] found 401.2.

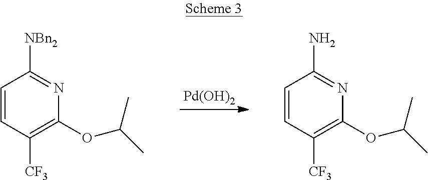

### Materials and Methods

To a stirred solution of N,N-dibenzyl-6-isopropoxy-5-(trifluoromethyl)pyridin-2-amine (3 g, 7.49 mmol) in MeOH (45 mL) and AcOH (5 mL) was added Pd(OH)₂ (0.500 g, 3.56 mmol) at RT and stirred under 1 bar hydrogen pressure for 16h. Reaction progress was monitored by TLC (5% EtOAc in pet ether). On completion of the reaction, the reaction mixture was filtered through celite and washed with ethyl acetate. The filtrate MLs were concentrated over rotary evaporator under reduced pressure (bath temperature 45° C.) to get crude product. Crude mixture was purified by column chromatography (Isolera) by using ethyl acetate in hexane as an eluting solvent system (product eluted at 5% EtOAc in hexane) to obtain 6-isopropoxy-5-(trifluoromethyl)pyridin-2-amine (0.81 g, 3.6 mmol, 48.1% yield) as a colorless liquid.

NMR and LCMS analysis confirmed preparation of the desired intermediate compound. ¹H NMR (400 MHz, DMSO-d₆) δ:1.26 (d, J=6.0 Hz, 6H), 5.31-5.25 (m, 1H), 6.01 (d, J=8.40 Hz, 1H), 6.59 (s, 2H), 7.50 (d, J=8.40 Hz, 1H). LCMS Rt=2.76 min, [M+H] found 221.0.

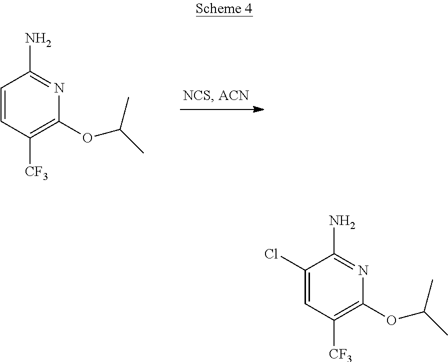

### Materials and Methods

To a stirred solution 6-isopropoxy-5-(trifluoromethyl)pyridin-2-amine (700 mg, 3.18 mmol) in acetonitrile (70 mL) was added N-chlorosuccinimide (679 mg, 5.09 mmol) at RT. The solution was stirred at 100° C. for 6 h. The reaction was monitored by TLC (5% EtOAc in pet. ether). On completion of the reaction, the reaction mixture was quenched in ice cold water (100 mL) and extracted with ethyl acetate (3×100 mL). Combined organic layers were washed with brine solution, dried over sodium sulphate, and concentrated over rotary evaporator under reduced pressure (bath temperature 45° C.) to get crude product. The crude mixture was purified by column chromatography (Isolera) using ethyl acetate in hexane as an eluting solvent system (product eluted at 2% EtOAc in hexane) to yield 3-chloro-6-isopropoxy-5-(trifluoromethyl)pyridin-2-amine (0.350 g, 1.33 mmol, 41.9% yield) as a pale yellow liquid.

NMR and LCMS analyses confirmed preparation of the desired intermediate compound. ¹H NMR (400 MHz, DMSO-d₆) δ:1.27 (d, J=6.0 Hz, 6H), 5.30-5.21 (m, 1H), 6.97 (s, 2H), 7.69 (s, 1H). LCMS Rt=2.32 min, [M+H] found 255.0.

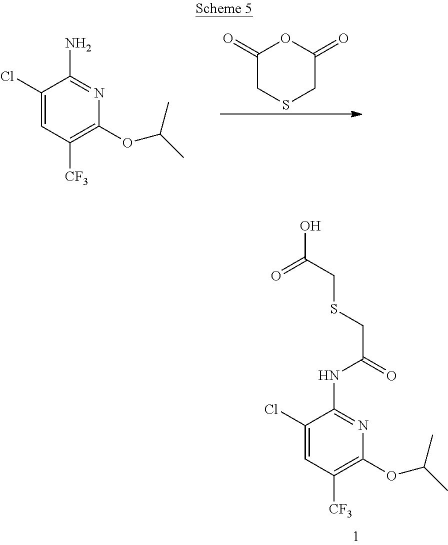 [SMILES: CC(C)Oc1nc(N)c(Cl)cc1C(F)(F)F ; O=C1CSCC(=O)O1 ; CC(C)Oc1nc(NC(=O)CSCC(=O)O)c(Cl)cc1C(F)(F)F]

### Materials and Methods

To a stirred solution of 3-chloro-6-isopropoxy-5-(trifluoromethyl)pyridin-2-amine (350 mg, 1.375 mmol) in dioxane (5 mL) was added 1,4-oxathiane-2,6-dione (218 mg, 1.649 mmol) at RT. The reaction mixture was stirred at 110° C. for 16 h. The reaction was monitored by TLC (40% EtOAc in pet. ether). On completion of the reaction, the reaction mixture was quenched in ice cold water (50 mL) and extracted with ethyl acetate (3×50 mL). Combined organic layers were washed with brine, dried over sodium sulphate, and concentrated over rotary evaporator under reduced pressure (bath temperature 45° C.) to get crude product mixture. Crude product was purified by PREP-HPLC using ammonium bicarbonate buffer and the pure fractions were lyophilized to obtain 2-((2-((3-chloro-6-isopropoxy-5-(trifluoromethyl)pyridin-2-yl)amino)-2-oxoethyl)thio)acetic acid (PD151; compound 1) (0.074 g, 0.199 mmol, 13.89% yield) as an off white solid.

NMR and LCMS analyses confirmed preparation of the desired product compound. ¹H NMR (400 MHz, DMSO-d₆) δ: 1.32 (d, J=6.00 Hz, 6H), 3.42 (s, 2H), 3.59 (s, 2H), 5.31-5.22 (m, 1H), 8.21 (s, 1H), 10.50 (s, 1H), 12.67 (s, 1H). LCMS Rt=2.03 min, [M+H] found 387.0.

### Example 2: Preparation of 2-((2-((3,5-dichloro-6-isopropoxypyridin-2-yl)amino)-2-oxoethyl)thio)acetic acid (PD152: Compound 2)

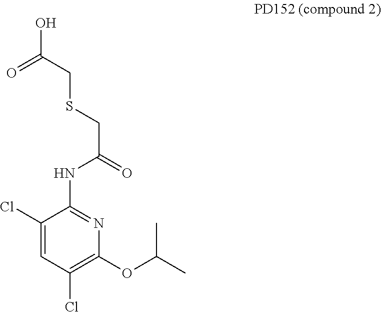 [SMILES: CC(C)Oc1nc(NC(=O)CSCC(=O)O)c(Cl)cc1Cl]

This example illustrates the preparation of the FABP 4/5 inhibitor compound 2 (PD152) via the synthetic method of Schemes 6-7 as shown below.

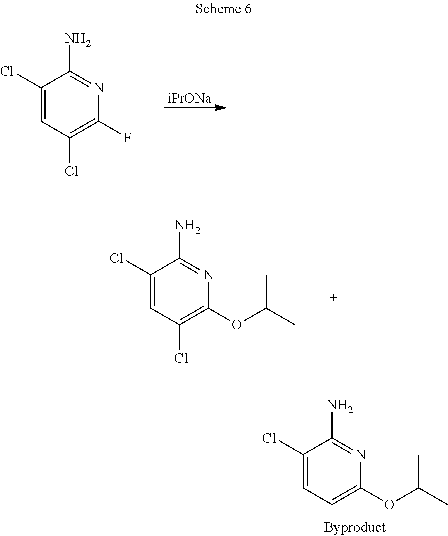

### Materials and Methods

To a solution of 3,5-dichloro-6-fluoropyridin-2-amine (500 mg, 2.76 mmol) in isopropanol (2.0 ml) was added 1 M sodium isopropoxide in THF (10.0 ml). The resulting mixture was stirred at 100° C. for 16h. Progress of reaction was monitored by TLC (20% EtOAc in pet ether) and LCMS. After completion of reaction, reaction mixture was quenched with water (20 mL). Reaction mixture was extracted with ethyl acetate (3×30 mL). Combined organic layers were washed with brine, dried over sodium sulphate, and concentrated over rotary evaporator under reduced pressure to get a crude product mixture. The crude mixture was purified by column chromatography (Isolera) by using EtOAc and pet ether as an eluting solvent system (product eluted at 15-20% EtOAc in pet. ether) to yield a mixture of 3,5-dichloro-6-isopropoxypyridin-2-amine and mono des-chloro byproduct (400 mg, 1.040 mmol, 37.6% yield) as a color less liquid.

NMR analysis confirmed preparation of the desired intermediate compound mixture. ¹H NMR (400 MHz, DMSO-d₆) δ: 1.28 (d, J=6.00 Hz, 6H), 5.22-5.15 (m, 1H), 6.35 (br s, 1H), 7.64 (s, 1H)

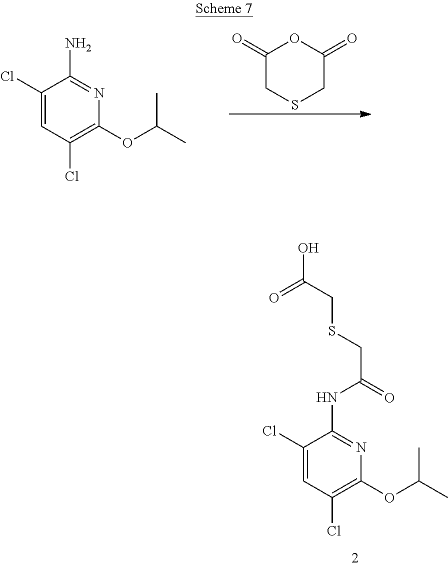 [SMILES: CC(C)Oc1nc(N)c(Cl)cc1Cl ; O=C1CSCC(=O)O1 ; CC(C)Oc1nc(NC(=O)CSCC(=O)O)c(Cl)cc1Cl]

### Materials and Methods

To a reaction vial containing a solution of crude 3,5-dichloro-6-isopropoxypyridin-2-amine (350 mg, 1.583 mmol) in dioxane (5.0 mL) was added 1,4-oxathiane-2,6-dione (314 mg, 2.375 mmol). The resulting mixture was stirred at 120° C. for 48h. Progress of reaction was monitored by TLC (50% (10 mL) EA in pet ether+0.5 ml acetic acid) and LCMS. Cooled to RT, solvents were removed by rotary evaporation to yield product compound as crude liquid. Crude product was passed through column (Isolera) by using MeOH and DCM as an eluting solvent system (product eluted at 2-3% MeOH in DCM) to get mixture of products, which were separated through prep-HPLC. Obtained product was extracted by ethyl acetate and washed with sat NaHCO3 solution. The organic layer was dried over sodium sulphate and solvent removed under vacuum to yield 2-((2-((3,5-dichloro-6-isopropoxypyridin-2-yl)amino)-2-oxoethyl)thio)acetic acid (PD152; compound 2) ((92.2 mg, 0.260 mmol, 16.41% yield) as a white solid.

NMR and LCMS analyses confirmed preparation of the desired product compound. ¹H NMR (400 MHz, DMSO-d₆) δ: 1.32 (d, J=6.00 Hz, 6H), 3.43 (s, 2H), 3.51 (s, 2H), 5.21-5.15 (m, 1H), 8.15 (s, 1H), 10.30 (s, 1H), 12.62 (br s, 1H). LCMS Rₜ=1.89 min, [M+H] found 353.0.

### Example 3: Preparation of 2-((2-((3-chloro-6-isopropoxypyridin-2-yl)amino)-2-oxoethyl)thio)acetic acid (PD152.2: Compound 3)

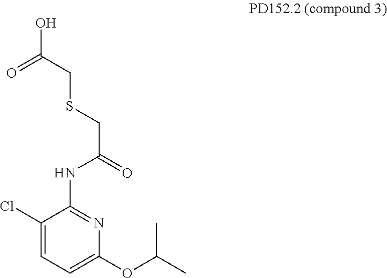 [SMILES: CC(C)Oc1ccc(Cl)c(NC(=O)CSCC(=O)O)n1]

This example illustrates the preparation of the FABP 4/5 inhibitor compound PD152.2.

### Materials and Methods

2-((2-((3-chloro-6-isopropoxypyridin-2-yl)amino)-2-oxoethyl)thio)acetic acid, compound 3 (PD152.2) is obtained as a by-product of the reactions described in Example 2, Schemes 6-7.

NMR and LCMS analyses confirmed preparation of the desired product compound. ¹H NMR (400 MHz, DMSO-d₆) δ: 1.28 (d, J=6.00 Hz, 6H), 3.43 (s, 2H), 3.51 (s, 2H), 5.15-5.08 (m, 1H), 6.67 (d, J=8.50 Hz, 1H), 7.80 (d, J=8.50 Hz, 1H), 10.21 (s, 1H), 12.65 (br s, 1H). LCMS Rₜ=1.67 min, [M+H] found 319.0.

### Example 4: Preparation of 2-((2-((3,5-dichloro-6-isopropoxypyridin-2-yl)amino)-2-oxoethyl)thio)-2-methylpropanoic acid (PD153; Compound 4)

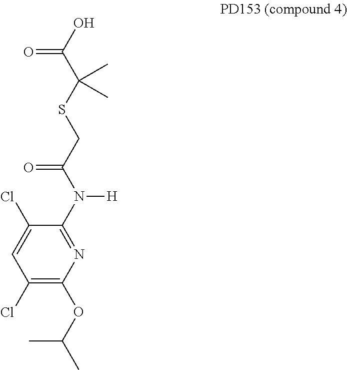 [SMILES: CC(C)Oc1nc(NC(=O)CSC(C)(C)C(=O)O)c(Cl)cc1Cl]

This example illustrates the preparation of the FABP 4/5 inhibitor compound PD153 via the synthetic method of Schemes 8-11 as shown below.

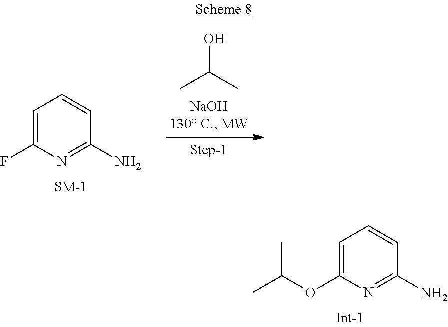

### Materials and Methods

To a stirred solution of 6-fluoropyridin-2-amine SM-1 (500 mg, 4.46 mmol, 1.0 eq) in 2-propanol (4 mL), sodium hydroxide (440 mg, 10.89 mmol, 2.45 eq) was added and the resultant reaction mixture stirred at 150° C. for 5 minutes in microwave. Progress of the reaction was monitored by TLC. After complete completion of the starting materials, the reaction mixture was diluted with ice cold water (30 mL) and extracted with ethyl acetate (2×30 mL). The combined organic extracts were washed with water (20 mL) and then brine solution (20 mL) and dried over anhydrous sodium sulphate, filtered, and concentrated under reduced pressure to obtain crude compound, which was purified by combi-flash chromatography, eluting with 25% ethyl acetate in heptane to afford 6-isopropoxypyridin-2-amine Int-1 (600 mg, 84%) as colorless liquid.

LCMS analysis confirmed preparation of the desired Int-1 compound. 95.04%; ESI MS m/z calcd. For C₈H₁₂N₂O ([M+H]⁺) 153.09; found 153.20;

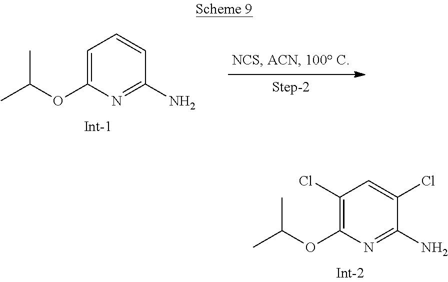

### Materials and Methods

To a stirred solution of 6-isopropoxypyridin-2-amine, Int-1 (250.00 mg, 1.64 mmol, 1.0 eq) in acetonitrile (4 mL), N-chlorosuccinimide (447 mg, 3.28 mmol, 2.0 eq) was added and the resultant reaction mixture stirred at 100° C. for 60 min in microwave. Progress of the reaction was monitored by TLC. After complete completion of the starting materials, the reaction mixture was diluted with ice cold water (30 mL) and extracted with ethyl acetate (2×30 mL). The combined organic extracts were washed with water (30 mL) and then brine solution (30 mL) and dried over anhydrous sodium sulphate, filtered, and concentrated under reduced pressure to obtain crude compound, which was purified by combi-flash chromatography, eluting with 20% ethyl acetate in heptane to afford 3,5-dichloro-6-isopropoxy-pyridin-2-amine, Int-2 (200 mg, 52%) as a yellow colored liquid

NMR and LCMS analyses confirmed preparation of the desired Int-2 compound. ¹H NMR (400 MHz, CDCl₃) δ 7.42 (s, 1H) 5.23-5.17 (m, 1H) 4.64 (br s, 2H) 1.34-1.33 (d, 6H). LCMS: 92.78%; ESI MS m/z calcd. For C₈H₁₀Cl₂N₂O ([M+H]+) 221.08; found 221.0;

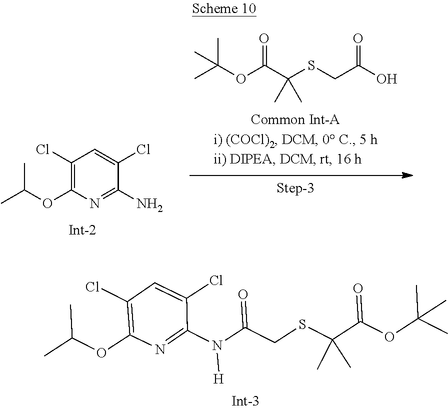 [SMILES: CC(C)(C)OC(=O)C(C)(C)SCC(=O)O ; CC(C)Oc1nc(N)c(Cl)cc1Cl ; CC(C)Oc1nc(NC(=O)CSC(C)(C)C(=O)OC(C)(C)C)c(Cl)cc1Cl]

### Materials and Methods

To a stirred solution of 2-(2-tert-butoxy-1,1-dimethyl-2-oxo-ethyl)sulfanylacetic acid Common Int-A(200 mg, 0.79 mmol, 1.0 eq) in DCM (2 mL), oxalyl chloride (200 mg, 1.58 mmol, 2.0 eq) was added at 0° C. and then DMF(cat) was added and the resultant reaction mixture stirred for 5 h at room temperature. Progress of reaction was monitored by TLC and LCMS. After complete conversion of starting material, the reaction mixture was concentrated under reduced pressure to get crude compound of tert-butyl 2-(2-chloro-2-oxo-ethyl)sulfanyl-2-methyl-propanoate (200 mg, crude) as a brown liquid.

To a stirred solution of 3,5-dichloro-6-isopropoxy-pyridin-2-amine Int-2 (200 mg, 0.90 mmol, 1.0 eq) in DCM (5 mL), N,N-Diisopropylethylamine (550 mg, 4.5 mmol, 5.0 eq) and tert-butyl 2-(2-chloro-2-oxo-ethyl)sulfanyl-2-methyl-propanoate (200 mg) were added at 0° C. The resulting reaction mixture was stirred for 16 h at room temperature. Progress of reaction was monitored by TLC. After complete conversion of starting material, the reaction mixture was diluted with ice cold water (30 mL) and extracted with ethyl acetate (2×30 mL). The combined organic extracts were washed with water (30 mL) and then brine solution (30 mL) and dried over anhydrous sodium sulphate, filtered, and concentrated under reduced pressure to obtain crude compound of tert-butyl 2-[2-[(3,5-dichloro-6-isopropoxy-2-pyridyl)amino]-2-oxo-ethyl]sulfanyl-2-methyl-propanoate Int-3 (200 mg, crude) as a brown liquid. The crude was taken for the next step.

LCMS analysis confirmed preparation of the desired product compound. 48.99%; ESI MS m/z calcd. For C₁₈H₂₆Cl₂N₂O₄S ([M+H]⁺) 437.38; found 437.25;

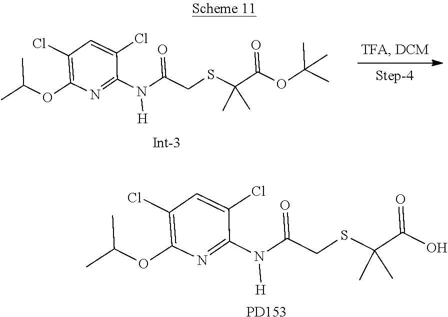

### Materials and Methods

To a stirred solution of tert-butyl 2-[2-[(3,5-dichloro-6-isopropoxy-2-pyridyl)amino]-2-oxo-ethyl]sulfanyl-2-methyl-propanoate, Int-3 (150 mg, 0.22 mmol, 1.0 eq) in DCM (5 mL), TFA (2 mL) was added at 0° C. The resulting reaction mixture was stirred for 4 h at room temperature. Progress of reaction was monitored by TLC. After complete conversion of starting material, the reaction mixture was evaporated to get crude compound. The crude compound was purified by Prep HPLC to afford 2-[2-[(3,5-dichloro-6-isopropoxy-2-pyridyl)amino]-2-oxo-ethyl]sulfanyl-2-methyl-propanoic acid, compound 4 (PD153) (35 mg, 39%) as an off white solid.

NMR, LCMS, and HPLC analyses confirmed preparation of the desired product compound. ¹H NMR (400 MHz, DMSO-d6) δ 12.8 (br, 1H) 10.4 (br, 1H) 8.12 (s, 1H) 5.19-5.13 (m, 1H) 3.57 (s, 2H) 1.43 (s, 6H) 1.32-1.30 (d, 6H). LCMS: 99.12%; ESI MS m/z calcd. For C14H18C12N2O4S ([M+) 381.27; found 381.0; HPLC: 98.15%.

### Example 5: Preparation of 2-((2-((3,5-dichloro-6-isopropoxypyridin-2-yl)amino)-2-oxoethyl)thio)-2-methylpropanoic acid (PD154; Compound 60)

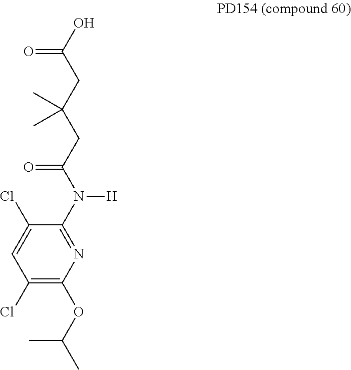 [SMILES: CC(C)Oc1nc(NC(=O)CC(C)(C)CC(=O)O)c(Cl)cc1Cl]

This example illustrates the preparation of the FABP 4/5 inhibitor compound 60 (PD154) via the synthetic method of Scheme 12 as shown below.

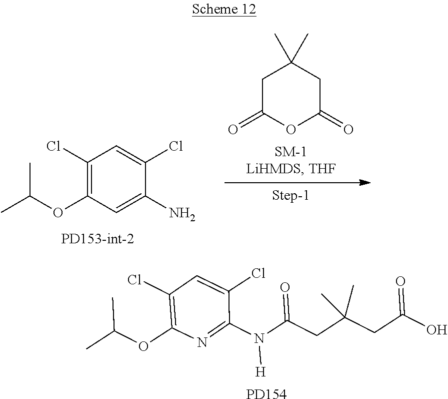

### Materials and Methods

To a stirred solution of 3,5-dichloro-6-isopropoxy-pyridin-2-amine, Int-2 (100 mg, 0.45 mmol, 1.0 eg) in THF (5 mL), LiHMDS (0.9 mL, 0.90 mmol, 2.0 eq) was added at 0° C. and the resultant reaction mixture stirred for 30 minutes. And then 4,4-dimethyltetrahydropyran-2,6-dione SM-1(128 mg, 0.90 mmol, 2.0 eq) was added and the resulting reaction mixture was stirred for 16 h at room temperature. Progress of the reaction was monitored by TLC. After complete completion of the starting materials, the reaction mixture was quenched with saturated Ammonium Chloride solution and extracted with ethyl acetate (2×30 mL). The combined organic extracts were washed with water (30 mL) and then brine solution (30 mL) and dried over anhydrous sodium sulphate, filtered, and concentrated under reduced pressure to obtain crude compound, which was purified by combi-flash chromatography, eluting with 0-5% MeOH in DCM to afford 5-[(3,5-dichloro-isopropoxy-2-pyridyl)amino]-3,3-dimethyl-5-oxo-pentanoic acid, compound 60 (65 mg, 39%) as a white solid.

NMR, LCMS, and HPLC analyses confirmed preparation of the desired product compound. ¹H NMR (400 MHz, DMSO-d₆) δ 12.01 (br, 1H) 10.05 (br, 1H) 8.12 (s, 1H) 5.16-5.13 (m, 1H) 2.40 (s, 2H) 2.33 (s, 2H) 1.32-1.30 (d, 6H) 1.11 (s, 6H). LCMS: 99.39%; ESI MS m/z calcd. For C15H20Cl2N2O4 [M+H]+ 363.24; found 363.1; HPLC: 99.17%.

### Example 6: Preparation of 2-((2-((3,5-dichloro-6-isopropoxypyridin-2-yl)amino)-2-oxoethyl)thio)-2-methylpropanoic acid (PD155; Compound 35)

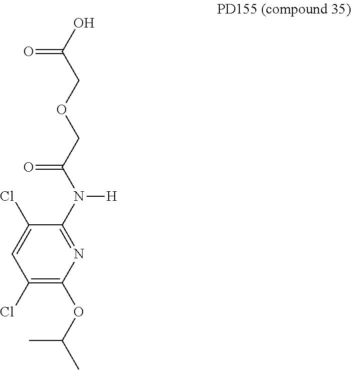 [SMILES: CC(C)Oc1nc(NC(=O)COCC(=O)O)c(Cl)cc1Cl]

This example illustrates the preparation of the FABP 4/5 inhibitor compound 35 (PD155) via the synthetic method of Scheme 13 as shown below.

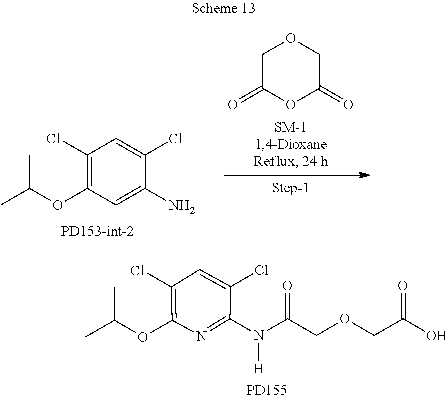

### Materials and Methods

To a stirred solution of 3,5-dichloro-6-isopropoxy-pyridin-2-amine, Int-2 (200 mg, 0.90 mmol, 1.0 eq) in 1,4-Dioxane (5 mL), 1,4-dioxane-2,6-dione SM-1(210 mg, 1.80 mmol, 2.0 eq) was added and the resultant reaction mixture stirred at 110° C. for 24 h. Progress of the reaction was monitored by TLC. After complete completion of the starting materials, the reaction mixture was diluted with water (20 ml) and extracted with DCM (2×30 mL). The combined organic extracts were washed with brine solution (30 mL) and dried over anhydrous sodium sulphate, filtered, and concentrated under reduced pressure to obtain crude compound, which was purified by combi-flash chromatography, eluting with 0-5% MeOH in DCM to afford 2-(2-((3,5-dichloro-6-isopropoxypyridin-2-yl)amino)-2-oxoethoxy)acetic acid, compound 35 (PD155) (50 mg, 16%) as a white solid.

NMR, LCMS, and HPLC analyses confirmed preparation of the desired product compound. ¹H NMR (400 MHz, DMSO-d6) δ 12.86 (br, 1H) 10.12 (br, 1H) 8.16 (s, 1H) 5.19-5.16 (m, 1H) 4.23 (s, 2H) 4.18 (s, 2H) 1.32-1.31 (d, 6H). LCMS: 99.69%; ESI MS m/z calcd. For C12H14Cl2N2O5 [M+H]+ 337.15; found 337.18; HPLC: 98.40%.

### Example 7: Screening of FABP3/4/5/7 Inhibitor Compounds

This example illustrates a two-step fluorescence binding assay study used to determine the binding affinity of the various inhibitor compounds disclosed herein with the various FABPs: FABP3, FABP4, FABP5 and FABP7. Further secondary screening studies were carried out for compounds with high binding affinities toward the FABPs and based on their ability to activate the nuclear receptors PPARα, PPARγ, or PPARδ.

### Materials and Methods

a. Binding Assays

Binding assays for FABP3, FABP4, FABP5 and FABP7 were carried out by fluorescence titrations. His-tagged FABPs were bacterially expressed in E. coli, purified using Ni Sepharose beads, and the equilibrium dissociation constants (Kd) that characterize their interactions with different inhibitor compounds were measured by fluorescence competition assays. The method entails two steps as described in e.g., Lin, Q. et al., “Ligand selectivity of the peroxisome proliferator-activated receptor alpha,” Biochemistry 38, 185-190, doi:10.1021/bi9816094 [pii](1999). In the first step, Kd for the association of the protein with the fluorescent fatty acid probe ANS was measured. Protein (2 μM) was titrated with ANS from a concentrated solution in DMSO. Ligand binding was monitored by following the increase in the fluorescence of the ligand upon binding to the protein, and Kd for the association of ANS with the each FABP was computed from titration curves as described in e.g., Norris, A. W. & Li, E., “Fluorometric titration of the CRABPs,” Methods Mol Biol 89, 123-139 (1998)). In the second step, Kds for binding of non-fluorescent ligands were measured by monitoring their ability to displace ANS in the binding pocket of the protein. Each FABP was precomplexed with ANS at 1:1 molar ratio and titrated with the different compounds whose binding was reflected by a decrease in probe fluorescence. Kds were extracted from the EC₅₀ of the competition curve and the measured Kd for ANS.

### B. Transcriptional Activation Assays

COS-7 were cultured in 6-well plates and co-transfected with either a luciferase reporter driven by 3 copies of a PPRE and expression vector for either PPARδ, PPARα or PPARγ together with a vector harboring cDNA for β-galactosidase, serving as a transfection control. To test whether FABP4 or FABP5 mediate activation of their cognate receptors PPARγ and PPARδ, respectively, cells were also co-transfected with a plasmid harboring sequence of either FABP4 or FABP5. 18 h post-transfection, cells were placed in a serum-free medium and treated with agonist/compound. 18 h. later, cells were lysed, luciferase activity was assayed (Promega, WI, USA) and corrected for transfection efficiency by the activity of β-galactosidase.

### Results

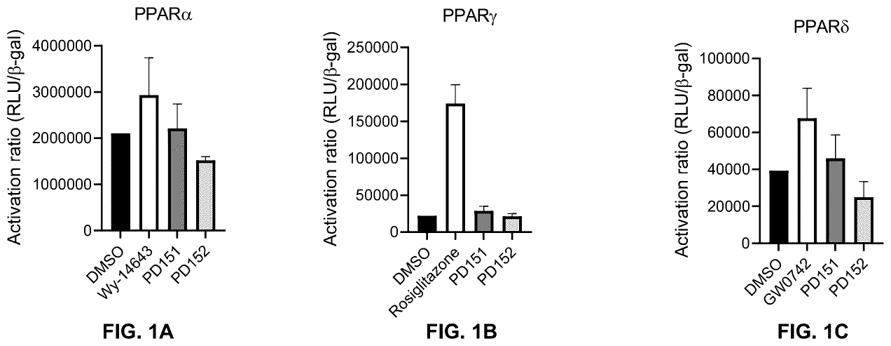

Results of the binding assays of FABP5, FABP4, FABP3, FABP7, FABP1, and FABP2 with the disclosed compounds PD151, PD152, and PD152.2, are summarized in Table 5 below. None of these tested compounds activated PPARα, PPARγ, or PPARδ (FIGS. 1A, 1B, and 1C).

TABLE 5

| | | FABP5 | FABP4 | FABP3 | FABP1 | FABP2 | FABP7 | |
|---|---|---|---|---|---|---|---|---|
| | | Kd | Kd | Kd | Kd | Kd | Kd | Mol |
| ID | Compound | (nM) | (nM) | (nM) | (nM) | (nM) | (nM) | Wt. |
| PD151 (compound 1) | 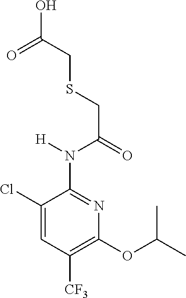 [SMILES: CC(C)Oc1nc(NC(=O)CSCC(=O)O)c(Cl)cc1C(F)(F)F] | 16 | 18 | 250 | ND* | ND* | >1000 | 387 |
| PD152 (compound 2) | 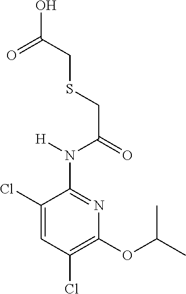 [SMILES: CC(C)Oc1nc(NC(=O)CSCC(=O)O)c(Cl)cc1Cl] | 28 | 18 | 188 | ND* | ND* | 989 | 353 |
| PD152.2 (compound 3) | 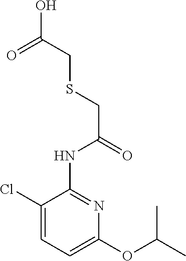 [SMILES: CC(C)Oc1ccc(Cl)c(NC(=O)CSCC(=O)O)n1] | 141 | 72 | 125 | NT** | NT** | 1000 | 319 |
| PD153 (compound 4) | 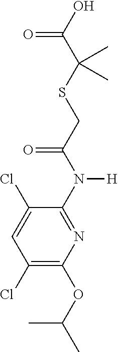 [SMILES: CC(C)Oc1nc(NC(=O)CSC(C)(C)C(=O)O)c(Cl)cc1Cl] | 80 | 96 | 98 | NT** | NT** | 344 | 381 |
| PD154 (compound 60) | 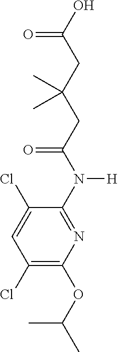 [SMILES: CC(C)Oc1nc(NC(=O)CC(C)(C)CC(=O)O)c(Cl)cc1Cl] | 11 | 14 | 60.8 | NT** | NT** | 43 | 363 |
| PD155 (compound 35) | 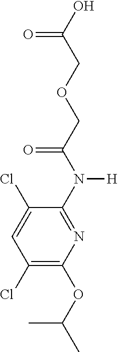 [SMILES: CC(C)Oc1nc(NC(=O)COCC(=O)O)c(Cl)cc1Cl] | 59 | 79 | 117 | NT** | NT** | 108 | 337 |

ND*, Not detected;
NT**, Not tested.

### Example 8: Biological Studies of FABP3/4/5/7 Inhibitor Compounds PD151 and PD152

This example illustrates studies of the biological function of the FABP3/4/5/7 inhibitor compound 1 (PD151) and compound 2 (PD152) in cancer models and in models of metabolic diseases.

### Materials and Methods

### A. Cells

COS-7, MDA-MB-231, and HepG2, were cultured in Dulbecco's modified Eagle's medium (DMEM) supplemented with 10% fetal bovine serum (Invitrogen Life Sciences, Carlsbad, CA). BT-549, OVCAR5 and OVCAR8 cells were cultured in L-Glutamine containing RPMI medium supplemented with 10% fetal calf serum (Invitrogen Life Sciences, Carlsbad, CA). The primary Human Mammary Epithelial Cells (HMEC) were cultured in mammary epithelial cell complete medium (basal medium plus rH-Insulin (5 μg/mL); L-Glutamine (6 mM); Epinephrine (1 μM); Apo-Transferrin (5 μg/mL); rH-TGF-α (0.5 ng/mL); ExtractP (0.4%); Hydrocortisone Hemisuccinate (100 ng/mL)).

### B. Transcriptional Activation Assays

Transcriptional activation assays were carried out utilizing COS7 cells as described above.

### C. Real-Time PCR

Cells were treated with a compound for 6 h. then lysed and RNA was extracted using Trizol, according to the manufacturer's instructions. cDNA was generated using GeneAmp RNA PCR (Applied Biosystems). qPCR was carried out using TaqMan chemistry and Assays-on-Demand probes (Applied Biosystems). 18s (4352930) rRNA was used for normalization. Relative expression was calculated as 2′^{DDCT}

### D. Proliferation Assays

2000 cells were plated in each well of 96-well plate. The next day cells were treated with compounds and incubated in Incucyte for 4 days. Images taken every 4 hours for the duration of the time were analyzed to calculate percentage of confluency in response to the treatments. Growth inhibition was calculated as 1—(percentage of viable cells out of untreated cells).

### E. Lipid Uptake Assays

HepG2 cells: 5000 cells were plated in 96-well plate. The next day cells were treated with tested compounds for 4 hours and then treated with 1 mM oleic acid for additional 24 hours. Cells were then stained with Nile Red and lipid content was measured using Spectra Max i3X plate reader (Molecular Devices) and quantified with the SoftMax Pro 6 software.

### F. Single Dose IV and PO PK Study in C57BL/6 Mice and Han Wistar Rats

C57BL/6 male mice or Han Wistar rats were used, n=3 for each administration route and compound. Single dose of each compound was administered orally (PO, 10 mg/Kg) or by intravenous administration (IV, 1 mg/Kg). Following intravenous and oral administration, blood samples (ca 0.2 mL) were collected and compound amount was measured by HLPC. For IV group, blood samples were collected at 0.083, 0.25, 0.5, 1, 2, 4, 6, 8, and 24 hours post-dose. For PO group, blood samples were collected at 0.25, 0.5, 1, 2, 4, 6, 8, and 24 hours post-dose.

### G. Xenograft Experiment

Seven-week-old Nu/Nu athymic female mice were injected subcutaneously into the right flank with 5×10⁶ MB-231 cells 1:1 with Matrigel. Once tumors reached ˜50 mm³ in size, mice were divided into 3 groups (n=5 for each group) and treatment started. Mice were treated by gavage with either vehicle (0.5% methyl cellulose, 1% Tween 80), 20 mg/kg, or 40 mg/kg of PD152 dissolved in vehicle, 5 days a week. Thirty days after treatment started mice were scarified. Statistical significance between the different treatment groups was evaluated using a Student's t-test. The experiment was conducted after approval by the institutional animal care and use committee at Case Western Reserve University.

### H. Type-1 Diabetes (T1D) Mouse Model

Female NOD/ShiLtJ mice were purchased from Jackson's lab at age of 7 weeks. Mice were housed in a temperature-controlled facility (23° C., 12-h light/dark cycle). Weekly non-fasting blood glucose concentrations were measured in the morning by tail vein prick starting at 8 weeks of age by using a blood glucose test meter (OTUltra2 system, Lifescam, USA) and strips according to manufacturer's instruction. Any reading of blood glucose higher than 200 mg/dL was confirmed by another test 24 hours later. Diabetes was diagnosed by 2 consensus positive blood glucose tests higher than 200 mg/dL. At 13 weeks of age, mice were divided into 3 treatments groups (n=15/group) as followed: 1) control group, treated with vehicle (0.5% methyl cellulose, 1% Tween 80); 2) PD152 treated group (40 mg/Kg in vehicle); 3) anti-CD3 treated group (5 μg/mouse, in PBS). Groups 1 and 2 were treated by gavage 5 days a week throughout the duration of the experiment. Group 3 was treated by IV injection for only 5 consecutive days. Mice were sacrificed at the age of 27 weeks.

### Results

a. Compounds PD151, and PD152 do not Activate Transcription by PPARs

To eliminate the possibility that the pyridine compounds PD151 and PD152 are ligands of the nuclear receptor PPARα, PPARγ, or PPARδ, that activate transcription by these transcription factors, transcriptional activation assays were conducted utilizing COS7 cells. For comparison, the known specific PPARα, PPARγ, or PPARδ agonist compounds, Wy-14643 (FIG. 1A), rosiglitazone (FIG. 1B), and GW0742 (FIG. 1C) (5 mM for all), respectively, were also assayed. As shown by the plots of data in FIG. 1A, FIG. 1B, and FIG. 1C, unlike these known agonist compounds which induced activation of their respective receptors, none of the tested compounds PD151 and PD152 activated transcription.

B. Compounds PD151 and PD152 Suppress Growth of TNBC Cells that Express High Levels of FABP5 but not the Normal Human Mammary Epithelial Cells

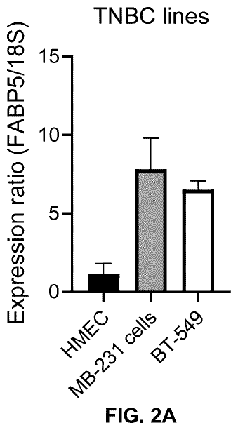

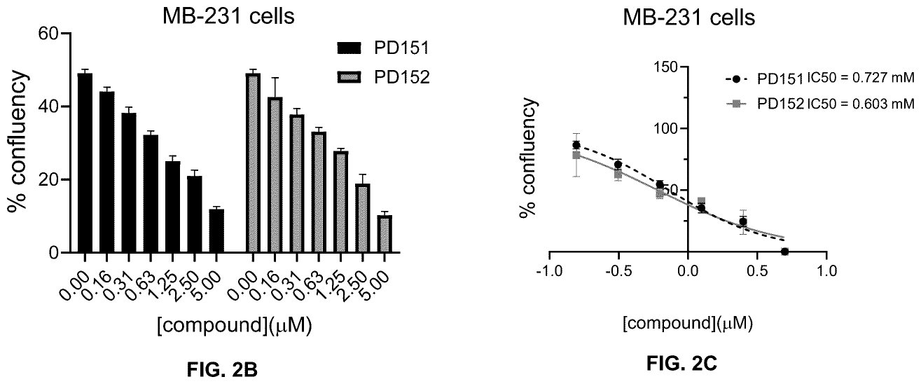

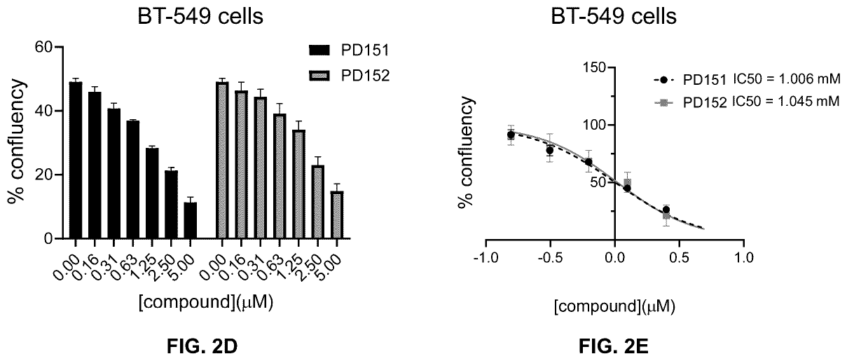

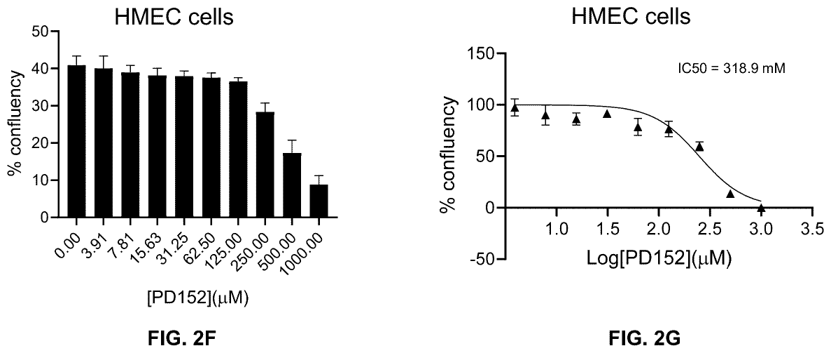

The TNBC lines MB-231 and BT-549 and the normal human mammary epithelial cells HMEC that express different levels of FABP5 (FIG. 2A) were used to test the effect of the compounds PD151 and PD152 on cell proliferation and to calculate their inhibition efficacy. As can be seen in FIG. 2A, the TNBC lines MB-231 and BT-549 express very high levels of FABP5 compared to HMEC cells. Cells were treated with serial dilution of the compound and proliferation was measured in Incucyte by calculating percentage of confluency every 4 hours over 4 days. As shown in FIG. 2B, FIG. 2C, FIG. 2D, and FIG. 2E, PD151 and PD152 efficiently inhibited proliferation of the 2 human TNBC lines. The calculated IC₅₀ for PD151 are 0.727 μM, and IC₅₀ of 1.006 μM, and for PD152 are 0.603 μM, and IC₅₀ of 1.045 μM, for MB-231, BT-549, respectively. To verify the inhibitory effect of PD152 on TNBC cells is FABP5-dependent and to test its safety to normal cells, the effect of the compound on cell proliferation was tested on the normal HMEC cells that express low FABP5 levels (FIG. 2A). As shown in FIG. 2F and FIG. 2G, PD152 inhibited proliferation of HMEC cells only when treated in very high concentrations (>125 μM). The calculated IC₅₀ for PD152 in HMEC cells is 318.9 μM which is 532- and 305-fold lower efficacy than the in MB-231 and BT-549 cells, respectively.

### C. Compounds PD151 and PD152 Suppress Growth of Human Ovarian Cancer Cells OVCAR5 and OVCAR8

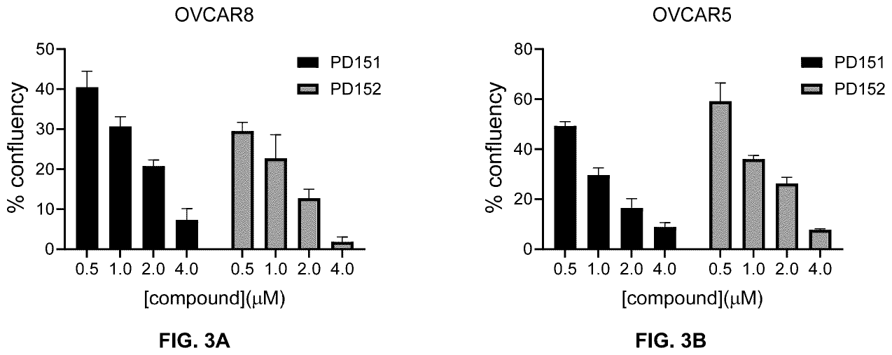

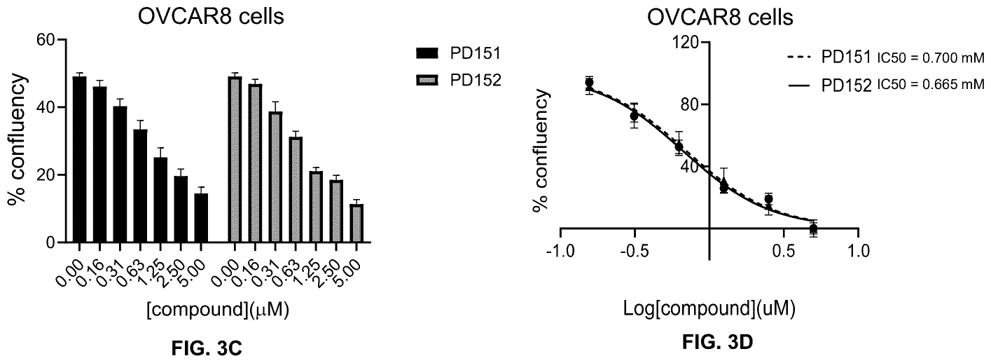

The ovarian cancer cell lines OVCAR5 and OVCAR8 were used to calculate the efficacy of compounds PD151 and PD152 in inhibiting proliferation of the ovarian cancer cells. Cells were treated with serial dilutions of the compounds and proliferation was measured in Incucyte by calculating percentage of confluency every 4 hours over 4 days. As shown in FIG. 3A FIG. 3B and FIG. 3C, the compounds inhibited proliferation of the two cell lines in a dose-dependent manner. As shown in FIG. 3D calculated IC₅₀ for inhibition of OVCAR8 proliferation by PD151 and PD152 are 0.7 μM and 0.665 μM, respectively.

D. PD152 Inhibits Uptake of Lipids into Hepatocytes in In Vitro Model for Liver Steatosis

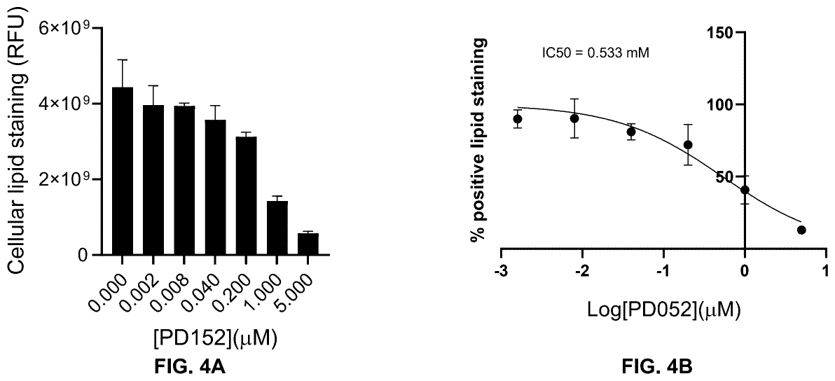

HepG2 cells were used to test the effect of PD152 on uptake of lipid into hepatic cells in an in vitro liver steatosis model. Cells were treated with oleic acid (OA) (1 mM) in the presence or absence of the pyridine compound and lipid accumulation in the cells was quantified using Nile Red. Uptake of lipid into hepatic cells treated with PD152 was markedly inhibited (FIG. 4A) in treatment concentrations >0.2 μM). As shown in FIG. 4B, Calculated IC50 for inhibition of lipid uptake by PD152 is 0.553 μM.

### E. PD151 and PD152 Exhibit Low Clearance, Rapid Oral Absorption and High Bioavailability

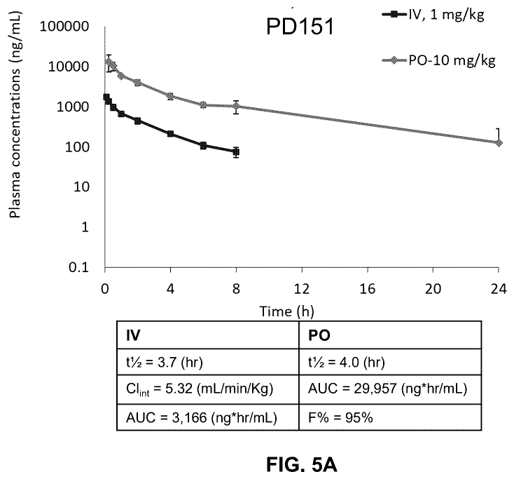

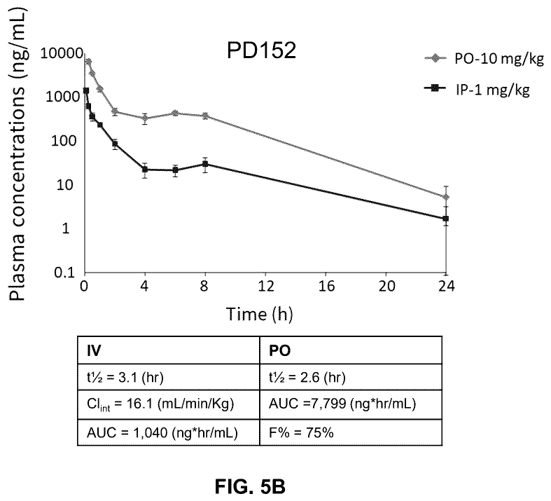

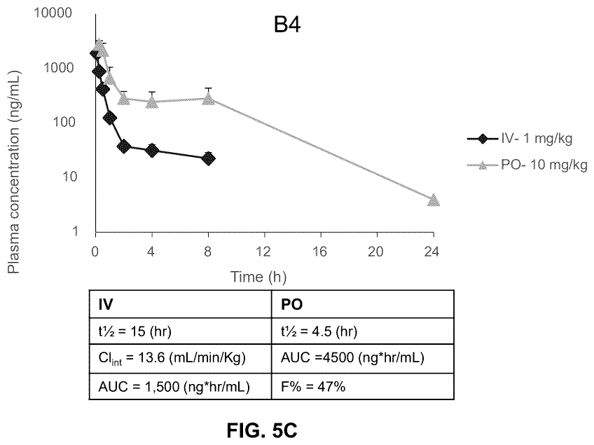

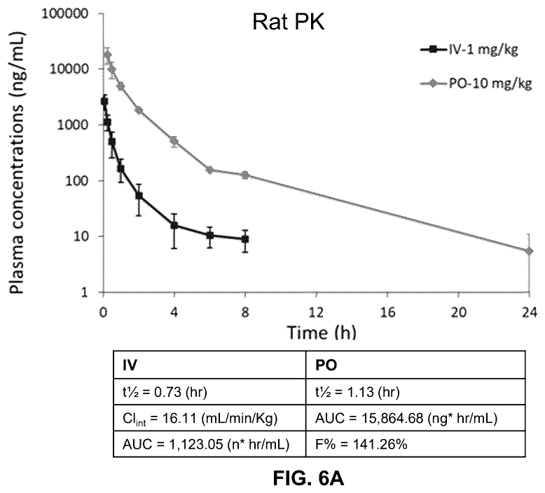

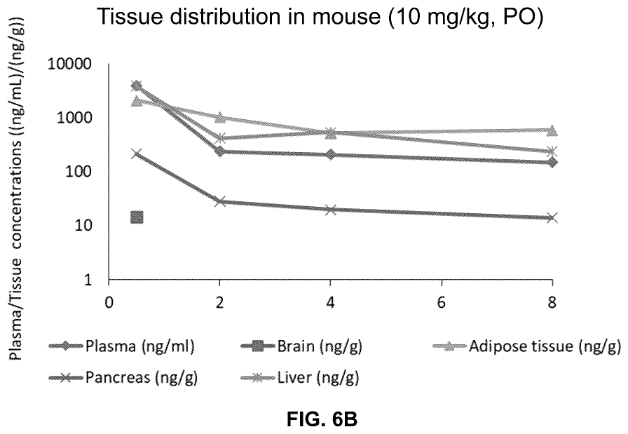

Single-dose oral administration of PD151 (FIG. 5A) and PD152 (FIG. 5B) at 10 mg/kg showed high bioavailability in male C57BL/6 mice (95%, and 75%, respectively) (FIG. 5). The measured bioavailability of these compounds is highly improved compared to the bioavailability measured for the aniline FABP4/5 inhibitor, 2-((2-((2,4-dichloro-5-isopropoxyphenyl)amino)-2-oxoethyl)thio)acetic acid (compound “B4” as disclosed in WO2023043803A1) (47%) (FIG. 5C). At 10 mg/kg, T_{half} was 4 and 2.6 hours, respectively, indicating rapid oral absorption, Intravenous administration of PD151 and PD152 at 1 mg/kg showed low plasma clearance of 5.3 mL/minute/kg (FIG. 5A) and 16.1 mL/minute/kg (FIG. 5B), respectively, and half-life of about 3-4 hours for both compounds, indicating low plasma clearance. Single-dose administration of PD152, orally and by intravenous injections in Han Wistar rats show similar results (FIG. 6A). Bioavailability at 10 mg/kg measured very high (141% (FIG. 6A) and T_{half} was 1.13 hours, indicating rapid oral absorption. Intravenous administration in rats at 1 mg/kg showed low plasma clearance of 16 mL/minute/kg (FIG. 6A), and half-life of about 0.73 hours. Compound PD152 was further tested for tissue distribution in C57BL/6 male mice (FIG. 6B). Single-dose of 10 mg/kg was administrated orally and plasma, brain, white adipose, pancreas, skeletal muscle, and liver tissues were collected at 0.5, 2, 4, and 8 hours after treatment and compound amount was measured by HPLC, As can be seen from the results sunmarized in Table 6, mean peak plasma, adipose tissue, liver, skeletal muscle, and pancreas concentration were found to be 3929.88 ng/mL, 2109.6 ng/g, 3893.5 ng/g, 139.45 ng/g. 139.45 ng/g, and 215.09 ng/g, respectively. Only traces of PD152 were detected in the brain indicating this compound does not cross the blood-brain barrier.

TABLE 6

| | | | | | | Skeletal |
|---|---|---|---|---|---|---|
| Time | Plasma | Brain | Adipose | Pancreas | Liver | muscle |
| (hrs) | (ng/ml) | (ng/g) | tissue (ng/g) | (ng/g) | (ng/g) | (ng/g) |
| 0.5 | 3929.9 | 14.8 | 2109.6 | 215.1 | 3893.50 | 139.45 |
| 2.0 | 236.2 | 0.0 | 1024.9 | 28.2 | 419.07 | 26.53 |
| 4.0 | 209.5 | 0.0 | 520.3 | 20.0 | 533.82 | 14.01 |
| 8 | 149.2 | 0.0 | 595.5 | 14.0 | 238.13 | 21.08 |

Calculated tissue/plasma ratios (Kp) are 1.3, 1.3, 0.05 and 0.07 for adipose tissue, liver, skeletal muscle, and pancreas, respectively, are summarized in Table 7,

TABLE 7

| | | Tissue/Plasma ratio |
|---|---|---|
| Tissue | PK parameters (ng*h/g) | (Kp) |
| Brain | ND | ND |
| Adipose tissue | 6655.32 | 1.3 |
| Liver | 6704.59 | 1.3 |
| Skeletal Muscle | 270.09 | 0.05 |
| Pancreas | 352.54 | 0.07 |

F. PD152 does not Exhibit Genotoxicity or Cytotoxicity

PD152 (compound 2) was tested for genotoxic and cytotoxic side effects by Ames test. The Ames test is a Bacterial Reverse Mutation Test of mutagenicity that utilizes Salmonella typhimurium tester strains TA98, TA100, TA1535, TA1537 and Escherichia coli tester strain WP2 uvrA (pKM101), both in the presence and absence of S9.

The compound PD152 was tested at 10 concentrations (0.032-1000 μg/plate) in the presence of, or absence of, the hepatic enzyme S9. As shown by the results summarized in Tables 8 and 9, there was no significant increase in the mean number of revertants by 2-fold in the tested strains TA98, TA100, WP2 uvrA (pKM101) and 3-fold in tested strains TA1535, TA1537 during the Ames mutagenicity study, indicating there was no positive mutagenic responses with the strains tested, in either the presence or the absence of S9 activation. These results indicate that PD152 is not cytotoxic to the tested strains of TA98, TA100, TA1535, TA1537 and WP2 uvrA (pKM101), at the tested concentrations ranging from 0.032 up to 1000 μg/plate.

TABLE 8
Mutagenicity test results for compound PD152
in the presence of metabolic activation (S9)

| | |
|---|---|
| TA98 | TA100 |

| Treatment | | | Fold | | | Fold |
|---|---|---|---|---|---|---|
| (μg/plate) | Mean | SD | increase | Mean | SD | increase |
| Vehicle | 6 | 1 | NA | 39 | 2 | NA |
| 0.0312 | 6 | 1 | 1 | 40 | 2 | 1 |
| 0.1 | 5 | 2 | 1 | 40 | 1 | 1 |
| 0.316 | 6 | 2 | 1 | 40 | 2 | 1 |
| 1 | 6 | 2 | 1 | 39 | 2 | 1 |
| 3.16 | 6 | 1 | 1 | 40 | 2 | 1 |
| 10 | 6 | 1 | 1 | 39 | 3 | 1 |
| 31.6 | 6 | 1 | 1 | 40 | 2 | 1 |
| 100 | 6 | 1 | 1 | 38 | 4 | 1 |
| 316 | 6 | 1 | 1 | 40 | 1 | 1 |
| 1000 | 5 | 1 | 1 | 39 | 2 | 1 |
| PC | 97 | 4 | 16 | 144 | 7 | 4 |
| | TA1535 | | | TA1537 | | |
| Vehicle | 4 | 1 | NA | 4 | 1 | NA |
| 0.0312 | 3 | 2 | 1 | 4 | 1 | 1 |
| 0.1 | 4 | 1 | 1 | 3 | 1 | 1 |
| 0.316 | 3 | 2 | 1 | 3 | 2 | 1 |
| 1 | 4 | 1 | 1 | 3 | 1 | 1 |
| 3.16 | 4 | 1 | 1 | 3 | 1 | 1 |
| 10 | 4 | 1 | 1 | 4 | 2 | 1 |
| 31.6 | 4 | 2 | 1 | 3 | 1 | 1 |
| 100 | 4 | 1 | 1 | 3 | 1 | 1 |
| 316 | 4 | 1 | 1 | 4 | 1 | 1 |
| 1000 | 4 | 1 | 1 | 4 | 1 | 1 |
| PC | 95 | 8 | 26 | 84 | 9 | 23 |
| | E. coli | | | | | |
| Vehicle | 40 | 2 | NA | | | |
| 0.0312 | 39 | 3 | 1 | | | |
| 0.1 | 42 | 1 | 1 | | | |
| 0.316 | 40 | 2 | 1 | | | |
| 1 | 38 | 7 | 1 | | | |
| 3.16 | 38 | 7 | 1 | | | |
| 10 | 41 | 2 | 1 | | | |
| 31.6 | 41 | 3 | 1 | | | |
| 100 | 39 | 4 | 1 | | | |
| 316 | 40 | 2 | 1 | | | |
| 1000 | 40 | 3 | 1 | | | |
| PC | 155 | 10 | 4 | | | |

Note:
PC = Positive control, SD = Standard deviation, NA—Not Applicable; Fold Increase (FI) = (Mean of individual revertant colonies in the treated group)/(Mean of individual revertant colonies in the vehicle control group)

TABLE 9
Mutagenicity test results for compound PD152
in the absence of metabolic activation (S9)

| Treatment | TA98 | | | TA100 | | |
|---|---|---|---|---|---|---|
| (μg/plate) | Mean | SD | Fold increase | Mean | SD | Fold increase |
| Vehicle | 5 | 1 | NA | 39 | 1 | NA |
| 0.0312 | 5 | 2 | 1 | 39 | 2 | 1 |
| 0.1 | 6 | 1 | 1 | 39 | 3 | 1 |
| 0.316 | 6 | 2 | 1 | 40 | 3 | 1 |
| 1 | 6 | 2 | 1 | 39 | 2 | 1 |
| 3.16 | 6 | 1 | 1 | 39 | 3 | 1 |
| 10 | 6 | 1 | 1 | 40 | 1 | 1 |
| 31.6 | 5 | 2 | 1 | 40 | 2 | 1 |
| 100 | 6 | 1 | 1 | 39 | 2 | 1 |
| 316 | 6 | 2 | 1 | 40 | 1 | 1 |
| 1000 | 6 | 1 | 1 | 40 | 1 | 1 |
| PC | 88 | 3 | 17 | 153 | 10 | 4 |
| | TA1535 | | | TA1537 | | |
| Vehicle | 3 | 1 | NA | 3 | 1 | NA |
| 0.0312 | 3 | 1 | 1 | 3 | 2 | 1 |
| 0.1 | 3 | 2 | 1 | 3 | 1 | 1 |
| 0.316 | 3 | 1 | 1 | 4 | 2 | 1 |
| 1 | 3 | 2 | 1 | 3 | 1 | 1 |
| 3.16 | 3 | 2 | 1 | 3 | 1 | 1 |
| 10 | 4 | 1 | 1 | 3 | 2 | 1 |
| 31.6 | 4 | 1 | 1 | 4 | 1 | 1 |
| 100 | 4 | 1 | 1 | 3 | 2 | 1 |
| 316 | 3 | 1 | 1 | 3 | 1 | 1 |
| 1000 | 3 | 2 | 1 | 3 | 1 | 1 |
| PC | 83 | 13 | 28 | 91 | 9 | 34 |
| | E.coli | | | | | |
| Vehicle | 41 | 2 | NA | | | |
| 0.0312 | 41 | 3 | 1 | | | |
| 0.1 | 39 | 3 | 1 | | | |
| 0.316 | 41 | 7 | 1 | | | |
| 1 | 39 | 2 | 1 | | | |
| 3.16 | 42 | 3 | 1 | | | |
| 10 | 40 | 7 | 1 | | | |
| 31.6 | 42 | 4 | 1 | | | |
| 100 | 42 | 3 | 1 | | | |
| 316 | 40 | 1 | 1 | | | |
| 1000 | 42 | 3 | 1 | | | |
| PC | 162 | 8 | 4 | | | |

Note:
PC = Positive control, SD = Standard deviation, NA—Not Applicable; Fold Increase (FI) = (Mean of individual revertant colonies in the treated group)/(Mean of individual revertant colonies in the vehicle control group)

The aniline FABP4/5 inhibitor compound, 2-((2-((2,4-dichloro-5-isopropoxyphenyl)amino)-2-oxoethyl)thio)-2-methylpropanoic acid (compound “B104” as disclosed in WO2023043803A1) also was tested for genotoxic and cytotoxic side effects by Ames test. B104 was tested using the Salmonella typhimurium tester stains TA98, TA100, TA1535, and TA1537, both in the presence and absence of the hepatic enzyme S9, at 5 concentrations (1.5-64 μg/plate). As shown by the results summarized in Tables 10 and 11, there were increases in the mean number of revertants in the tested strains TA98, TA100, TA1535, and TA1537 during the Ames mutagenicity study of B104, indicating positive mutagenic responses with the strains tested, in the presence or absence of S9 activation. These results indicate that B104 exhibits some level of cytotoxicity to the tested stains of TA98, TA100, TA1535, and TA1537, at the tested concentrations ranging from 0.032 up to 1000 μg/plate.

TABLE 10
Mutagenicity test results for compound B104
in the presence of metabolic activation (S9)

| | TA98 | | | TA100 | | |
|---|---|---|---|---|---|---|
| Treatment | | | Fold | | | Fold |
| (μg/well) | Mean | SD | increase | Mean | SD | increase |
| Vehicle | 17 | 2.52 | NA | 127.33 | 7.96 | NA |
| 1.5 | 20.67 | 1.20 | 1.2 | 387 | 1 | 3 |
| 4 | 39 | 6.51 | 2.3 | 658 | 17 | 5.18 |
| 10 | 49 | 2.52 | 2.88 | 849 | 58 | 6.68 |
| 25 | 47.67 | 5.04 | 2.7 | 833 | 77 | 6.55 |
| 64 | 26 | 4.51 | 1.5 | 564.5 | 4.5 | 4.44 |
| PC | 1850 | 656.2 | 108.8 | 974.5 | 1205.6 | 7.65 |
| | TA1535 | | | TA1537 | | |
| Vehicle | 6.33 | 1.67 | NA | 2.33 | 0.882 | NA |
| 1.5 | 5.67 | 0.67 | 0.89 | 4.0 | 0 | 1.71 |
| 4 | 6.33 | 1.45 | 1 | 5.5 | 0.5 | 2.36 |
| 10 | 6.67 | 0.88 | 1.06 | 3.0 | 3.0 | 1.29 |
| 25 | 10.67 | 0.67 | 1.68 | 2.5 | 1.5 | 1.07 |
| 64 | 6.33 | 1.2 | 1 | 5.0 | 0 | 2.14 |
| PC | 11.5 | 0.71 | 1.81 | 11.5 | 2.12 | 4.93 |

Note:
PC = Positive control, SD = Standard deviation, NA—Not Applicable; Fold Increase (FI) = (Mean of individual revertant colonies in the treated group)/(Mean of individual revertant colonies in the vehicle control group)

TABLE 11
Mutagenicity test results for compound B104
in the absence of metabolic activation (S9)

| | TA98 | | | TA100 | | |
|---|---|---|---|---|---|---|
| Treatment | | | Fold | | | Fold |
| (μg/well) | Mean | SD | increase | Mean | SD | increase |
| Vehicle | 12.67 | 2.03 | NA | 64.67 | 4.33 | NA |
| 1.5 | 16 | 1.73 | 1.26 | 52 | 5.77 | 0.8 |
| 4 | 10.67 | 2.96 | 0.84 | 68 | 3.21 | 1.05 |
| 10 | 12.33 | 0.33 | 0.97 | 56 | 4.51 | 0.86 |
| 25 | 0.33 | 0.33 | 0 | 0 | 0 | 0 |
| 64 | 0.66 | 0.33 | 0 | 0.67 | 0.67 | 0 |
| PC | 205.5 | 116.7 | 16.22 | 573.5 | 57.3 | 8.87 |
| | TA1535 | | | TA1537 | | |
| Vehicle | 3.33 | 2.33 | NA | 4.67 | 0.33 | NA |
| 1.5 | 4 | 1 | 1.20 | 2.0 | 0 | 0.43 |
| 4 | 7.33 | 1.45 | 2.20 | 1.5 | 1.5 | 0.32 |
| 10 | 4.67 | 1.45 | 1.40 | 1.0 | 0 | 0.21 |
| 25 | 0.67 | 0.67 | 0.20 | 0 | 0 | 0 |
| 64 | 0 | 0 | 0 | 0 | 0 | 0 |
| PC | 176 | 2.82 | 52.85 | 211 | 5.65 | 45.18 |

Note:
PC = Positive control, SD = Standard deviation, NA—Not Applicable; Fold Increase (FI) = (Mean of individual revertant colonies in the treated group)/(Mean of individual revertant colonies in the vehicle control group)

As shown by the results, the FABP4/5 inhibitor compound, PD152 (compound 2) is non-cytotoxic and non-mutagenic up to 1000 μg/plate both in the presence (Table 8) and absence (Table 9) of the metabolic activation system (S9). Thus, PD152 represents a significant improvement over previous aniline FABP4/5 inhibitor compounds, such as B104, that exhibit genotoxicity in an Ames test (see e.g., Tables 10 and 11).

### G. PD152 Suppresses Growth of TNBC Tumors in an In Vivo Xenograft Model

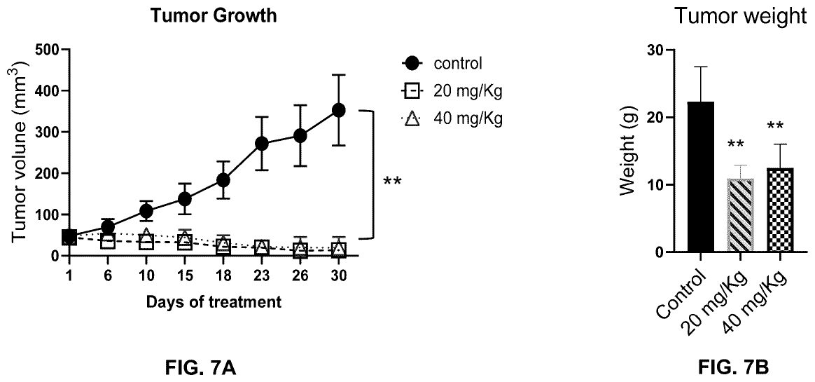

TNBC xenograft model was used to test the efficacy of PD152 in suppressing tumors growth in vivo. 5×10⁶ MB-231 cells were subcutaneously injected into the right flank of Nu/Nu nude mice and tumor growth was monitored twice a week. Once tumors were established and reached volume of ˜50 mm³, mice were divided into 3 groups and treatments started. Mice were treated orally by either PD152 (20 mg/Kg, or 40 mg/Kg), or a vehicle 5 times a week. As shown by the results depicted in FIGS. 7A and 7B, PD152-treated tumors exhibited reduced volume over time and their growth was completely inhibited (FIG. 7A). This was also observed by tumors' weight that was significantly lower for PD152-treated tumors (FIG. 7B).

### H. PD152 Treatment Reduces the Incidence of Type 1 Diabetes in NOD Mice.

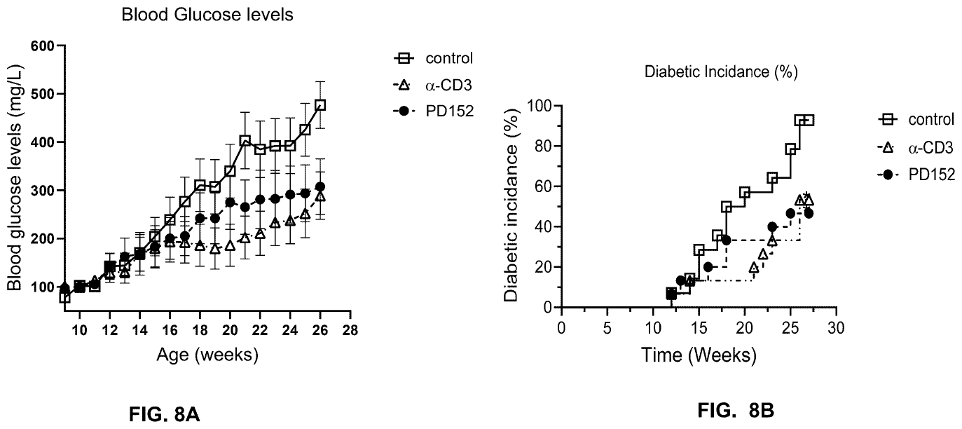

NOD mouse model of autoimmune type 1 diabetes was used to test the efficacy of FABP4/5 inhibitor PD152 on spontaneous development of the disease. Female mice at age of 13 weeks were divided into 3 groups (control, PD152, and anti-CD3 as positive control) and treatment started (see details in Methods). Blood glucose levels were measured weekly for monitoring the incidence of diabetes until termination at 27 weeks of age. A lower blood glucose levels in PD152-treated and anti-CD3-treated groups were observed starting at week 16 of age with small difference (3 weeks after treatment started) and reaching about 2-fold difference at 27 weeks of age (FIG. 8A). Consequently, approximately 92.9% of vehicle-treated NOD mice developed diabetes by 27 weeks of age, whereas diabetes incidence of PD152-treated mice was significantly inhibited to 46.7% (FIG. 8B).

### REFERENCES

1. Hardaway A L, Podgorski I: IL-1beta, RAGE and FABP4: targeting the dynamic trio in metabolic inflammation and related pathologies. Future Med Chem 2013, 5:1089-1108.
2. Furuhashi M, Hotamisligil G S: Fatty acid-binding proteins: role in metabolic diseases and potential as drug targets. Nat Rev Drug Discov 2008, 7:489-503.
3. Furuhashi M, Tuncman G, Gorgun C Z, Makowski L, Atsumi G, Vaillancourt E, Kono K, Babaev V R, Fazio S, Linton M F, et al: Treatment of diabetes and atherosclerosis by inhibiting fatty-acid-binding protein aP2. Nature 2007, 447:959-965.
4. Hotamisligil G S, Bernlohr D A: Metabolic functions of FABPs—mechanisms and therapeutic implications. Nat Rev Endocrinol 2015, 11:592-605.
5. Furuhashi M, Ishimura S, Ota H, Miura T: Lipid chaperones and metabolic inflammation. Int J Inflam 2011, 2011:642612.
6. Maeda K, Cao H, Kono K, Gorgun C Z, Furuhashi M, Uysal K T, Cao Q, Atsumi G, Malone H, Krishnan B, et al: Adipocyte/macrophage fatty acid binding proteins control integrated metabolic responses in obesity and diabetes. Cell Metab 2005, 1:107-119.
7. Levi L, Lobo G, Doud M K, von Lintig J, Seachrist D, Tochtrop G P, Noy N: Genetic ablation of the fatty acid binding protein FABP5 suppresses HER2-induced mammary tumorigenesis. Cancer Res 2013.
8. Levi L, Wang Z, Doud M K, Hazen S L, Noy N: Saturated fatty acids regulate retinoic acid signalling and suppress tumorigenesis by targeting fatty acid-binding protein 5. Nat Commun 2015, 6:8794.
9. Powell C A, Nasser M W, Zhao H, Wochna J C, Zhang X, Shapiro C, Shilo K, Ganju R K: Fatty acid binding protein 5 promotes metastatic potential of triple negative breast cancer cells through enhancing epidermal growth factor receptor stability. Oncotarget 2015, 6:6373-6385.
10. Li Y, Lee W, Zhao Z G, Liu Y, Cui H, Wang H Y: Fatty acid binding protein 5 is a novel therapeutic target for hepatocellular carcinoma. World J Clin Oncol 2024, 15:130-144.
11. Sun J, Esplugues E, Bort A, Cardelo M P, Ruz-Maldonado I, Fernandez-Tussy P, Wong C, Wang H, Ojima I, Kaczocha M, et al: Fatty acid binding protein 5 suppression attenuates obesity-induced hepatocellular carcinoma by promoting ferroptosis and intratumoral immune rewiring. Nat Metab 2024, 6:741-763.
12. Kralisch S, Fasshauer M: Adipocyte fatty acid binding protein: a novel adipokine involved in the pathogenesis of metabolic and vascular disease? Diabetologia 2013, 56:10-21.
13. Elmasri H, Ghelfi E, Yu C W, Traphagen S, Cernadas M, Cao H, Shi G P, Plutzky J, Sahin M, Hotamisligil G, Cataltepe S: Endothelial cell-fatty acid binding protein 4 promotes angiogenesis: role of stem cell factor/c-kit pathway. Angiogenesis 2012, 15:457-468.
14. Nieman K M, Kenny H A, Penicka C V, Ladanyi A, Buell-Gutbrod R, Zillhardt M R, Romero I L, Carey M S, Mills G B, Hotamisligil G S, et al: Adipocytes promote ovarian cancer metastasis and provide energy for rapid tumor growth. Nat Med 2011, 17:1498-1503.
15. Chew V, Toh H C, Abastado J P: Immune microenvironment in tumor progression: characteristics and challenges for therapy. J Oncol 2012, 2012:608406.
16. Aras S, Zaidi M R: TAMeless traitors: macrophages in cancer progression and metastasis. Br J Cancer 2017, 117:1583-1591.
17. Mantovani A, Marchesi F, Malesci A, Laghi L, Allavena P: Tumour-associated macrophages as treatment targets in oncology. Nat Rev Clin Oncol 2017, 14:399-416.
18. Kobayashi S, Wannakul T, Sekino K, Takahashi Y, Kagawa Y, Miyazaki H, Umaru B A, Yang S, Yamamoto Y, Owada Y: Fatty acid-binding protein 5 limits the generation of Foxp3(+) regulatory T cells through regulating plasmacytoid dendritic cell function in the tumor microenvironment. Int J Cancer 2022, 150:152-163.
19. Jin R, Hao J, Yu J, Wang P, Sauter E R, Li B: Role of FABP5 in T Cell Lipid Metabolism and Function in the Tumor Microenvironment. Cancers (Basel) 2023, 15.
20. Pan Y, Kupper T S: Metabolic Reprogramming and Longevity of Tissue-Resident Memory T Cells. Front Immunol 2018, 9:1347.
21. Wu H, Liao W, Li Q, Long H, Yin H, Zhao M, Chan V, Lau C S, Lu Q: Pathogenic role of tissue-resident memory T cells in autoimmune diseases. Autoimmun Rev 2018, 17:906-911.
22. Pan Y, Tian T, Park C O, Lofftus S Y, Mei S, Liu X, Luo C, O'Malley J T, Gehad A, Teague J E, et al: Survival of tissue-resident memory T cells requires exogenous lipid uptake and metabolism. Nature 2017, 543:252-256.
23. Yenyuwadee S, Sanchez-Trincado Lopez J L, Shah R, Rosato P C, Boussiotis V A: The evolving role of tissue-resident memory T cells in infections and cancer. Sci Adv 2022, 8:eabo5871.
24. Richmond J M, Strassner J P, Rashighi M, Agarwal P, Garg M, Essien K I, Pell L S, Harris J E: Resident Memory and Recirculating Memory T Cells Cooperate to Maintain Disease in a Mouse Model of Vitiligo. J Invest Dermatol 2019, 139:769-778.
25. Molodtsov A K, Khatwani N, Vella J L, Lewis K A, Zhao Y, Han J, Sullivan D E, Searles T G, Preiss N K, Shabaneh T B, et al: Resident memory CD8(+) T cells in regional lymph nodes mediate immunity to metastatic melanoma. Immunity 2021, 54:2117-2132 e2117.
26. Ryan G E, Harris J E, Richmond J M: Resident Memory T Cells in Autoimmune Skin Diseases. Front Immunol 2021, 12:652191.
27. Jin R, Hao J, Yi Y, Sauter E, Li B: Regulation of macrophage functions by FABP-mediated inflammatory and metabolic pathways. Biochim Biophys Acta Mol Cell Biol Lipids 2021, 1866:158964.
28. Zhang Y, Sun Y, Rao E, Yan F, Li Q, Zhang Y, Silverstein K A, Liu S, Sauter E, Cleary M P, Li B: Fatty acid-binding protein E-FABP restricts tumor growth by promoting IFN-beta responses in tumor-associated macrophages. Cancer Res 2014, 74:2986-2998.
29. Zeng J, Zhang Y, Hao J, Sun Y, Liu S, Bernlohr D A, Sauter E R, Cleary M P, Suttles J, Li B: Stearic Acid Induces CD11c Expression in Proinflammatory Macrophages via Epidermal Fatty Acid Binding Protein. J Immunol 2018, 200:3407-3419.
30. Zhang Y, Li Q, Rao E, Sun Y, Grossmann M E, Morris R J, Cleary M P, Li B: Epidermal Fatty Acid binding protein promotes skin inflammation induced by high-fat diet. Immunity 2015, 42:953-964.
31. Ma X, Bi E, Lu Y, Su P, Huang C, Liu L, Wang Q, Yang M, Kalady M F, Qian J, et al: Cholesterol Induces CD8(+) T Cell Exhaustion in the Tumor Microenvironment. Cell Metab 2019, 30:143-156 e145.
32. Baazim H, Koyuncu E, Tuncman G, Burak M F, Merkel L, Bahour N, Karabulut E, Lee G Y, Hanifehnezhad A, Karagoz Z F, et al: FABP4 as a Therapeutic Host Target Controlling SARS-CoV2 Infection. bioRxiv 2024:2024.2002.2010.579717.

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the purview of this disclosure and the appended claims. Further, one of skill in the art will recognize a number of equivalent embodiments, methods and procedures to those described herein. All such equivalents are to be understood to be within the scope of the present disclosure and are covered by the appended claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

What is claimed is:

1. A compound of structural formula I or a pharmaceutically acceptable salt thereof, 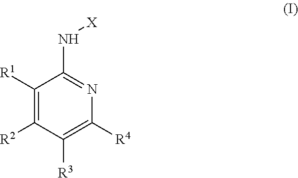
wherein,
R¹, R², R³, and R⁴ is each independently selected from hydrogen or a substitution for hydrogen;
X is a moiety of formula: 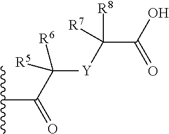
wherein,
Y is a heteroatom selected from —S—, —O—, —SO—, —SO₂—, or is —CR⁹R¹⁰—, wherein R⁹ and R¹⁰ are each independently selected from hydrogen, C₁-C₄ linear or branched alkyl, phenyl, and benzyl, or R⁹ and R¹⁰ together form a cyclopropyl, cyclobutyl, or cyclopentyl ring;
R⁵, R⁶, R⁷, and R⁸ is each independently selected from hydrogen, C₁-C₄ linear or branched alkyl, phenyl, and benzyl, and/or
R⁵ and R⁶ together or R⁷ and R⁸ together form a cyclopropyl ring, a cyclobutyl ring, a cyclopentyl ring, or an oxetane ring,
R⁶ and R⁷ together form a 4-, 5-, or 6-membered carbocyclic or heterocyclic ring with Y as a member of the ring, or
R⁵ and R⁶ together and R⁷ and R⁸ together form a bicyclo[1.1.1] ring.

2. The compound of claim 1, wherein R¹, R², R³, and R⁴ is each independently selected from hydrogen, chlorine, fluorine, tri-fluoromethyl, methoxy, difluoro-methoxy, trifluoro-methoxy, ethoxy, propoxy, isopropyloxy, cyclopropyloxy, propargyloxy, butoxy, iso-butoxy, sec-butoxy, tert-butoxy, 2-methoxyethoxy, 2-ethoxy-2-oxoethoxy, (3-methyloxetan-3-yl)oxy, or benzyloxy.

3. The compound of claim 1, wherein:
R¹ is selected from chlorine, fluorine, methyl, and tri-fluoromethyl;
R² is hydrogen;
R³ is selected from hydrogen, chlorine, fluorine, methyl, and tri-fluoromethyl; and
R⁴ is each independently selected from methoxy, difluoro-methoxy, trifluoro-methoxy, ethoxy, propoxy, isopropoxy, cyclopropoxy, propargyloxy, butoxy, iso-butoxy, sec-butoxy, tert-butoxy, 2-methoxyethoxy, 2-ethoxy-2-oxoethoxy, (3-methyloxetan-3-yl)oxy, or benzyloxy.

4. The compound of claim 1, wherein the compound of structural formula I has a structural formula selected from Ia, Ib, Ic, Id, Ie, If, Ig, Ih, Ii, Ij, Ik, Il, Im, In, Io, Ip, Iq, Ir, Is, It, Iu, Iv, Iw, Ix, Iy, Iz, Iaa, Ibb, Icc, and Idd:

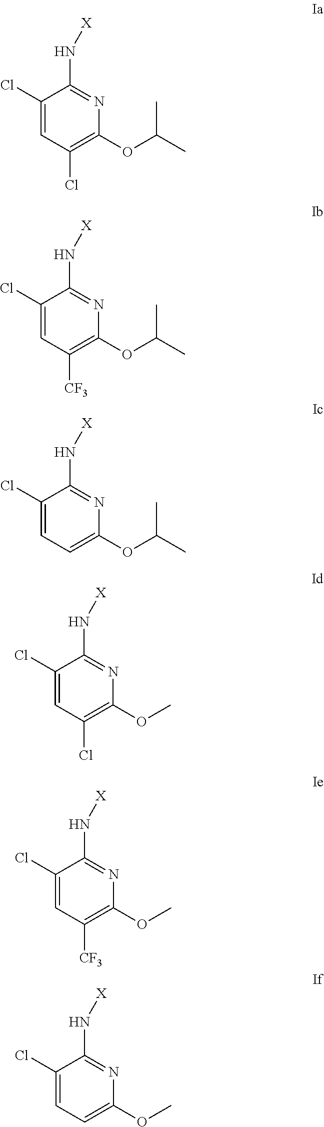

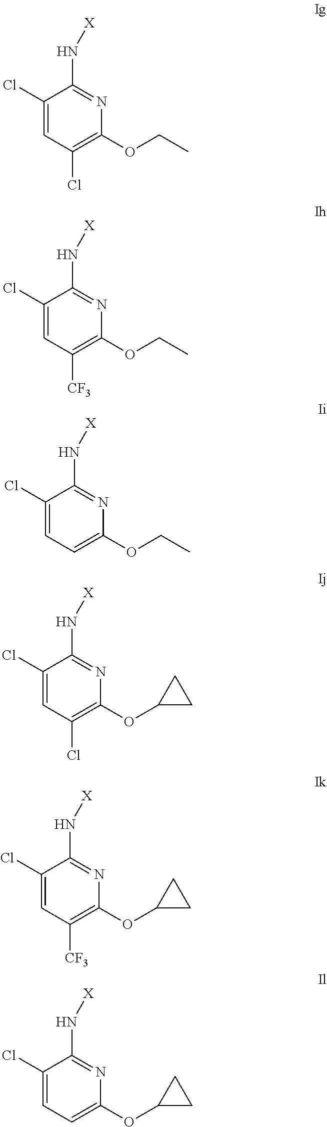

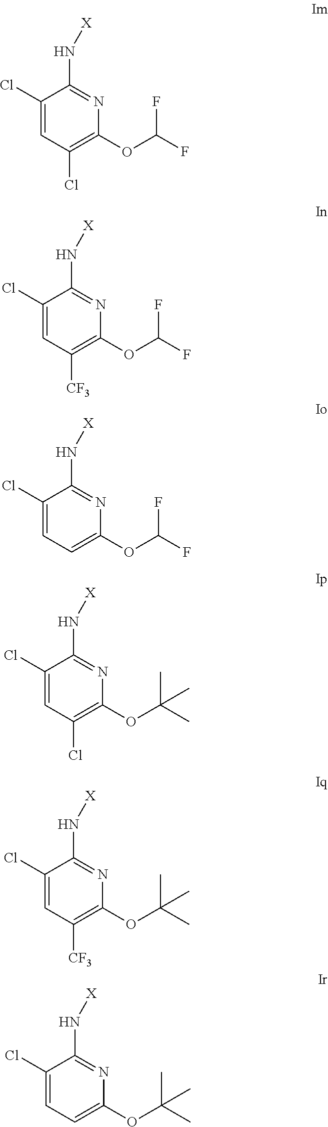

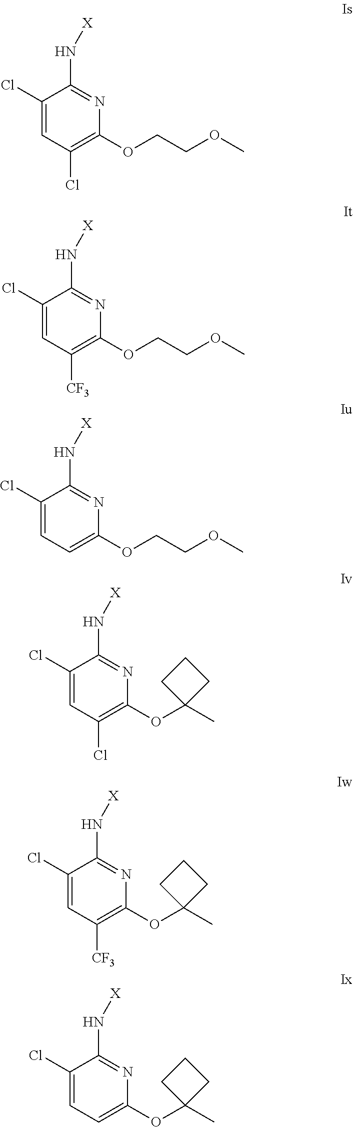

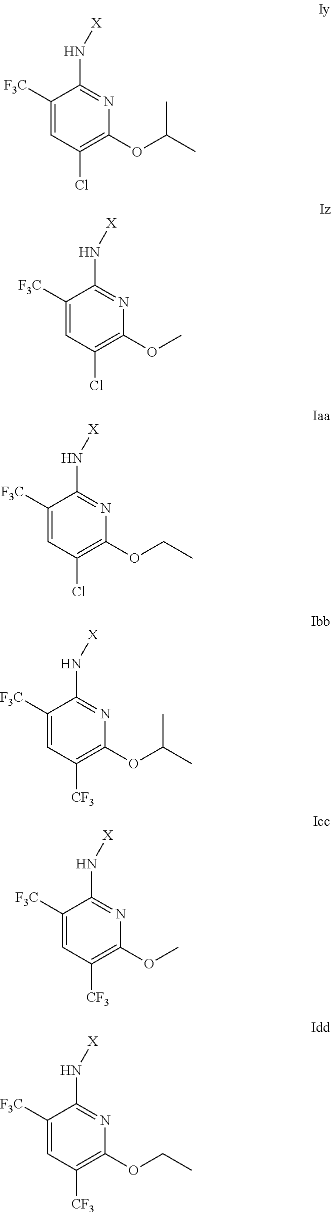

5. The compound of claim 1, wherein the X moiety is selected from:

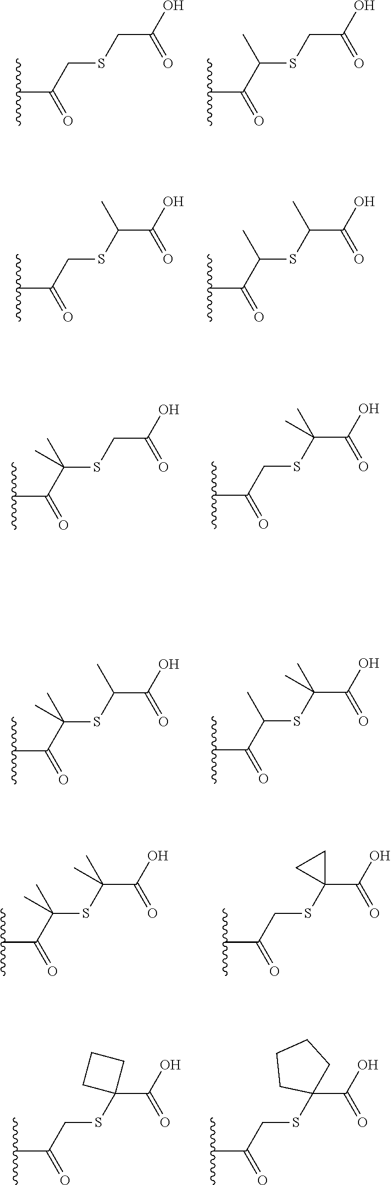

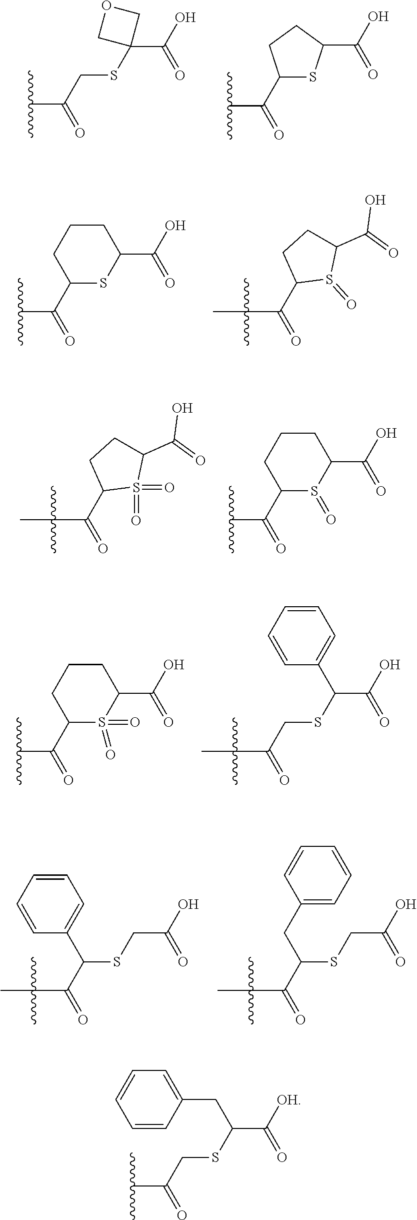

6. The compound of claim 1, wherein the X moiety is selected from:

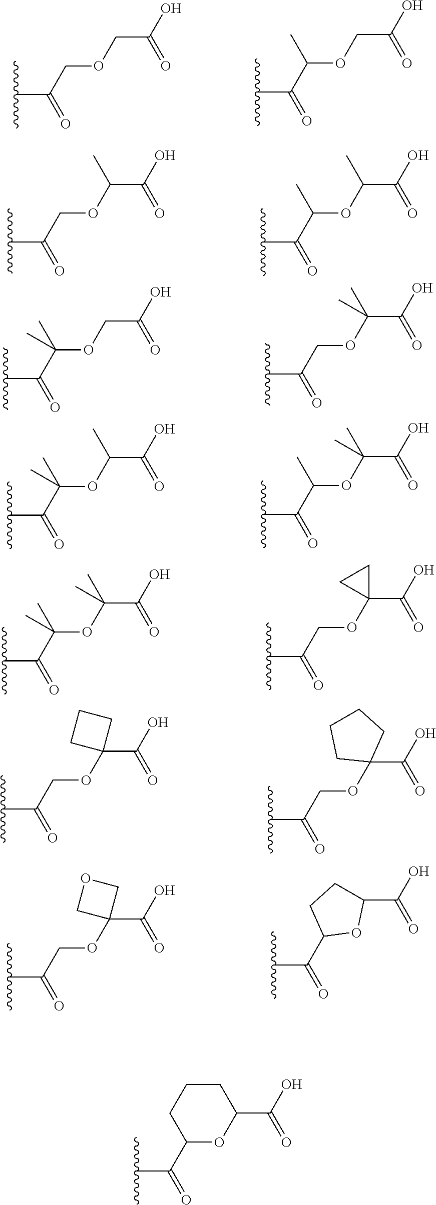

7. The compound of claim 1, wherein the X moiety is selected from:

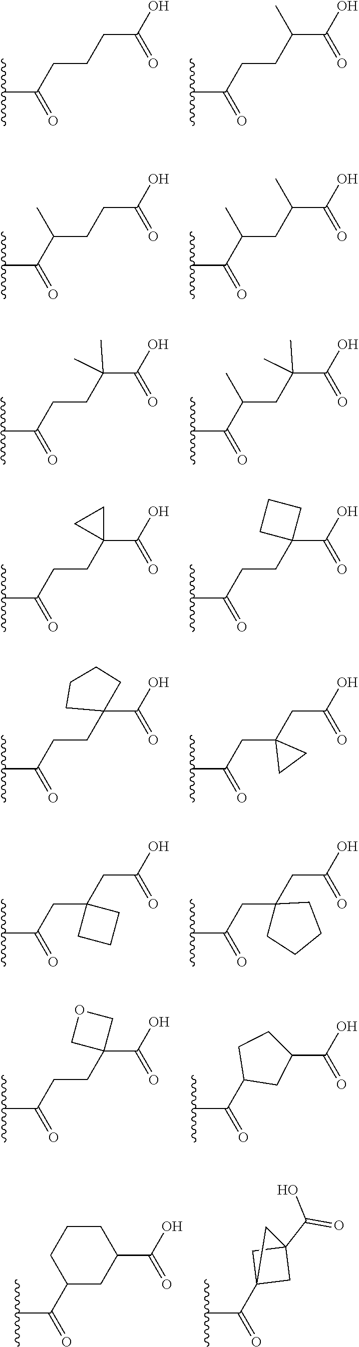

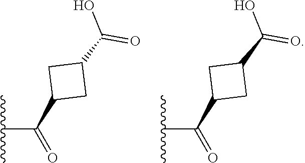

8. The compound of claim 1, wherein the compound is selected from any one of compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60:

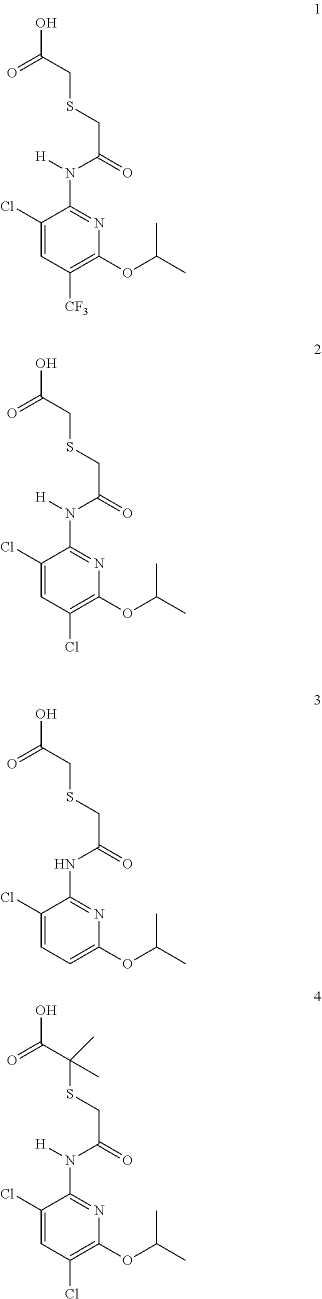 [SMILES: CC(C)Oc1nc(NC(=O)CSCC(=O)O)c(Cl)cc1C(F)(F)F ; CC(C)Oc1nc(NC(=O)CSCC(=O)O)c(Cl)cc1Cl ; CC(C)Oc1ccc(Cl)c(NC(=O)CSCC(=O)O)n1 ; CC(C)Oc1nc(NC(=O)CSC(C)(C)C(=O)O)c(Cl)cc1Cl]

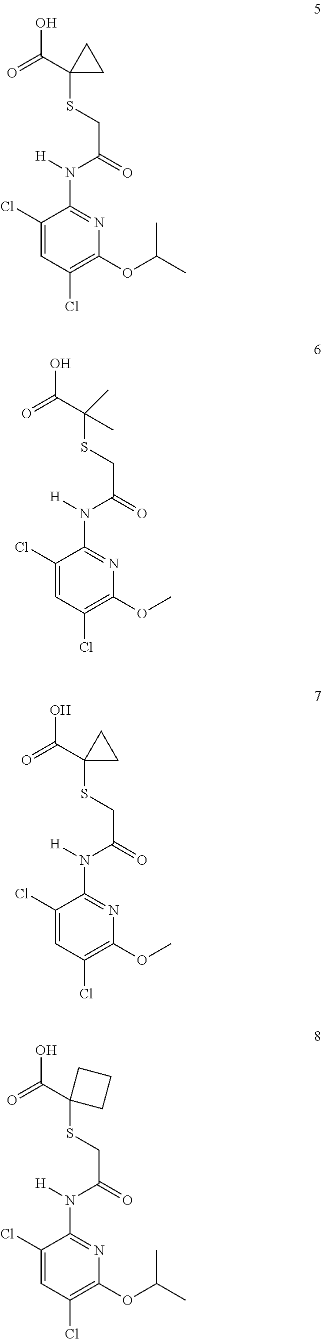 [SMILES: CC(C)Oc1nc(NC(=O)CSC2(C(=O)O)CC2)c(Cl)cc1Cl ; COc1nc(NC(=O)CSC(C)(C)C(=O)O)c(Cl)cc1Cl ; COc1nc(NC(=O)CSC2(C(=O)O)CC2)c(Cl)cc1Cl ; CC(C)Oc1nc(NC(=O)CSC2(C(=O)O)CCC2)c(Cl)cc1Cl]

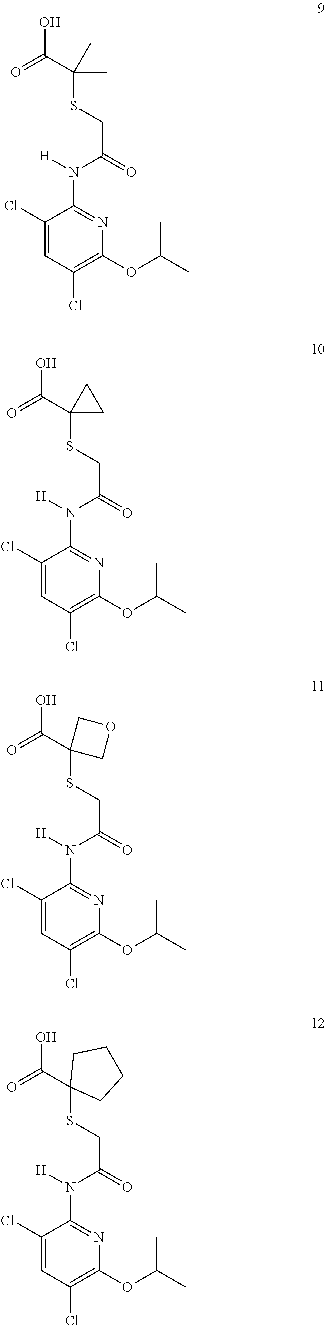 [SMILES: CC(C)Oc1nc(NC(=O)CSC(C)(C)C(=O)O)c(Cl)cc1Cl ; CC(C)Oc1nc(NC(=O)CSC2(C(=O)O)CC2)c(Cl)cc1Cl ; CC(C)Oc1nc(NC(=O)CSC2(C(=O)O)COC2)c(Cl)cc1Cl ; CC(C)Oc1nc(NC(=O)CSC2(C(=O)O)CCCC2)c(Cl)cc1Cl]

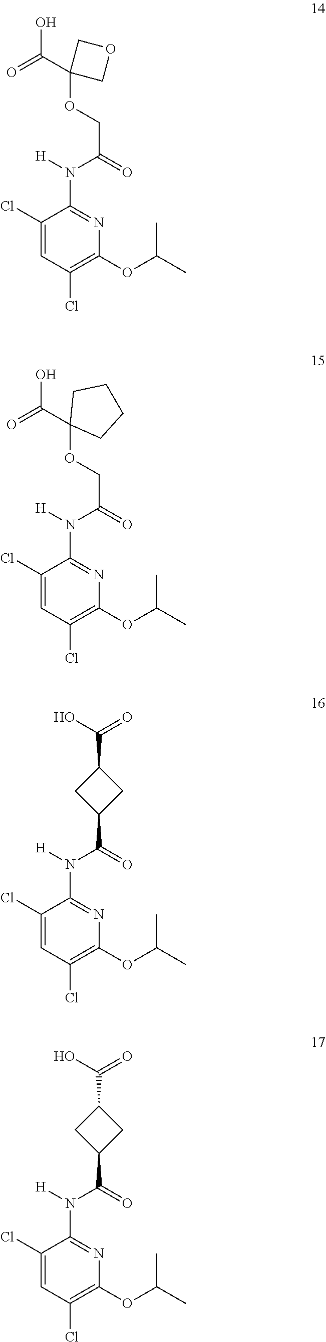 [SMILES: CC(C)Oc1nc(NC(=O)COC2(C(=O)O)COC2)c(Cl)cc1Cl ; CC(C)Oc1nc(NC(=O)COC2(C(=O)O)CCCC2)c(Cl)cc1Cl ; CC(C)Oc1nc(NC(=O)[C@H]2C[C@@H](C(=O)O)C2)c(Cl)cc1Cl ; CC(C)Oc1nc(NC(=O)[C@H]2C[C@H](C(=O)O)C2)c(Cl)cc1Cl]

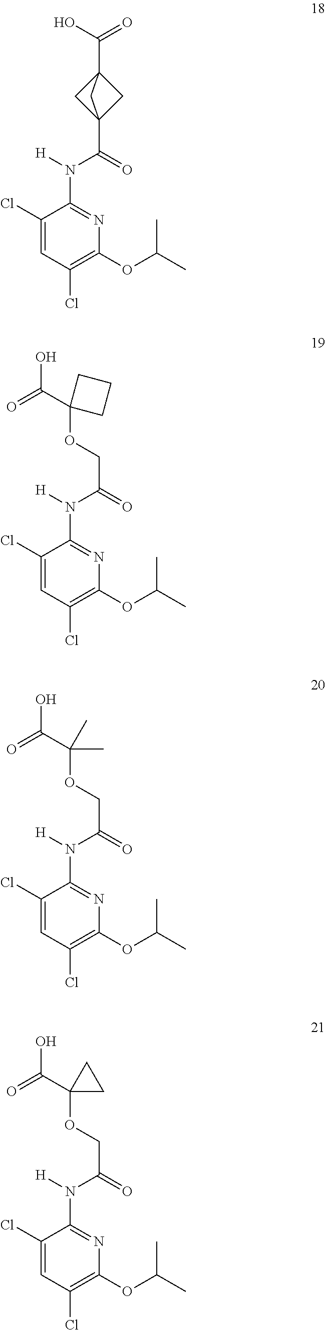 [SMILES: CC(C)Oc1nc(NC(=O)C23CC(C(=O)O)(C2)C3)c(Cl)cc1Cl ; CC(C)Oc1nc(NC(=O)COC2(C(=O)O)CCC2)c(Cl)cc1Cl ; CC(C)Oc1nc(NC(=O)COC(C)(C)C(=O)O)c(Cl)cc1Cl ; CC(C)Oc1nc(NC(=O)COC2(C(=O)O)CC2)c(Cl)cc1Cl]

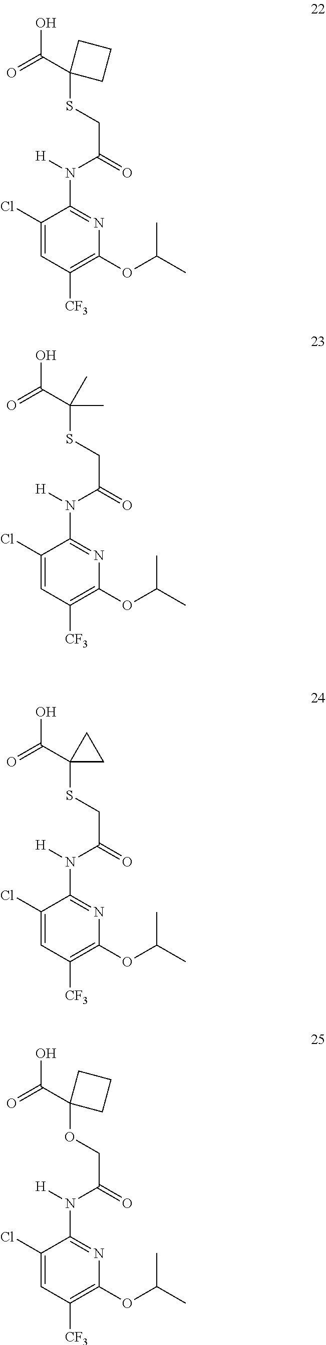 [SMILES: CC(C)Oc1nc(NC(=O)CSC2(C(=O)O)CCC2)c(Cl)cc1C(F)(F)F ; CC(C)Oc1nc(NC(=O)CSC(C)(C)C(=O)O)c(Cl)cc1C(F)(F)F ; CC(C)Oc1nc(NC(=O)CSC2(C(=O)O)CC2)c(Cl)cc1C(F)(F)F ; CC(C)Oc1nc(NC(=O)COC2(C(=O)O)CCC2)c(Cl)cc1C(F)(F)F]

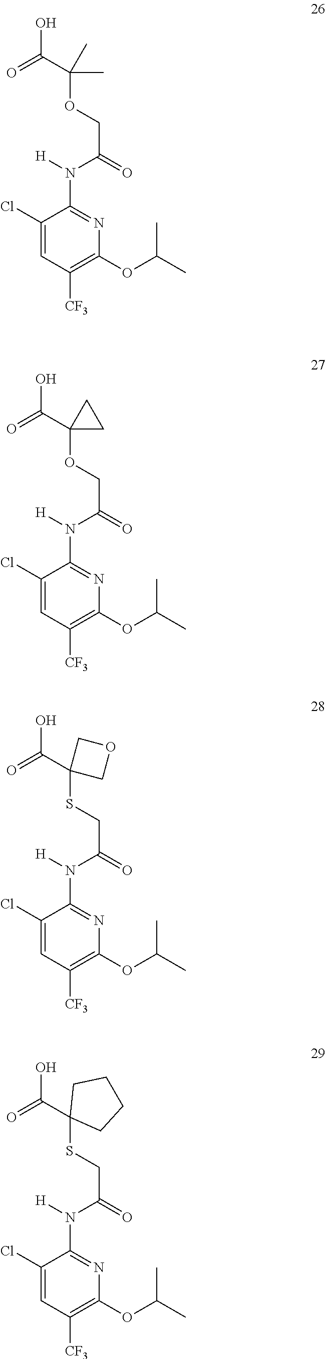 [SMILES: CC(C)Oc1nc(NC(=O)COC(C)(C)C(=O)O)c(Cl)cc1C(F)(F)F ; CC(C)Oc1nc(NC(=O)COC2(C(=O)O)CC2)c(Cl)cc1C(F)(F)F ; CC(C)Oc1nc(NC(=O)CSC2(C(=O)O)COC2)c(Cl)cc1C(F)(F)F ; CC(C)Oc1nc(NC(=O)CSC2(C(=O)O)CCCC2)c(Cl)cc1C(F)(F)F]

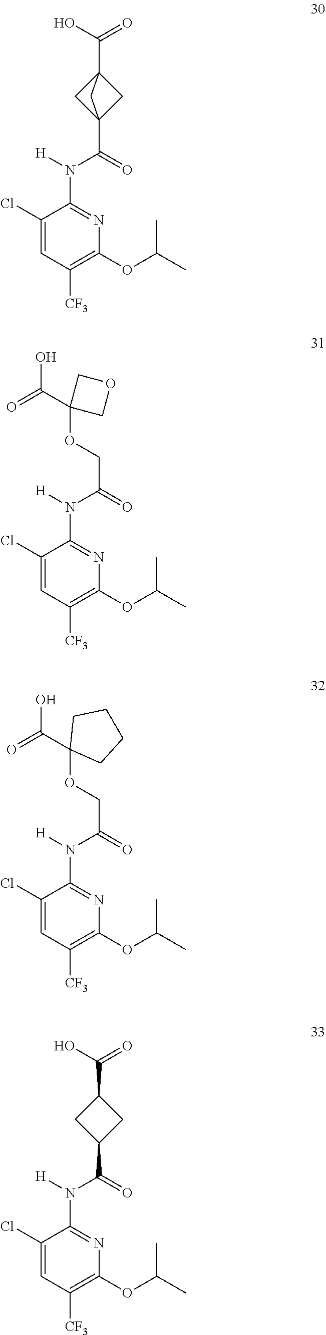 [SMILES: CC(C)Oc1nc(NC(=O)C23CC(C(=O)O)(C2)C3)c(Cl)cc1C(F)(F)F ; CC(C)Oc1nc(NC(=O)COC2(C(=O)O)COC2)c(Cl)cc1C(F)(F)F ; CC(C)Oc1nc(NC(=O)COC2(C(=O)O)CCCC2)c(Cl)cc1C(F)(F)F ; CC(C)Oc1nc(NC(=O)[C@H]2C[C@@H](C(=O)O)C2)c(Cl)cc1C(F)(F)F]

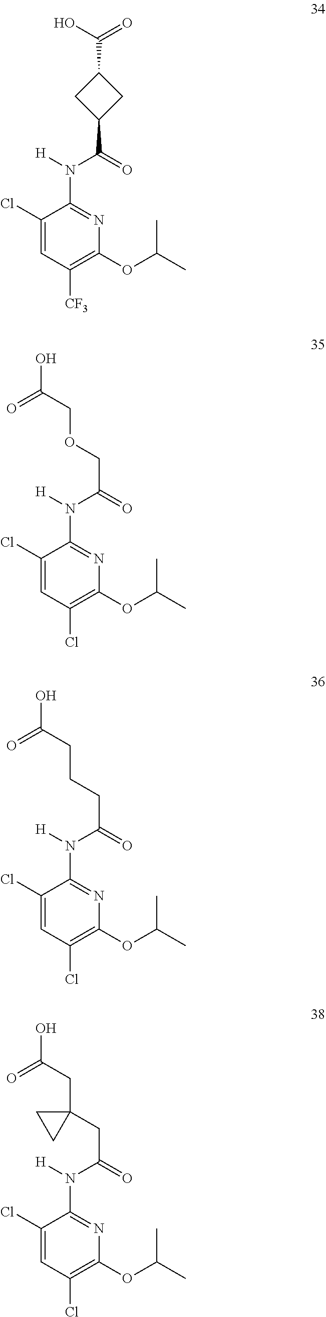 [SMILES: CC(C)Oc1nc(NC(=O)[C@H]2C[C@H](C(=O)O)C2)c(Cl)cc1C(F)(F)F ; CC(C)Oc1nc(NC(=O)COCC(=O)O)c(Cl)cc1Cl ; CC(C)Oc1nc(NC(=O)CCCC(=O)O)c(Cl)cc1Cl ; CC(C)Oc1nc(NC(=O)CC2(CC(=O)O)CC2)c(Cl)cc1Cl]

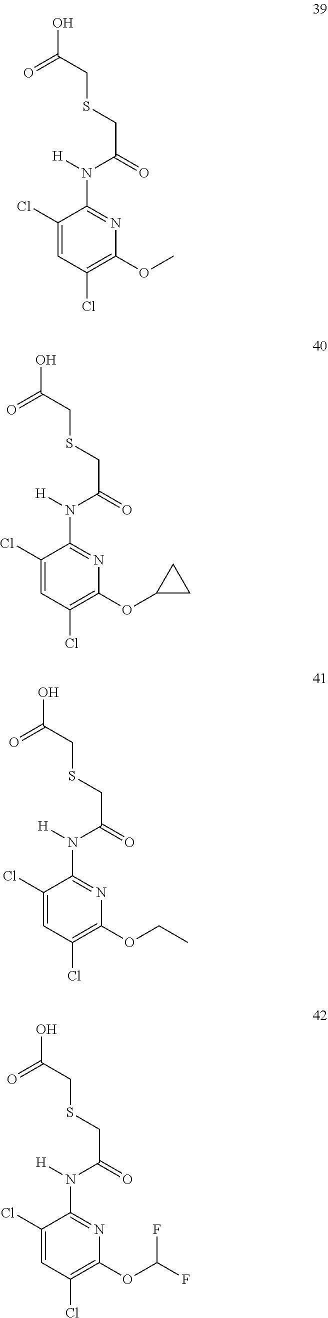 [SMILES: COc1nc(NC(=O)CSCC(=O)O)c(Cl)cc1Cl ; O=C(O)CSCC(=O)Nc1nc(OC2CC2)c(Cl)cc1Cl ; CCOc1nc(NC(=O)CSCC(=O)O)c(Cl)cc1Cl ; O=C(O)CSCC(=O)Nc1nc(OC(F)F)c(Cl)cc1Cl]

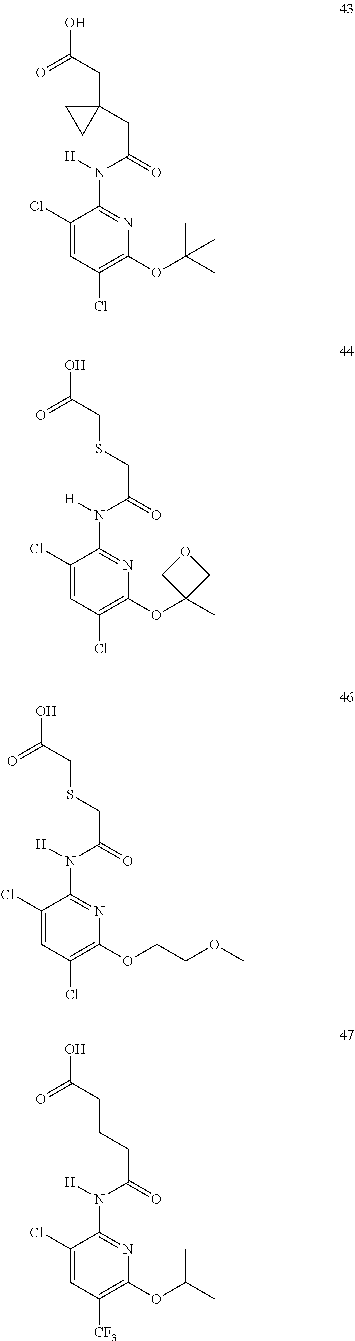 [SMILES: CC(C)(C)Oc1nc(NC(=O)CC2(CC(=O)O)CC2)c(Cl)cc1Cl ; CC1(Oc2nc(NC(=O)CSCC(=O)O)c(Cl)cc2Cl)COC1 ; COCCOc1nc(NC(=O)CSCC(=O)O)c(Cl)cc1Cl ; CC(C)Oc1nc(NC(=O)CCCC(=O)O)c(Cl)cc1C(F)(F)F]

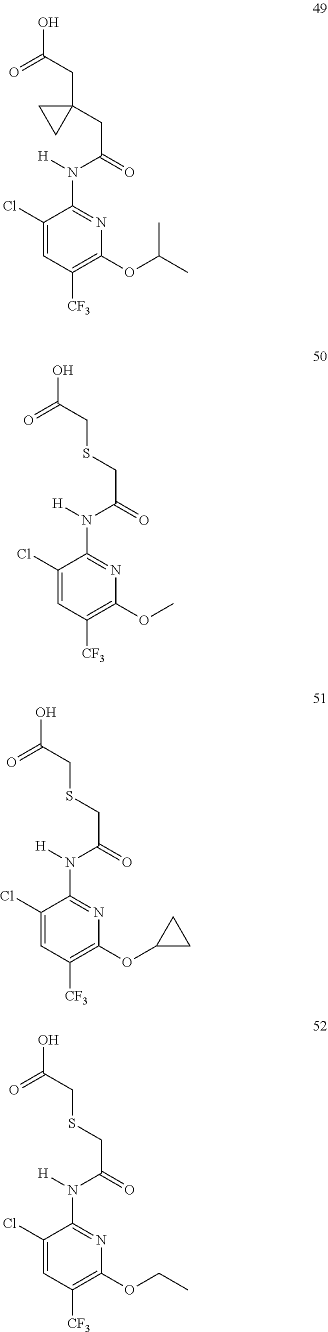 [SMILES: CC(C)Oc1nc(NC(=O)CC2(CC(=O)O)CC2)c(Cl)cc1C(F)(F)F ; COc1nc(NC(=O)CSCC(=O)O)c(Cl)cc1C(F)(F)F ; O=C(O)CSCC(=O)Nc1nc(OC2CC2)c(C(F)(F)F)cc1Cl ; CCOc1nc(NC(=O)CSCC(=O)O)c(Cl)cc1C(F)(F)F]

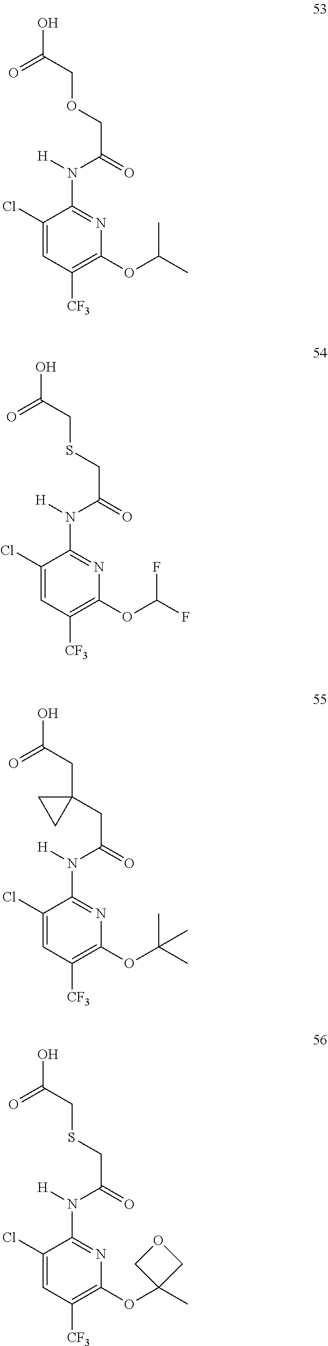 [SMILES: CC(C)Oc1nc(NC(=O)COCC(=O)O)c(Cl)cc1C(F)(F)F ; O=C(O)CSCC(=O)Nc1nc(OC(F)F)c(C(F)(F)F)cc1Cl ; CC(C)(C)Oc1nc(NC(=O)CC2(CC(=O)O)CC2)c(Cl)cc1C(F)(F)F ; CC1(Oc2nc(NC(=O)CSCC(=O)O)c(Cl)cc2C(F)(F)F)COC1]

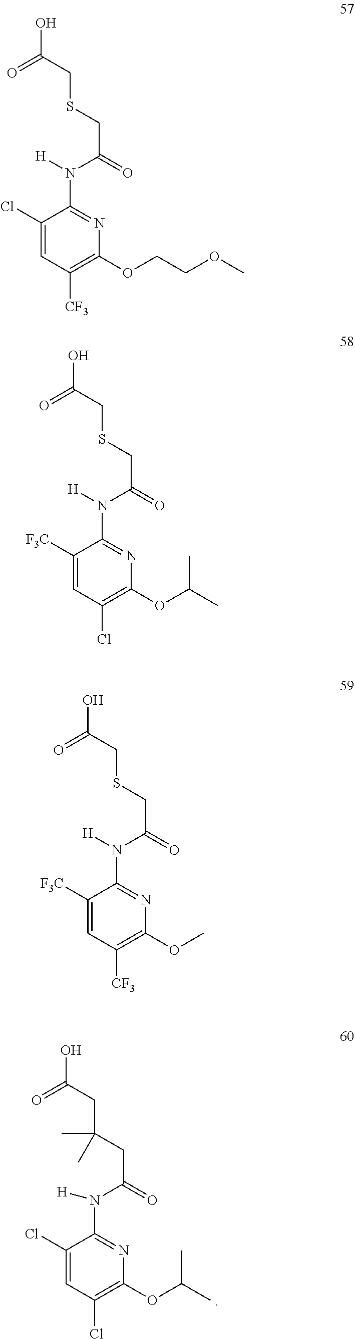 [SMILES: COCCOc1nc(NC(=O)CSCC(=O)O)c(Cl)cc1C(F)(F)F ; CC(C)Oc1nc(NC(=O)CSCC(=O)O)c(C(F)(F)F)cc1Cl ; COc1nc(NC(=O)CSCC(=O)O)c(C(F)(F)F)cc1C(F)(F)F ; CC(C)Oc1nc(NC(=O)CC(C)(C)CC(=O)O)c(Cl)cc1Cl]

9. A pharmaceutical composition, comprising a compound of claim 1 and one or more adjunct ingredients.

10. A compound of structural formula II or a pharmaceutically acceptable salt thereof, 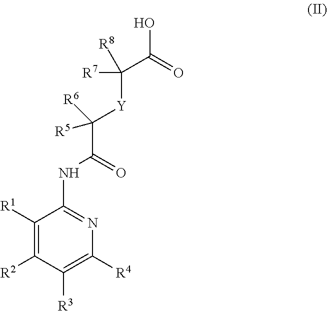
wherein,
Y is a heteroatom selected from —S—, —O—, —SO—, and —SO₂—, or is —CR⁹R¹⁰—, wherein R⁹ and R¹⁰ are each independently selected from hydrogen, C₁-C₄ linear or branched alkyl, phenyl, and benzyl, or R⁹ and R¹⁰ together form a cyclopropyl, cyclobutyl, or cyclopentyl ring;
R⁵, R⁶, R⁷, and R⁸ is each independently selected from hydrogen, C₁-C₄ linear or branched alkyl, phenyl, and benzyl, and/or
R⁵ and R⁶ together or R⁷ and R⁸ together form a cyclopropyl ring, a cyclobutyl ring, a cyclopentyl ring, or an oxetane ring,
R⁶ and R⁷ together form a 4-, 5-, or 6-membered carbocyclic or heterocyclic ring with Y as a member of the ring, or
R⁵ and R⁶ together and R⁷ and R⁸ together form a bicyclo[1.1.1] ring.

11. The compound of claim 10, wherein R¹, R², R³, and R⁴ is each independently selected from hydrogen, chlorine, fluorine, tri-fluoromethyl, methoxy, difluoro-methoxy, trifluoro-methoxy, ethoxy, propoxy, isopropyloxy, cyclopropyloxy, propargyloxy, butoxy, iso-butoxy, sec-butoxy, tert-butoxy, 2-methoxyethoxy, 2-ethoxy-2-oxoethoxy, (3-methyloxetan-3-yl)oxy, or benzyloxy.

12. The compound of claim 10, wherein:
R¹ is selected from chlorine, fluorine, methyl, and tri-fluoromethyl;
R² is hydrogen;
R³ is selected from hydrogen, chlorine, fluorine, methyl, and tri-fluoromethyl; and
R⁴ is each independently selected from methoxy, difluoro-methoxy, trifluoro-methoxy, ethoxy, propoxy, isopropoxy, cyclopropoxy, propargyloxy, butoxy, iso-butoxy, sec-butoxy, tert-butoxy, 2-methoxyethoxy, 2-ethoxy-2-oxoethoxy, (3-methyloxetan-3-yl)oxy, or benzyloxy.

13. The compound of claim 10, wherein the compound of structural formula II has a structural formula selected from IIa, IIb, IIc, IId, IIe, IIf, IIg, IIh, IIi, IIj, IIk, IIl, IIm, IIn, IIo, IIp, IIq, IIr, IIs, IIt, IIu, IIv, IIw, IIx, IIy, IIz, IIaa, IIbb, IIcc, and IIdd:

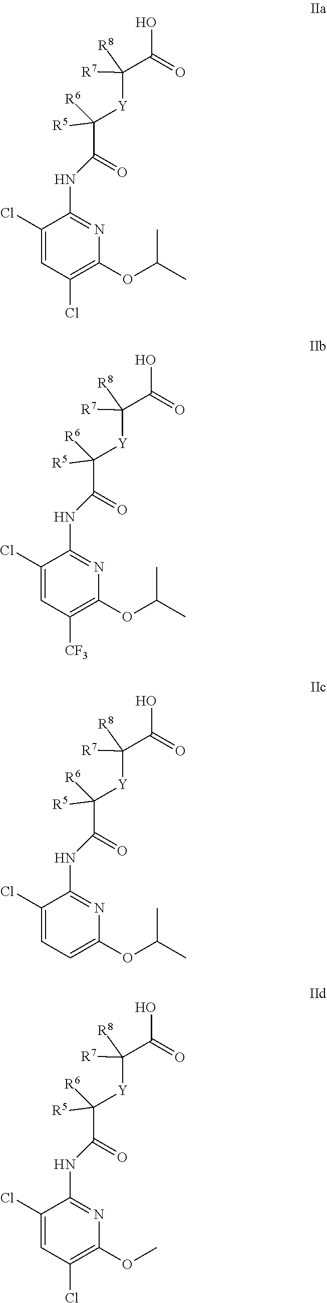

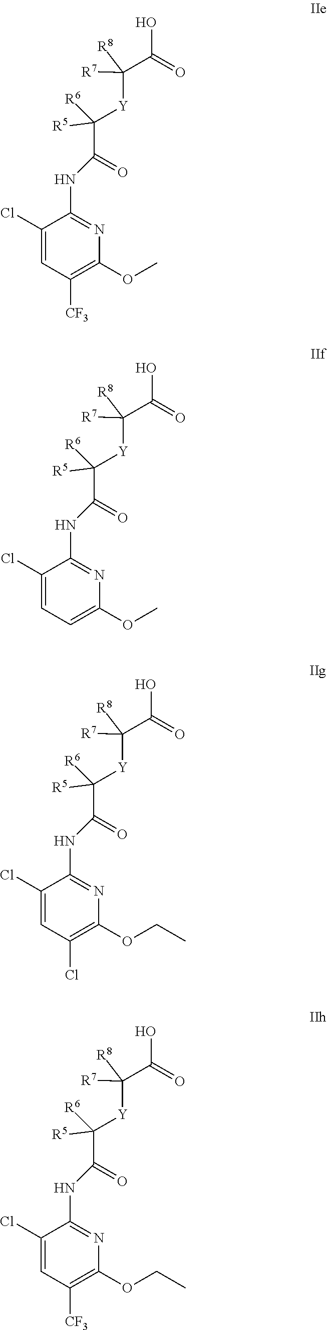

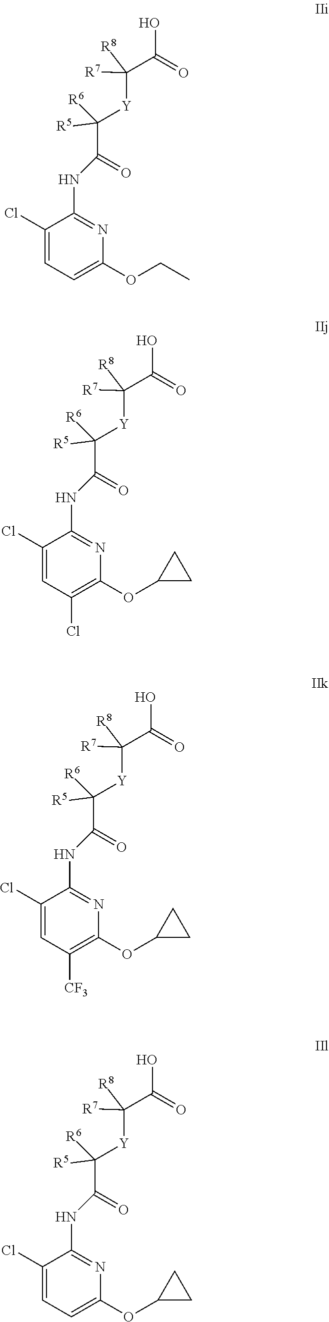

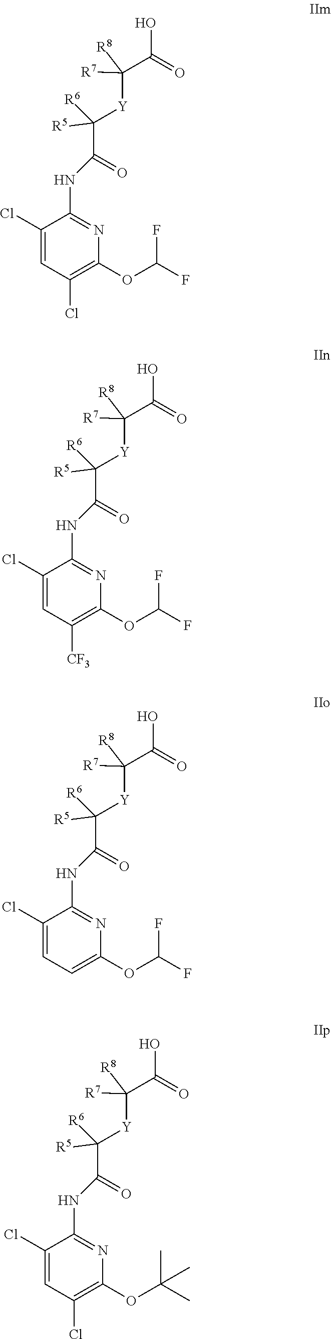

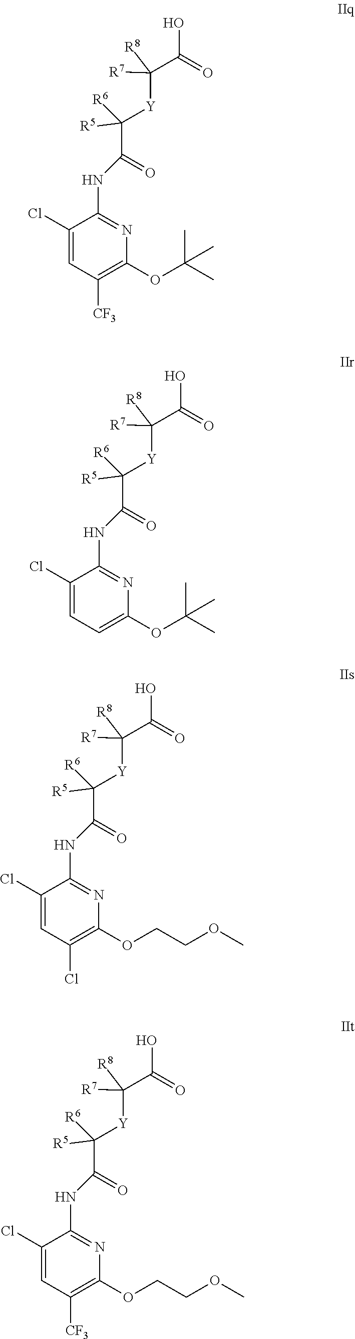

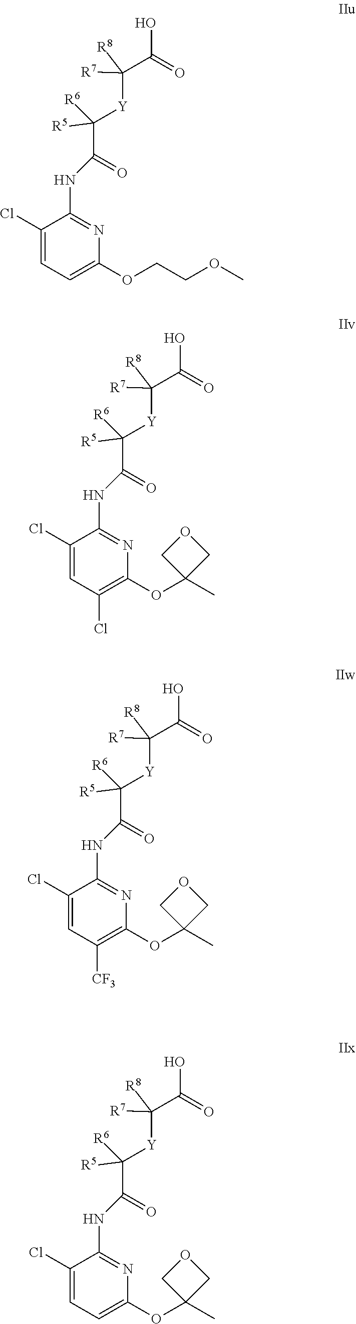

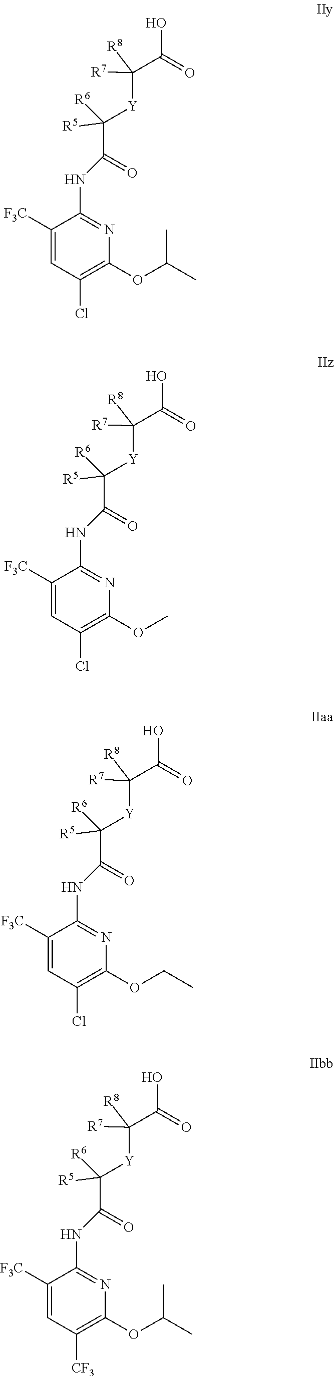

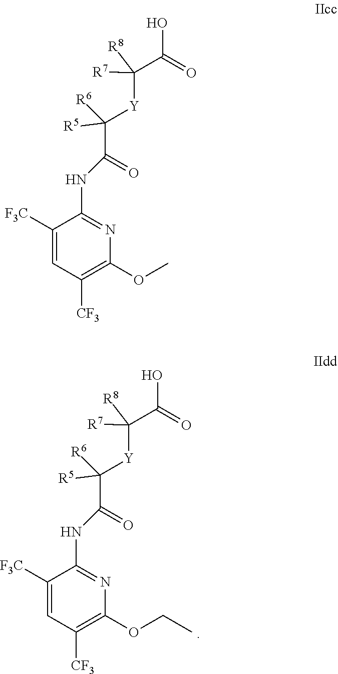

14. The compound of claim 10, wherein the compound is selected from any one of compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60:

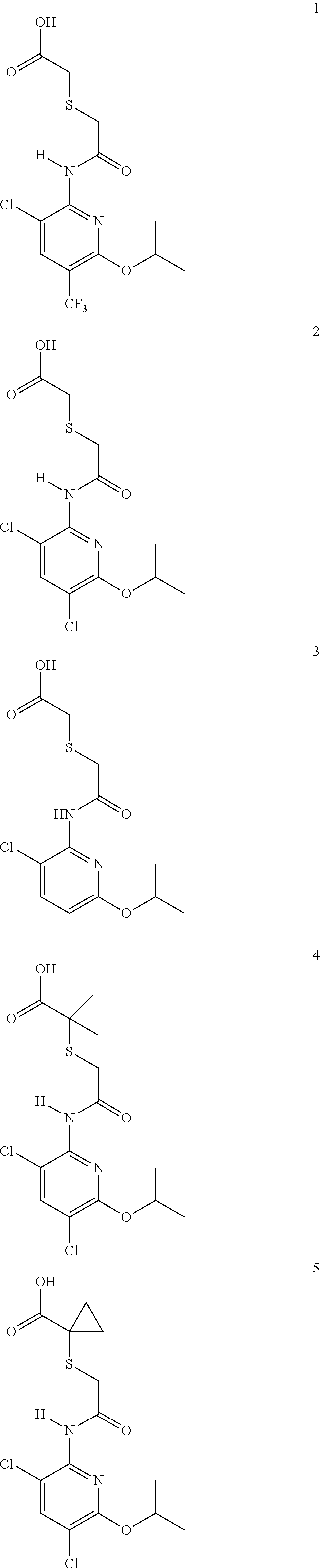

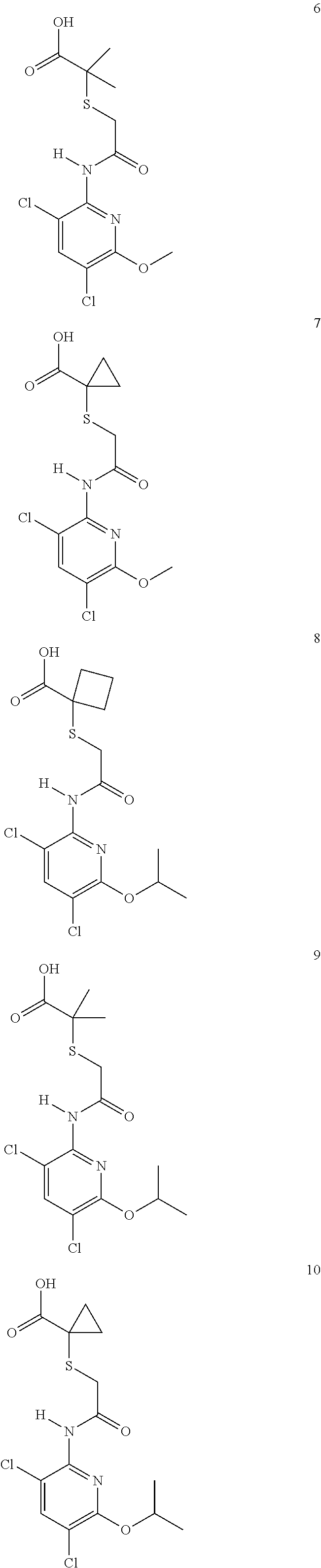

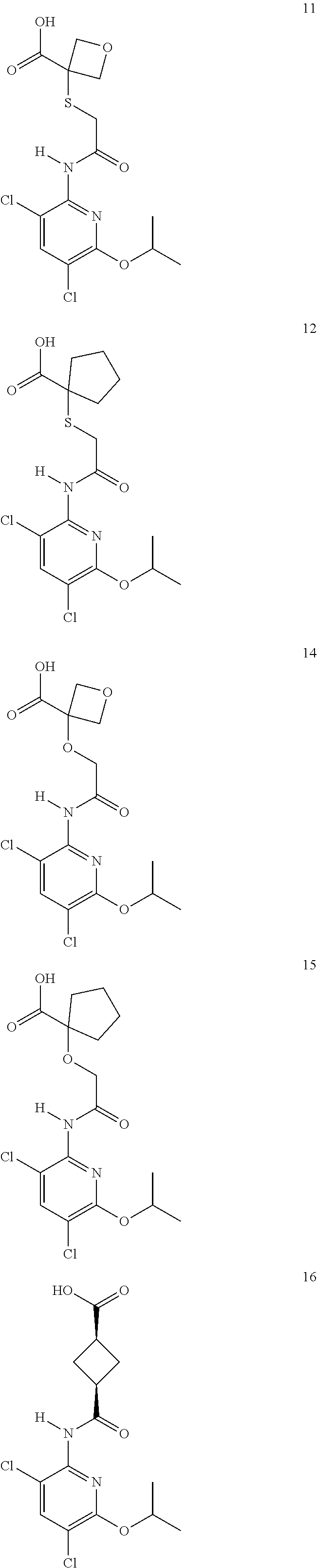

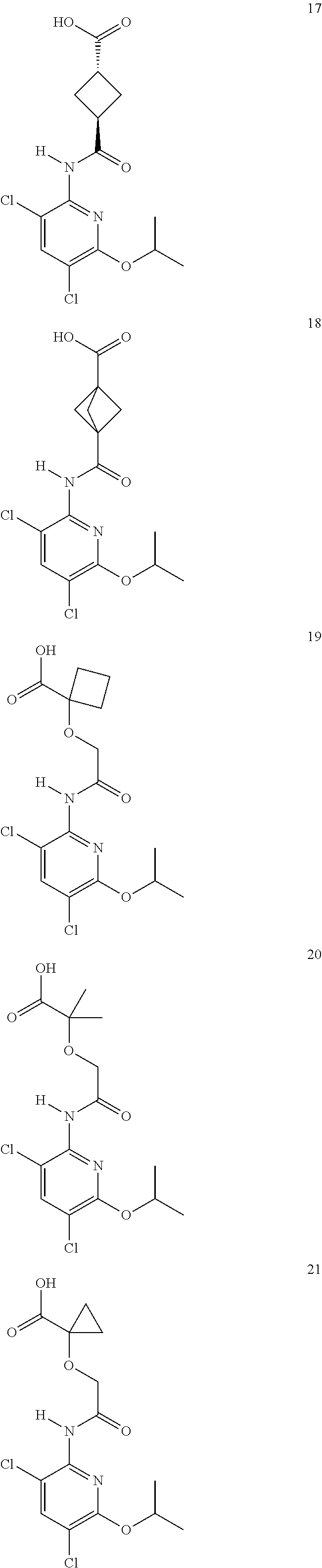

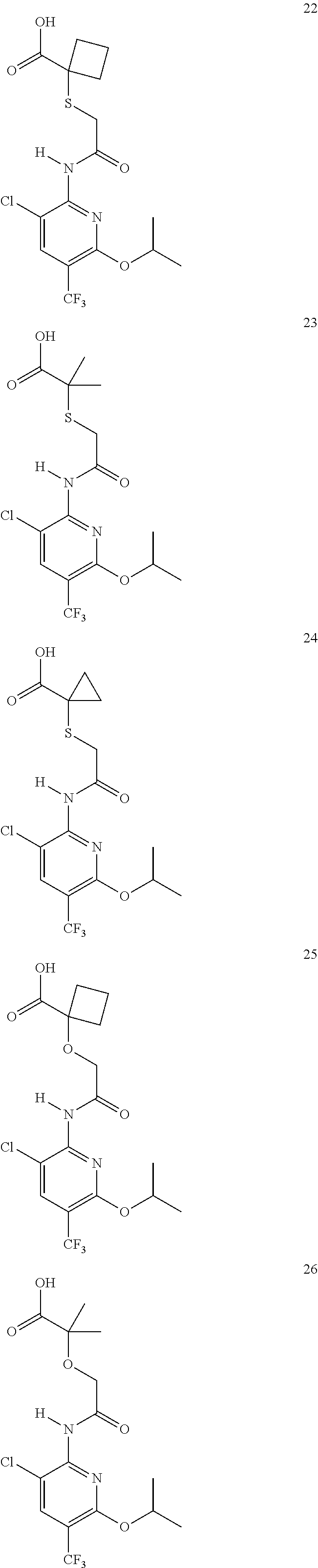 [SMILES: CC(C)Oc1nc(NC(=O)CSC2(C(=O)O)CCC2)c(Cl)cc1C(F)(F)F ; CC(C)Oc1nc(NC(=O)CSC(C)(C)C(=O)O)c(Cl)cc1C(F)(F)F ; CC(C)Oc1nc(NC(=O)CSC2(C(=O)O)CC2)c(Cl)cc1C(F)(F)F ; CC(C)Oc1nc(NC(=O)COC2(C(=O)O)CCC2)c(Cl)cc1C(F)(F)F ; CC(C)Oc1nc(NC(=O)COC(C)(C)C(=O)O)c(Cl)cc1C(F)(F)F]

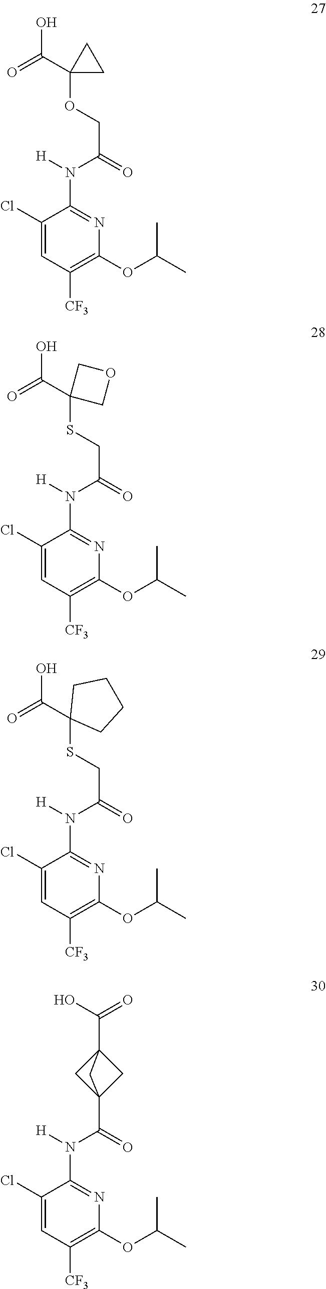 [SMILES: CC(C)Oc1nc(NC(=O)COC2(C(=O)O)CC2)c(Cl)cc1C(F)(F)F ; CC(C)Oc1nc(NC(=O)CSC2(C(=O)O)COC2)c(Cl)cc1C(F)(F)F ; CC(C)Oc1nc(NC(=O)CSC2(C(=O)O)CCCC2)c(Cl)cc1C(F)(F)F ; CC(C)Oc1nc(NC(=O)C23CC(C(=O)O)(C2)C3)c(Cl)cc1C(F)(F)F]

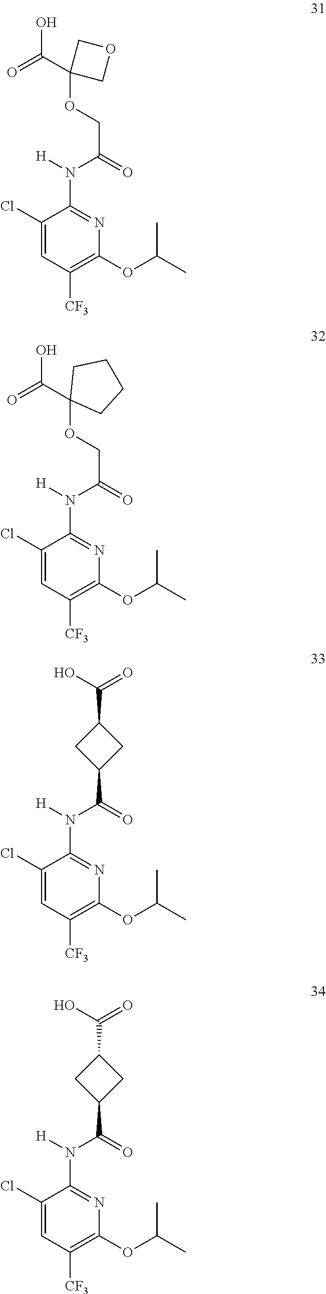

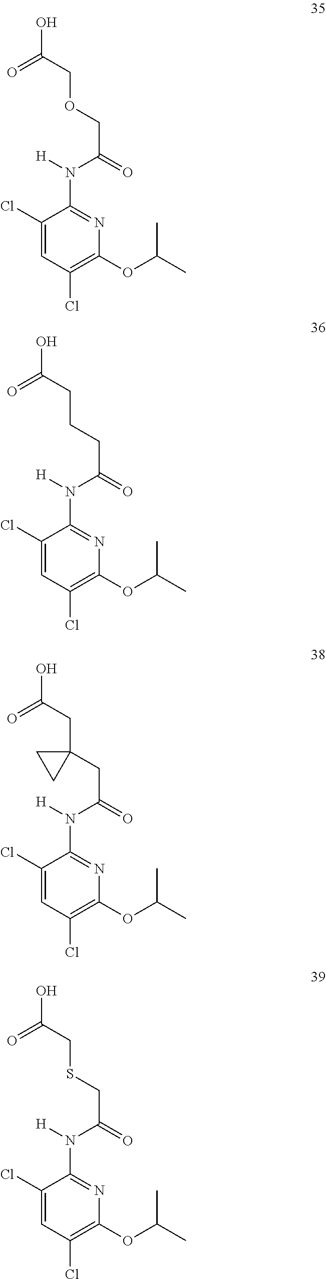

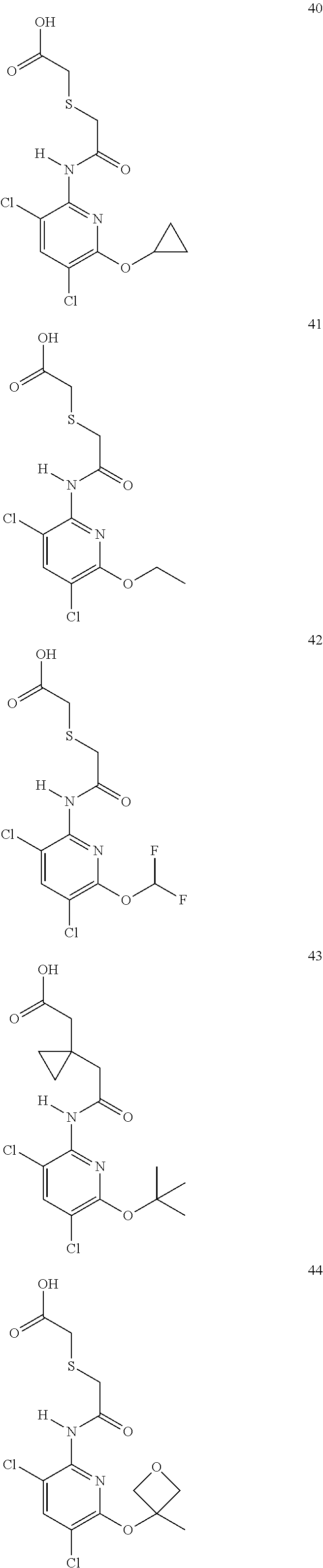

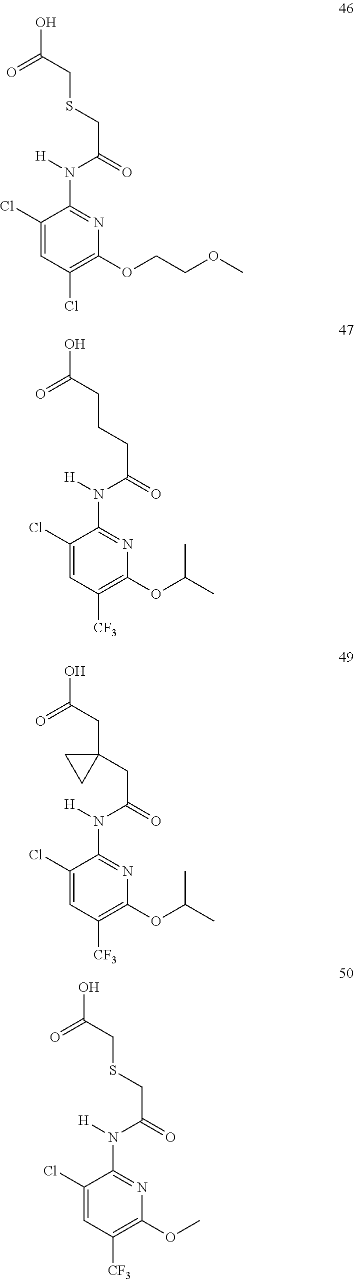

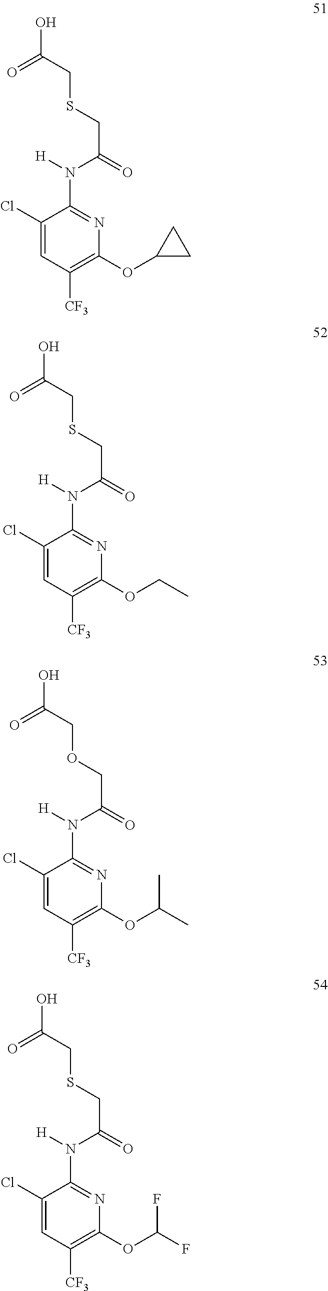 [SMILES: O=C(O)CSCC(=O)Nc1nc(OC2CC2)c(C(F)(F)F)cc1Cl ; CCOc1nc(NC(=O)CSCC(=O)O)c(Cl)cc1C(F)(F)F ; CC(C)Oc1nc(NC(=O)COCC(=O)O)c(Cl)cc1C(F)(F)F ; O=C(O)CSCC(=O)Nc1nc(OC(F)F)c(C(F)(F)F)cc1Cl]

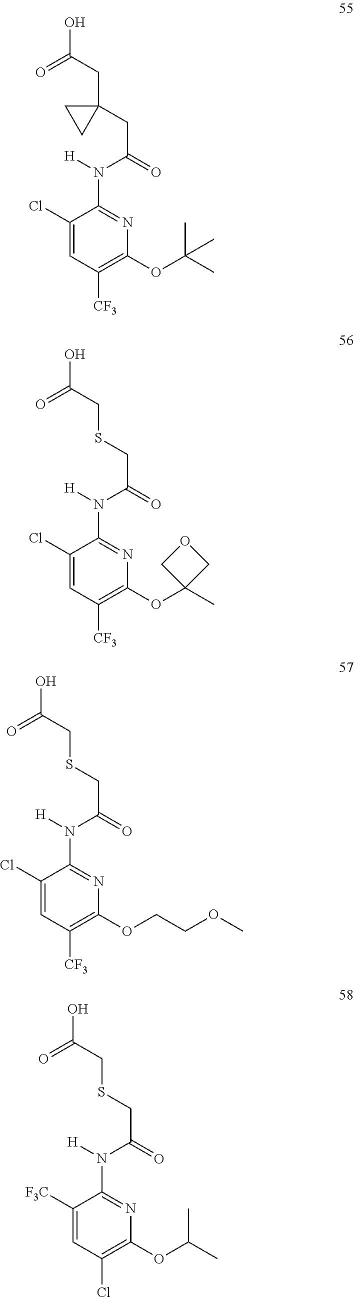

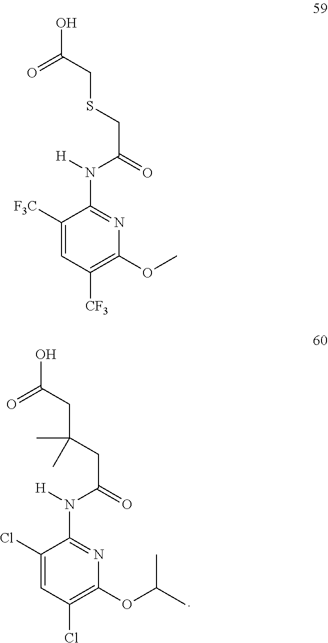 [SMILES: COc1nc(NC(=O)CSCC(=O)O)c(C(F)(F)F)cc1C(F)(F)F ; CC(C)Oc1nc(NC(=O)CC(C)(C)CC(=O)O)c(Cl)cc1Cl]

15. A pharmaceutical composition, comprising a compound of claim 10 and one or more adjunct ingredients.

16. A method for treating a subject having a disease or condition affected by FABP3/4/5/7, comprising administering to a subject in need thereof a therapeutically effective amount of a compound of claim 1.

17. The method of claim 16, wherein disease or condition affected by FABP3/4/5/7 is selected from atherosclerosis, coronary atherosclerosis, arterial fibrosis, pulmonary hypertension, heart failure, obesity, Type-2 diabetes, Type-I-diabetes, gestational diabetes, polycystic ovary syndrome, endometriosis, conditions affected by lipid metabolism and free fatty acid serum levels, metabolic disorders, fatty liver disease, kidney fibrosis, systemic inflammation, acute inflammation, allergic inflammation, airway inflammation, viral infection, vitiligo, psoriasis, atopic dermatitis, allergic contact dermatitis, mycosis fungoides, alopecia areata, cicatricial alopecia, graft versus host disease (GvHD), contact dermatitis, chronic eczema, dermatitis herpetiformis, cutaneous lupus, scleroderma, dermatomyositis, vasculitis, pemphigus, epidermolysis bullosa, linear IgA, blistering disease, neurological conditions and diseases, such as pain, multiple sclerosis (MS), Parkinson's disease, experimental autoimmune encephalomyelitis (EAE), asthma, type-I-diabetes, autoimmune lung disease, autoimmune hepatitis, rheumatoid arthritis (RA), spondyloarthropathy, vesicular stomatitis virus infection, multiple sclerosis (MS), lupus nephritis, Crohn's disease, ulcerative colitis, food allergy, ischemic stroke, breast cancer, prostate cancer, ovarian cancer, skin cancer, gastric cancer, glioma, cholangiocarcinoma, bladder cancer, multiple myeloma, colorectal cancer, hepatocellular carcinoma, cervical cancer, oral squamous cell carcinoma, and/or non-small cell lung cancer (NSCLC).

18. A process for preparing a compound of structural formula II 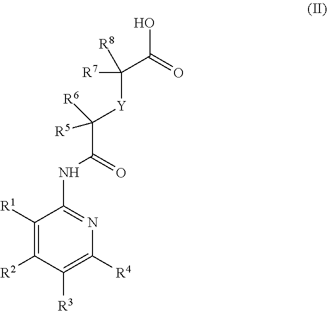
wherein,
Y is a heteroatom selected from —S—, —O—, —SO—, —SO₂—, or is —CR⁹R¹⁰—, wherein R⁹ and R¹⁰ are each independently selected from hydrogen, C₁-C₄ linear or branched alkyl, phenyl, and benzyl, or R⁹ and R¹⁰ together form a cyclopropyl, cyclobutyl, or cyclopentyl ring;
R⁵, R⁶, R⁷, and R⁸ is each independently selected from hydrogen, C₁-C₄ linear or branched alkyl, phenyl, and benzyl, and/or
R⁵ and R⁶ together or R⁷ and R⁸ together form a cyclopropyl ring, a cyclobutyl ring, a cyclopentyl ring, or an oxetane ring,
R⁶ and R⁷ together form a 4-, 5-, or 6-membered carbocyclic or heterocyclic ring with Y as a member of the ring, or
R⁵ and R⁶ together and R⁷ and R⁸ together form a bicyclo[1.1.1] ring.
the method comprising:
(a) combining in a solvent a substituted anhydride compound of formula III: 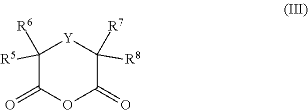
wherein Y, R⁵, R⁶, R⁷ and R⁸ are as defined above;
with a substituted pyridinyl compound of formula IV: 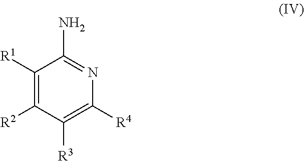
wherein, R¹, R², R³, and R⁴ are as defined above; and
(b) removing the solvent to obtain a compound having the structural formula II.
